(19) United States
(12) Patent Application Publication
(10) Pub. No.: US 2012/0283275 A1
(43) Pub. Date: Nov. 8, 2012

(54) METHODS AND DOSAGE FORMS FOR REDUCING SIDE EFFECTS OF BENZISOZAZOLE DERIVATIVES

(76) Inventors: Gayatri Sathyan, San Jose, CA (US); Suneel Gupta, Sunnyvale, CA (US); Noymi V. Yam, Sunnyvale, CA (US); Iran Reyes, San Jose, CA (US); Nipun Davar, Fremont, CA (US); Atul D. Ayer, Palo Alto, CA (US); Julie Lee, Sunnyvale, CA (US); Sonya Seroff, San Jose, CA (US)

(21) Appl. No.: 13/467,761

(22) Filed: May 9, 2012

Related U.S. Application Data
(63) Continuation of application No. 13/012,490, filed on Jan. 24, 2011, now abandoned.
Application No. 13/012,490 is a continuation of application No. 11/051,060, filed on Feb. 4, 2005, now abandoned.
Application No. 11/051,060 is a continuation-in-part of application No. 10/629,211, filed on Jul. 28, 2003, now abandoned.
(60) Provisional application No. 60/399,590, filed on Jul. 29, 2002.
(60) Provisional application No. 60/406,005, filed on Aug. 26, 2002.

(51) Int. Cl. A61K 31/519; A61P 25/18

(52) U.S. Cl. 514/259.41

(57) ABSTRACT

Disclosed are dosage forms and methods comprising benzisoxazole derivatives. More particularly, disclosed are dosage forms, methods, and new uses of benzisoxazole derivatives that substantially reduce or substantially eliminate certain side effects of the benzisoxazole derivatives when dosed to a patient.

### CROSS REFERENCE TO RELATED INVENTIONS

This application is a Continuation of U.S. Ser. No. 13/012,490 filed Jan. 24, 2011, which is a Continuation of U.S. Ser. No. 11/051,165, filed Feb. 4, 2005, now abandoned; which is a Continuation-in-Part of U.S. Ser. No. 10/629,211, filed Jul. 28, 2003, now abandoned; which claims benefit of U.S. provisional patent applications 60/399,590, filed Jul. 29, 2002, and 60/406,005 filed Aug. 26, 2002, all of which are incorporated herein by reference.

### FIELD OF THE INVENTION

The invention relates to dosage forms and methods comprising benzisoxazole derivatives. More particularly, the invention relates to dosage forms, methods, and new uses of benzisoxazole derivatives that substantially reduce or substantially eliminate certain side effects of the benzisoxazole derivatives when dosed to a patient.

### BACKGROUND

Patients presenting with psychosis can show a reduction in their symptoms after treatment with antipsychotic drugs. Traditional antipsychotic drugs were effective with some patients, but exhibited a wide range of undesirable side effects. Such side effects include parkinsonism, akathisia, acute dystonia, and tardive dyskinesia.

A class of newer antipsychotic drugs, referred to as atypical antipsychotics, have been introduced more recently. One of the benefits of atypical antipsychotics is a reduced side effect profile. However, even with the reduction in the side effect profile, undesirable side effects remain, including but not limited to orthostatic hypotension, seizures, dysphagia, and hyperprolactinemia. Examples of atypical antipsychotics include risperidone, olanzapine, and clozapine.

Risperidone is an antipsychotic agent indicated for the management of manifestations of psychotic disorders. Risperidone belongs to the chemical class, benzisoxazole derivatives. Physicians' Desk Reference, Thompson Healthcare, 56th Ed., pp. 1796-1800 (2002). Risperidone is a potent antagonist of the serotonin 5-HT2 receptor and the dopamine D2 receptor. Risperidone is also a selective antagonist at the alpha1 and alpha2 adrenergic receptors.

An immediate release tablet containing risperidone is currently marketed as Risperdal® by Janssen Pharmaceutical Products, L.P. Physicians' Desk Reference, Thompson Healthcare, 56th Ed., pp. 1796-1800 (2002). A long-lasting injectible for risperidone, Risperdal® Constar™, is also being marketed.

Paliperidone is the major active metabolite of risperidone. Risperidone is extensively metabolized in the liver to an equipotent metabolite, paliperidone, and the sum of the two compounds (active moiety) is thought to provide the clinical effect of risperidone. Paliperidone shares the characteristic D2, 5HT2A antagonism of atypical antipsychotic drugs, and a receptor-bind profile similar to risperidone. Humans can be phenotyped as (a) poor, (b) intermediate or (c) extensive risperidone metabolizers on the basis of their metabolic ratio (e.g., the ratio of urine recovery of risperidone to that of paliperidone over a period of 8 hours after oral intake of 10 mg of risperidone). The pharmacological profile of paliperidone closely resembles that of risperidone itself. Paliperidone is more fully described in U.S. Pat. No. 5,158,952. Additional compounds are disclosed in U.S. Pat. Nos. 4,804,665 and 4,458,076.

Paliperidone is practically insoluble in water. Additionally, since paliperidone has a long half-life of about one day, it is not a typical candidate for extended delivery. Risperidone has a shorter half-life but since it metabolizes to paliperidone, one can say the active moiety has a longer half-life. Side effects associated with administration of paliperidone are similar to those associated with administration of risperidone.

Physicians have noted that some of the side effects associated with risperidone administration can be alleviated by titration to the efficacious dose over several days. In effect, the patient develops a tolerance to the side effects over time. While this titration approach reduces the side effects of risperidone to the patient, it also reduced the utility of the drug, because of the effect of drug holidays.

Drug holidays arise when patients at a stabilized efficacious dose level may intentionally or unintentionally skip a dose. These drug holiday periods can be intentional, such as for medical reasons, or sometimes due to patient inadvertence, poor compliance, etc. In these patients there is a potential of significant orthostatic hypotension, or other side effects, once they resume, without titration, the stable dose that they were on prior to the drug holiday. Alternatively, patients may resume therapy with a subtherapeutic dose and titrate up again. This means that the subject would not receive the original therapeutic benefit for several more days or weeks after their drug holiday period. These problems are significant for a psychotic patient population who, for a variety of reasons, may be less compliant than other patient populations and more vulnerable to the effects of sub-therapeutic dosing.

Accordingly, while titration does address a desire to reduce side effects of risperidone, and potentially other benzisoxazole derivatives, it can cause problems in typical usage scenarios. There remains a need for effective dosing methods, dosage forms and devices that will permit the dosing of benzisoxazole derivatives in a way that does not require titration. Exemplary methodologies, dosage forms, methods of preparing such dosage forms and methods of using such dosage forms are disclosed herein.

### SUMMARY OF THE INVENTION

In an aspect, the invention relates to an oral dosage form and to methods of administering the dosage form wherein the oral dosage form comprises a dose D of a benzisoxazole derivative; and an oral sustained release dosing structure adapted to sustainably release the benzisoxazole derivative at rates that provide (a) a mean, single dose, maximum plasma concentration Cmax of the benzisoxazole derivative and pharmacologically active metabolites thereof taken together and (b) a mean, single dose, area under a plasma concentration-time curve for AUC_{inf} of the benzisoxazole derivative and pharmacologically active metabolites thereof taken together, which satisfy the relationships:

about 0.5 ng/mL/mg≦Cmax/D≦about 8 ng/mL/mg, and

about 30 ng hr/mL/mg≦AUC_{inf}/D≦about 300 ng hr/mL/mg.

In an aspect, the invention relates to an oral dosage form and to methods of administering the dosage form wherein the oral dosage form comprises a dose D of a benzisoxazole derivative; and an oral sustained release dosing structure adapted to sustainably release the benzisoxazole derivative at rates that provide (a) a mean, single dose, maximum plasma concentration Cmax of the benzisoxazole derivative and pharmacologically active metabolites thereof taken together and (b) a mean, single dose, time to maximum plasma concentration Tmax of the benzisoxazole derivative, which satisfy the relationships:

about 0.5 ng/mL/mg≦Cmax/D≦about 8 ng/mL/mg,

and

Tmax≧about 9 hours.

In an aspect, the invention relates to an oral dosage form and to methods of administering the dosage form wherein the oral dosage form comprises a dose D of a benzisoxazole derivative; and an oral sustained release dosing structure adapted to sustainably release the benzisoxazole derivative at rates that provide (a) a mean, single dose, area under a plasma concentration-time curve for AUC_{inf} of the benzisoxazole derivative and pharmacologically active metabolites thereof taken together and (b) a mean, single dose, time to maximum plasma concentration Tmax of the benzisoxazole derivative, which satisfy the relationships:

about 30 ng hr/mL/mg≦AUC_{inf}/D≦about 300 ng hr/mL/mg,

and

Tmax≧about 9 hours.

In an aspect, the invention relates to an oral dosage form and to methods of administering the dosage form wherein the oral dosage form comprises a dose D of a benzisoxazole derivative; and an oral sustained release dosing structure adapted to sustainably release the benzisoxazole derivative at rates that provide (a) steady-state maximal plasma concentration of the benzisoxazole derivative and pharmacologically active metabolites thereof taken together Cₘₐₓ₋ₛₛ and (b) steady-state minimal plasma concentration of the benzisoxazole derivative and metabolites thereof taken together Cₘᵢₙ₋ₛₛ, which satisfy the relationships:

about 0.5≧Cₘₐₓ₋ₛₛ/Cₘᵢₙ₋ₛₛ≦about 1.0.

In an aspect, the invention relates to an oral dosage form and to methods of administering the dosage form wherein the oral dosage form comprises a dose of a benzisoxazole derivative; and an oral sustained release dosing structure adapted to sustainably release the benzisoxazole derivative in vitro such that:
(a) the release rate measured at 2 hours following administration of the oral dosage form does not exceed about 8 wt % of the dose per hour,
(b) the release rate measured at 6 hours following administration of the oral dosage form ranging from between about 6 to about 12 wt % of the dose per hour,
(c) the release rate measured at 10 hours following administration of the oral dosage form ranging from between about 12 to about 16 wt % of the dose per hour, or
(d) the release rate measured at 14 hours following administration of the oral dosage form does not exceed 8 wt % of the dose per hour,
wherein the release profile is determined using a USP Type VII bath indexer in a constant temperature water bath at 37° C.

In an aspect, the invention relates to an oral dosage form and to methods of administering the dosage form wherein the oral dosage form comprises a dose of a benzisoxazole derivative; and an oral sustained release dosing structure adapted to sustainably release the benzisoxazole derivative in vitro such that:
(a) the release rate measured at 2 hours following administration of the oral dosage form does not exceed about 4 wt % of the dose per hour,
(b) the release rate measured at 6 hours following administration of the oral dosage form ranging from between about 1 to about 4 wt % of the dose per hour,
(c) the release rate measured at 10 hours following administration of the oral dosage form ranging from between about 3 to about 7 wt % of the dose per hour, or
(d) the release rate measured at 24 hours following administration of the oral dosage form does not exceed 4 wt % of the dose per hour,
   wherein the release profile is determined using a USP Type VII bath indexer in a constant temperature water bath at 37° C.

In an aspect, the invention relates to an oral dosage form and to methods of administering the dosage form wherein the oral dosage form comprises a dose D of a benzisoxazole derivative; and an oral sustained release dosing structure adapted to sustainably release the benzisoxazole derivative at rates that provide (a) a mean, single dose, areas under a plasma concentration-time curve AUC of the benzisoxazole derivative and pharmacologically active metabolites thereof taken together for 0-3 hours AUC₀₋₃, 3-6 hours AUC₃₋₆, 6-9 hours AUC₆₋₉, 9-12 hours AUC₉₋₁₂, and 0-12 hours AUC₀₋₁₂ which satisfy the relationships:

AUC₀₋₃/AUC₀₋₁₂≧0.18,

AUC₃₋₆/AUC₀₋₁₂≧0.18,

AUC₆₋₉/AUC₀₋₁₂≧0.18,

and

AUC₉₋₁₂/AUC₀₋₁₂≧0.18.

In an aspect, the invention relates to an oral dosage form and to methods of administering the dosage form wherein the oral dosage form comprises a dose D of a benzisoxazole derivative; and an oral sustained release dosing structure adapted to sustainably release the benzisoxazole derivative at rates that provide (a) a mean, single dose, areas under a plasma concentration-time curve AUC of the benzisoxazole derivative and pharmacologically active metabolites thereof taken together for 0-6 hours AUC₀₋₆, 6-12 hours AUC₆₋₁₂, and 0-12 hours AUC₀₋₁₂ which satisfy the relationships:

AUC₀₋₆/AUC₀₋₁₂≧0.36,

AUC₆₋₁₂/AUC₀₋₁₂≧0.36.

In an aspect, the invention relates to an oral dosage form and to methods of administering the dosage form wherein the oral dosage form comprises a dose D of a benzisoxazole derivative; and an oral sustained release dosing structure adapted to sustainably release the benzisoxazole derivative at rates that provide (a) a mean, single dose, areas under a plasma concentration-time curve AUC of the benzisoxazole derivative and pharmacologically active metabolites thereof taken together for 0-3 hours AUC₀₋₃, 3-6 hours AUC₃₋₆, 6-9 hours AUC₆₋₉, and 9-12 hours AUC₉₋₁₂, wherein the ratios of each of: AUC₀₋₃/AUC₃₋₆,

AUC₃₋₆/AUC₆₋₉,

and

AUC₆₋₉/AUC₉₋₁₂

are between about 0.7 to about 1.33.

In an aspect, the invention relates to an oral dosage form and to methods of administering the dosage form wherein the oral dosage form comprises a dose D of a benzisoxazole derivative; and an oral sustained release dosing structure adapted to sustainably release the benzisoxazole derivative at rates that provide (a) a mean, single dose, areas under a plasma concentration-time curve AUC of the benzisoxazole derivative and pharmacologically active metabolites thereof taken together for 0-6 hours AUC₀₋₆, 6-12 hours AUC₆₋₁₂, 12-18 hours AUC₁₂₋₁₈, 18-24 hours AUC₁₈₋₂₄, and 0-24 hours AUC₀₋₂₄ which satisfy the relationships:

AUC₀₋₆/AUC₀₋₂₄≧0.18,

AUC₆₋₁₂/AUC₀₋₂₄≧0.18,

AUC₁₂₋₁₈/AUC₀₋₂₄≧0.18,

and

AUC₁₈₋₂₄/AUC₀₋₂₄≧0.18.

In an aspect, the invention relates to an oral dosage form and to methods of administering the dosage form wherein the oral dosage form comprises a dose D of a benzisoxazole derivative; and an oral sustained release dosing structure adapted to sustainably release the benzisoxazole derivative at rates that provide (a) a mean, single dose, areas under a plasma concentration-time curve AUC of the benzisoxazole derivative and pharmacologically active metabolites thereof taken together for 0-12 hours AUC₀₋₁₂, 12-24 hours AUC₁₂₋₂₄, and 0-24 hours AUC₀₋₂₄ which satisfy the relationships:

AUC₀₋₁₂/AUC₀₋₂₄≧0.36,

AUC₁₂₋₂₄/AUC₀₋₂₄≧0.36.

In an aspect, the invention relates to an oral dosage form and to methods of administering the dosage form wherein the oral dosage form comprises a dose D of a benzisoxazole derivative; and an oral sustained release dosing structure adapted to sustainably release the benzisoxazole derivative at rates that provide (a) a mean, single dose, areas under a plasma concentration-time curve AUC of the benzisoxazole derivative and pharmacologically active metabolites thereof taken together for 0-6 hours AUC₀₋₆, 6-12 hours AUC₆₋₁₂, 12-18 hours AUC₁₂₋₁₈, and 18-24 hours AUC₁₈₋₂₄, wherein the ratios:

AUC₀₋₆/AUC₆₋₁₂

AUC₆₋₁₂/AUC₁₂₋₁₈,

and

AUC₁₂₋₁₈/AUC₁₈₋₂₄

are between about 0.7 to about 1.33.

In an aspect, the invention relates to an oral dosage form and to methods of administering the dosage form wherein the oral dosage form comprises a dose D of a benzisoxazole derivative; and an oral sustained release dosing structure adapted to sustainably release the benzisoxazole derivative at rates that provide (a) a mean, single dose, areas under a plasma concentration-time curve AUC of the benzisoxazole derivative and pharmacologically active metabolites thereof taken together for 0-3 hours AUC₀₋₃, 3-6 hours AUC₃₋₆, 6-9 hours AUC₆₋₉, 9-12 hours AUC₉₋₁₂, and 0-12 hours AUC₀₋₁₂ which satisfy the relationships:

about 0.05≧AUC₀₋₃/AUC₀₋₁₂≦about 0.15,

about 0.1≧AUC₃₋₆/AUC₀₋₁₂≦about 0.3,

about 0.15≧AUC₆₋₉/AUC₀₋₁₂≦about 0.45,

and

about 0.15≧AUC₉₋₁₂/AUC₀₋₁₂≦about 0.55.

In an aspect, the invention relates to an oral dosage form and to methods of administering the dosage form wherein the oral dosage form comprises a dose D of a benzisoxazole derivative; and an oral sustained release dosing structure adapted to sustainably release the benzisoxazole derivative at rates that provide (a) a mean, single dose, areas under a plasma concentration-time curve AUC of the benzisoxazole derivative and pharmacologically active metabolites thereof taken together for 0-6 hours AUC₀₋₆, 6-12 hours AUC₆₋₁₂, and 0-12 hours AUC₀₋₁₂ which satisfy the relationships:

about 0.1≧AUC₀₋₆/AUC₀₋₁₂≦about 0.5,

and

about 0.5≧AUC₆₋₁₂/AUC₀₋₁₂≦about 0.9.

In an aspect, the invention relates to an oral dosage form and to methods of administering the dosage form wherein the oral dosage form comprises a dose D of a benzisoxazole derivative; and an oral sustained release dosing structure adapted to sustainably release the benzisoxazole derivative at rates that provide (a) a mean, single dose, areas under a plasma concentration-time curve AUC of the benzisoxazole derivative and pharmacologically active metabolites thereof taken together for 0-6 hours AUC₀₋₆, 6-12 hours AUC₆₋₁₂, 12-18 hours AUC₁₂₋₁₈, 18-24 hours AUC₁₈₋₂₄, and 0-24 hours AUC₀₋₂₄ which satisfy the relationships:

about 0.02≧AUC₀₋₆/AUC₀₋₂₄≦about 0.2,

about 0.1≧AUC₆₋₁₂/AUC₀₋₂₄≦about 0.35,

about 0.2≧AUC₁₂₋₁₈/AUC₀₋₂₄≦about 0.4,

and

about 0.25≧AUC₁₈₋₂₄/AUC₀₋₂₄≦about 0.5.

In an aspect, the invention relates to an oral dosage form and to methods of administering the dosage form wherein the oral dosage form comprises a dose D of a benzisoxazole derivative; and an oral sustained release dosing structure adapted to sustainably release the benzisoxazole derivative at rates that provide (a) a mean, single dose, areas under a plasma concentration-time curve AUC of the benzisoxazole derivative and pharmacologically active metabolites thereof taken together for 0-12 hours AUC₀₋₁₂, 12-24 hours AUC₁₂₋₂₄, and 0-24 hours AUC₀₋₂₄ which satisfy the relationships:

about 0.10≧AUC₀₋₁₂/AUC₀₋₂₄≦about 0.55,

and

about 0.45≧AUC₁₂₋₂₄/AUC₀₋₂₄≦about 0.9.

### BRIEF DESCRIPTION OF THE DRAWINGS

FIG. 1 shows a dosage form useful in the practice of the invention.

FIG. 2 shows a dosage form useful in the practice of the invention.

FIG. 3 shows a dosage form useful in the practice of the invention.

FIG. 4 shows a dosage form useful in the practice of the invention.

FIGS. 5A-C show a dosage form useful in the practice of the invention.

FIGS. 6A-C show mean plasma concentration profiles of risperidone, paliperidone and active moiety after risperidone treatment.

FIG. 7 shows mean plasma concentration profiles of serum prolactin after risperidone treatment.

FIG. 8 shows mean plasma concentration profiles of paliperidone after paliperidone treatment.

FIG. 9 shows mean plasma concentration profiles of serum prolactin after paliperidone and placebo treatment.

FIGS. 10A-B show mean plasma concentration profiles of paliperidone and active moiety after a risperidone treatment.

FIG. 11 shows mean plasma concentration profiles of serum prolactin after risperidone, paliperidone and placebo treatment.

FIG. 12 shows mean plasma concentration profiles of paliperidone after paliperidone treatment.

FIGS. 13A-C show mean plasma concentration profiles of risperidone, paliperidone and active moiety after risperidone treatment.

FIG. 14 shows steady state active moiety profiles.

### DETAILED DESCRIPTION

### I. Introduction/Solution Summary

The inventors have unexpectedly discovered that the aforementioned problems can be addressed by providing oral dosage forms and methods that comprise a dose of a benzisoxazole derivative and dosing structures or sustainable releasing means that provide specified release profiles or, upon dosing, specified pharmacokinetic characteristics.

The present invention thus accomplishes an object of the invention of providing effective dosing methods, dosage forms and devices that will permit the dosing of benzisoxazole derivatives in a way that does not require titration. Of particular importance is the discovery that there are selected dosing structures or sustainable releasing means, and equivalents thereof, that accomplish an object of the invention. The inventive dosing structures or sustainable releasing means are distinguishable from other, non-inventive, structures or means because the inventive dosing structures or sustainable releasing means, and equivalents thereof, permit the dosing of benzisoxazole derivatives in a way that does not require titration. In contrast, other, non-inventive, dosing structures or sustainable releasing means do not permit such dosing.

The invention will now be described in more detail below.

### II. Definitions

All percentages are weight percent unless otherwise noted.

All publications cited to herein are incorporated by reference in their entirety and for all purposes as if reproduced fully herein.

The present invention is best understood by reference to the following definitions, the drawings and exemplary disclosure provided herein.

“Administering” or “administration” means providing a drug to a patient in a manner that is pharmacologically useful.

“Apparent terminal half-life” (t_{1/2}) is calculated as 0.693/k, wherein “k” means the apparent elimination rate constant, estimated by linear regression of the log-transformed plasma concentration during the terminal log-linear elimination phase.

“Area under the curve” or “AUC” is the area as measured under a plasma drug concentration curve. Often, the AUC is specified in terms of the time interval across which the plasma drug concentration curve is being integrated, for instance AUC_{start-finish}. Thus, AUC₀₋₄₈ refers to the AUC obtained from integrating the plasma concentration curve over a period of zero to 48 hours, where zero is conventionally the time of administration of the drug or dosage form comprising the drug to a patient. AUCₜ refers to area under the plasma concentration curve from hour 0 to the last detectable concentration at time t, calculated by the trapezoidal rule. AUC_{inf} refers to the AUC value extrapolated to infinity, calculated as the sum of AUCₜ and the area extrapolated to infinity, calculated by the concentration at time t (Ct) divided by k. (If the t_{1/2} value was not estimable for a subject, the mean t_{1/2} value of that treatment was used to calculate AUC_{inf}). “Mean, single dose, area under a plasma concentration-time curve AUC_{inf}” means the mean AUC_{inf} obtained over several patients or multiple administrations to the same patient on different occasions with sufficient washout in between dosings to allow drug levels to subside to pre-dose levels, etc., following a single administration of a dosage form to each patient.

“Ascending plasma concentration” means a drug plasma concentration profile over about the first 12 to 24 hours following initial dosing, wherein the profile shows an increase to a maximum concentration, wherein said maximum occurs more than about 9 hours following the initial dose, preferably, more than about 10 hours following initial dose, more preferably, more than about 12 hours after dose.

Persons of skill in the art will appreciate that blood plasma drug concentrations obtained in individual subjects will vary due to interpatient variability in the many parameters affecting drug absorption, distribution, metabolism and excretion. For this reason, unless otherwise indicated, when a drug plasma concentration is listed, the value listed is the calculated mean value based on values obtained from a groups of subjects tested.

“Ascending rate of release” or “ascending release rate” means a rate of release wherein the amount of drug released from a dosage form as a function of time increases over a period of time, preferably continuously and gradually. Preferably, the rate of drug released as a function of time increases in a steady (rather than step-wise) manner. More preferably, an ascending rate of release may be characterized as follows. The rate of release as a function of time for a dosage form is measured and plotted as % drug release versus time or as milligrams of drug released/hour versus time. An ascending rate of release is preferably characterized by an average rate (expressed in mg of drug per hour) wherein the rate within a given two hour span is higher as compared with the previous two hour time span, over the period of time of about 2 hours to about 12 hours, preferably, about 2 hours to about 18 hours, more preferably about 4 hours to about 12 hours, more preferably still, about 4 hours to about 18 hours. Preferably, the increase in average rate is gradual such that less than about 30% of the dose is delivered during any 2 hour interval, more preferably, less than about 25% of the dose is delivered during any 2 hour interval. Preferably, the ascending release rate is maintained until at least about 50%, more preferably until at least about 75% of the drug in the dosage form has been released.

In other preferably embodiments, ascending rates of release may be defined with reference to specific release rates measured at specified times following administration of the dosage form in question. Preferably such release rates are determined in vitro.

“Benzisoxazole derivative” or “drug” means risperidone and/or pharmaceutically acceptable salt(s) thereof, or paliperidone and/or pharmaceutically acceptable salt(s) thereof. “Benzisoxazole derivative and pharmacologically active metabolites thereof taken together” or “active moiety” means the sum of risperidone and/or its pharmaceutically acceptable salt(s) thereof, and paliperidone and/or its pharmaceutically acceptable salt(s) thereof.

“C” means the concentration of drug in blood plasma, or serum, of a subject, generally expressed as mass per unit volume, typically nanograms per milliliter. For convenience, this concentration may be referred to herein as “drug plasma concentration”, “plasma drug concentration” or “plasma concentration”. The plasma drug concentration at any time following drug administration is referenced as Ctime, as in C9h or C24h, etc. A maximum plasma concentration obtained following administration of a dosage form obtained directly from the experimental data without interpolation is referred to as Cmax. The average or mean plasma concentration obtained during a period of interest is referred to as Cavg or Cmean. “Mean, single dose, maximum plasma concentration Cmax” means the mean Cmax obtained over several patients or multiple administrations to the same patient with sufficient washout in between dosings to allow drug levels to subside to pre-dose levels, etc., etc., following a single administration of a dosage form to each patient.

“Dosage form” means a benzisoxazole derivative in a medium, carrier, vehicle, or device suitable for administration to a patient. “Oral dosage form” means a dosage form suitable for oral administration.

“Dose” means a unit of drug. Conventionally, a dose is provided as a dosage form. Doses may be administered to patients according to a variety of dosing regimens. Common dosing regimens include once daily orally (qd), twice daily orally (bid), and thrice daily orally (tid).

“Flat plasma curve” means a plasma concentration curve that reaches and maintains a substantially constant value after a defined period of time following administration of a dosage form according to the invention.

“Immediate-release dosage form” means a dosage form that releases greater than or equal to about 80% of the drug in less than or equal to about 1 hour following administration of the dosage form to a patient.

“Oral sustained release dosing structure” means a structure suitable for oral administration to a patient comprising one or more benzisoxazole derivatives, wherein the structure operates to sustainably release the benzisoxazole derivative(s). “Osmotic oral sustained release dosing structure” means an oral sustained release dosing structure wherein the structure operates via an osmotic mechanism to sustainably release the benzisoxazole derivative(s).

“Patient” means an animal, preferably a mammal, more preferably a human, in need of therapeutic intervention.

“Pharmaceutically acceptable salt” means any salt whose anion does not contribute significantly to the toxicity or pharmacological activity of the salt, and, as such, they are the pharmacological equivalents of the base of the benzisoxazole derivative. Suitable pharmaceutically acceptable salts include acid addition salts which may, for example, be formed by reacting the drug compound with a suitable pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid.

Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

“Pharmacologically active metabolites” means pharmacologically active metabolites of benzisoxazole derivatives.

“Plasma drug concentration curve” or “drug plasma concentration curve”, or “plasma concentration curve” or “plasma profile” or “plasma concentration profile” refer to the curve obtained by plotting plasma drug concentration or drug plasma concentration, or plasma concentration versus time. Usually, the convention is that the zero point on the time scale (conventionally on the x axis) is the time of administration of the drug or dosage form comprising the drug to a patient.

“Prolonged period of time” means a continuous period of time of greater than about 2 hours, preferably, greater than about 4 hours, more preferably, greater than about 8 hours, more preferably greater than about 10 hours, more preferably still, greater than about 14 hours, most preferably, greater than about 14 hours and up to about 24 hours.

“Rate of release” or “release rate” means to the quantity of benzisoxazole derivative released from a dosage form per unit time, e.g., milligrams of drug released per hour (mg/hr). Drug release rates for dosage forms may be measured as an in vitro rate of drug release, i.e., a quantity of drug released from the dosage form per unit time measured under appropriate conditions and in a suitable fluid.

The release rates referred to herein are determined by placing a dosage form to be tested in de-ionized water in metal coil or metal cage sample holders attached to a USP Type VII bath indexer in a constant temperature water bath at 37° C. Aliquots of the release rate solutions, collected at pre-set intervals, are then injected into a chromatographic system fitted with an ultraviolet or refractive index detector to quantify the amounts of drug released during the testing intervals.

As used herein a drug release rate obtained at a specified time refers to the in vitro release rate obtained at the specified time following implementation of the release rate test. The time at which a specified percentage of the drug within a dosage form has been released from said dosage form may be referred to as the “Tx” value, where “x” is the percent of drug that has been released. For example, a commonly used reference measurement for evaluating drug release from dosage forms is the time at which 70% of drug within the dosage form has been released. This measurement is referred to as the “T70” for the dosage form.

“Relative bioavailability” means

AUC_{inf} for inventive dosage form/AUC_{inf} for immediate release dosage form;

wherein both dosage forms comprise the same or substantially the same amount of drug, expressed in units of mass.

“Steady state plasma concentration” means the condition in which the amount of drug present in the plasma of a patient does not vary significantly over a prolonged period of time. A pattern of drug accumulation following continuous administration of a constant dose and dosage form at constant dosing intervals eventually achieves a “steady-state” where the plasma concentration peaks and plasma concentration troughs are essentially identical from dosing interval to dosing interval. As used herein, the steady-state maximal (peak) plasma drug concentration obtained directly from the experimental data without interpolation is referenced as Cₘₐₓ₋ₛₛ and the steady-state minimal (trough) plasma drug concentration obtained directly from the experimental data without interpolation is referenced as Cₘₐₓ₋ₛₛ. The times following drug administration at which the steady-state peak plasma and trough drug concentrations occur are referenced as the Tₘₐₓ₋ₛₛ and the Tₘᵢₙ₋ₛₛ, respectively. Typically these values are reported as a mean obtained over several patients or multiple administrations to the same patient, etc., once a steady state plasma concentration has been achieved.

“Sustained release” or “sustainably releasing” means continuous release or continuously releasing of a drug or a dose of a drug over a prolonged period of time.

“Mean, single dose, time to maximum plasma concentration Tmax” is the mean time elapsed from administration to a patient of a dosage form comprising a drug to the time at which the Cmax for that drug is obtained over several patients or multiple administrations to the same patient to the same patient with sufficient washout in between dosings to allow drug levels to subside to pre-dose levels, etc., following a single administration of the dosage form to each patient, and obtained directly from the experimental data without interpolation.

“Therapeutically effective amount” means that amount of drug that elicits the biological or medicinal response in a tissue system, animal or human that is being sought by a researcher, veterinarian, medical doctor or other clinician, which includes alleviation of the symptoms of the disease or disorder being treated.

“Zero order rate of release” or “zero order release rate” means a rate of release wherein the amount of drug released as a function of time is substantially constant. In other words, the dosage form exhibits zero order or substantially zero order release kinetics. More particularly, the rate of release of drug as a function of time shall vary by less than about 30%, preferably, less than about 20%, more preferably, less than about 10%, most preferably, less than about 5%, wherein the measurement is taken over the period of time wherein the cumulative release is between about 25% and about 75%, preferably, between about 25% and about 90%.

### III. Plasma Profiles

As discussed above, physicians have noted certain side effects associated with risperidone administration, which the inventors have investigated and found to be applicable to other benzisoxazole derivatives, such as paliperidone. These side effects include, but are not limited to orthostatic hypotension, seizures, dysphagia, and hyperprolactinemia. Physicians have determined that certain of these side effects can be alleviated by titration to the efficacious dose over several days. In effect, the patient develops a tolerance to the side effects over time. While this titration approach reduces the side effects of risperidone to the patient, it also reduced the utility of the drug, because of the effect of drug holidays.

Drug holidays arise when patients at a stabilized efficacious dose level may intentionally or unintentionally skip a dose. These drug holiday periods can be intentional, such as for medical reasons, or sometimes due to patient inadvertence, poor compliance, etc. In these patients there is a potential of significant orthostatic hypotension, or other side effects, once they resume, without titration, the stable dose that they were on prior to the drug holiday. Alternatively, patients may resume therapy with a subtherapeutic dose and titrate up again. This means that the subject would not receive the original therapeutic benefit for several more days or weeks after their drug holiday period. These problems are significant for a psychotic patient population who, for a variety of reasons, may be less compliant than other patient populations and more vulnerable to the effects of sub-therapeutic dosing.

Accordingly, while titration does address a desire to reduce side effects of risperidone, and potentially other benzisoxazole derivatives, it can cause problems in typical usage scenarios. Substantially reducing or substantially eliminating titration is a reasonable goal for improving dosing of benzisoxazole derivatives. Clinical data currently available in the art, however, does not establish how benzisoxazole derivative side effects, particularly orthostatic hypotension, can be reduced enough at clinically meaningful dosages so as to substantially reduce or substantially eliminate the need for titration while maintaining enough drug in the body (as measured by plasma concentration) to insure efficacy.

The inventors have discovered that it is possible to adjust dosing of benzisoxazole derivatives such that benzisoxazole derivative side effects, particularly orthostatic hypotension, can be reduced enough at clinically meaningful dosages so as to substantially reduce or substantially eliminate the need for titration while maintaining enough drug in the body (as measured by plasma concentration) to insure efficacy.

For instance, in Example 2 below, dosage forms that provided ascending plasma concentration provided superior performance to classic immediate release dosing. In particular, ascending plasma concentration dosage forms produced results suggesting that tolerance to orthostatic hypotension could develop within a day, when the plasma concentrations rise slowly. Similarly, with prolactin elevation, the ascending plasma concentration dosage forms had the lowest Cmax value on day 1 compared with the Flat and IR treatments despite the higher dose. Accordingly, the ascending plasma concentration profile is unexpectedly better tolerated with respect to orthostatic hypotension and to result in less prolactin elevation than either the Flat or IR plasma profiles.

In Example 3, the flat plasma profile produced the lowest incidence of orthostatic hypotension. Further, the ascending plasma concentration treatment resulted in an incidence of orthostatic hypotension similar to the IR treatment on Day 1, despite a 75% higher dose. The ascending plasma concentration treatment also resulted in less prolactin elevation as compared to the other treatments.

The results shown in Example 4 further illustrate that modified dosing, particularly ascending plasma concentration treatments, could reduce orthostatic hypotension and reduce prolactin levels. The results shown in Example 10 suggest that dosing profiles with a longer mean, single dose, time to maximum plasma concentration Tmax can provide greater improvements over dosing profiles with shorter Tmax. In an embodiment, Tmax≧about 9 hours.

Finally, in Example 14, 12 mg (6×2 mg) OROS® paliperidone dosage forms were shown to be noninferior to 2 mg IR risperidone with respect to initial orthostatic tolerability in subjects with schizophrenia. The safety profile of 12 mg (6×2 mg) ER OROS paliperidone once daily for 5 to 6 days was similar to that of 2 mg IR risperidone on Day 1 followed by 4 mg IR risperidone on Days 2 to 6.

The results above, particularly in Example 12, provide evidence that a dosage form according to the invention can substantially reduce or substantially eliminate the need for titration while maintaining the patient's psychiatric condition. The inventive pharmacokinetic profiles that accomplish this result, and dosage form release profiles that provide the inventive pharmacokinetic profiles, have not been demonstrated previously in the art. Accordingly, the contribution of this knowledge supports patentability over the prior art.

Further, as noted above, the advantages of the present invention include reductions with regard to dizziness, fainting, orthostatic hypertension, drop in blood pressure, heart rate, extrapyramidal disorder, and tachycardia. An advantage of the present invention is that it provides for a new use of benzisoxazole derivatives: stabilization of a patient's psychiatric condition while reducing, substantially reducing, eliminating or substantially eliminating the particular side effects/conditions noted above.

In an embodiment, the invention provides for a reduction, substantial reduction, elimination or substantial elimination in the number of patients needing dose titration to manage orthostatic hypotension with stabilization of the patients' psychiatric condition by about 30% or more compared to the patients taking an immediate release dosage form containing one-half the amount (preferably measured in mass) of the drug as the inventive dosage form, wherein the amount of drug in the inventive dosage form is adjusted for its relative bioavailability. More preferably, the invention provides for a reduction, substantial reduction, elimination or substantial elimination in the number of patients needing dose titration to manage orthostatic hypotension with stabilization of the patients' psychiatric condition by about 40% or more compared to the patients taking an immediate release dosage form containing one-half the amount (preferably measured in mass) of the drug as the inventive dosage form, wherein the amount of drug in the inventive dosage form is adjusted for its relative bioavailability. Still more preferably, the invention provides for a reduction, substantial reduction, elimination or substantial elimination in the number of patients needing dose titration to manage orthostatic hypotension with stabilization of the patients' psychiatric condition by about 50% or more compared to the patients taking an immediate release dosage form containing one-half the amount (preferably measured in mass) of the drug as the inventive dosage form, wherein the amount of drug in the inventive dosage form is adjusted for its relative bioavailability. This new use is clinically meaningful for the reasons noted elsewhere herein.

Next, Example 1 provides disclosure of a difference in bioavailability of benzisoxazole derivatives in the colon versus the upper gastrointestinal tract (“upper GI”). The recognition of this difference is significant because it impacts the selection of inventive controlled release dosage forms release rates. An objective of release rate selection is to achieve the inventive pharmacokinetic profiles. Knowledge of colonic absorption of benzisoxazole derivatives is important in determining the required release rates in controlled release dosage forms to achieve the inventive pharmacokinetic profiles. This is because controlled release dosage forms may deliver a drug in the lower GI tract (e.g. colon). Accordingly, the contribution of this knowledge supports patentability over the prior art. The present invention provides for a variety of embodiments, both exemplified and non-exemplified, that embody this information to achieve methods, uses, and dosage forms according to the invention.

### IV. Dosage Forms

In embodiments, the inventive sustained release dosage forms are formulated into dosage forms administrable to patients in need thereof. Sustained release dosage forms and methods of treatment using the sustained release dosage forms will now be described. It will be appreciated that the sustained release dosage forms described below are merely exemplary.

A variety of sustained release dosage forms are suitable for use in the present invention. In certain embodiments, the dosage form is orally administrable and is sized and shaped as a conventional tablet or capsule. Orally administrable dosage forms may be manufactured according to one of various different approaches. For example, the dosage form may be manufactured as a diffusion system, such as a reservoir device or matrix device, a dissolution system, such as encapsulated dissolution systems (including, for example, “tiny time pills”, and beads) and matrix dissolution systems, and combination diffusion/dissolution systems and ion-exchange resin systems, as described in Pharmaceutical Sciences, Remington, 18th Ed., pp. 1676-1686 (1990), Mack Publishing Co.; The Pharmaceutical and Clinical Pharmacokinetics, 3rd Ed., pp. 1-28 (1984), Lea and Febreger, Philadelphia.

Osmotic dosage forms in general utilize osmotic pressure to generate a driving force for imbibing fluid into a compartment formed, at least in part, by a semipermeable membrane that permits free diffusion of fluid but not drug or osmotic agent(s), if present. A significant advantage to osmotic systems is that operation is pH-independent and thus continues at the osmotically determined rate throughout an extended time period even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release 35 (1995) 1-21, incorporated by reference herein. U.S. Pat. Nos. 3,845,770; 3,916,899; 3,995,631; 4,008,719; 4,111,202; 4,160,020; 4,327,725; 4,578,075; 4,681,583; 5,019,397; and 5,156,850 disclose osmotic devices for the continuous dispensing of active agent.

Osmotic dosage forms in which a drug composition is delivered as a slurry, suspension or solution from a small exit orifice by the action of an expandable layer are disclosed in U.S. Pat. Nos. 5,633,011; 5,190,765; 5,252,338; 5,620,705; 4,931,285; 5,006,346; 5,024,842; and 5,160,743, which are incorporated herein by reference. Typical devices include an expandable push layer and a drug layer surrounded by a semipermeable membrane. In certain instances, the drug layer is provided with a subcoat to delay release of the drug composition to the environment of use or to form an annealed coating in conjunction with the semipermeable membrane.

A dosage form exhibiting substantially ascending release rate profile is Concerta® marketed by McNeil Consumer Healthcare and ALZA Pharmaceuticals. Physicians' Desk Reference, Thompson Healthcare, 56th Ed., pp. 1998-2001 (2002). The Concerta® product, which contains methylphenidate as active agent, however, only delivers active agent at a substantially ascending rate of release for up to about 8 hours. Patent applications relating to Concerta® include published PCT Patent Application No. WO99/62496A1. This patent application discloses the substantially ascending release rate profile related to Concerta® for delivery over about 8 hours for once-a-day dosing.

Related ascending release rate profile patent applications include published PCT Patent Application No. WO98/14168; WO98/23263; WO 98/06380A2,U.S. 2001/0012847A1 and U.S. 2002/0035357A1, which disclose an ascending release of active agent, including methylphenidate and pseudoephedrine, for up to about 8 hours. Still other applications relating to providing increasing rate of release delivery profile include WO 01/52819A1, which discloses extended release of nifedipine and WO 01/37813A2, which discloses at least a four layer preparation to provide controlled release.

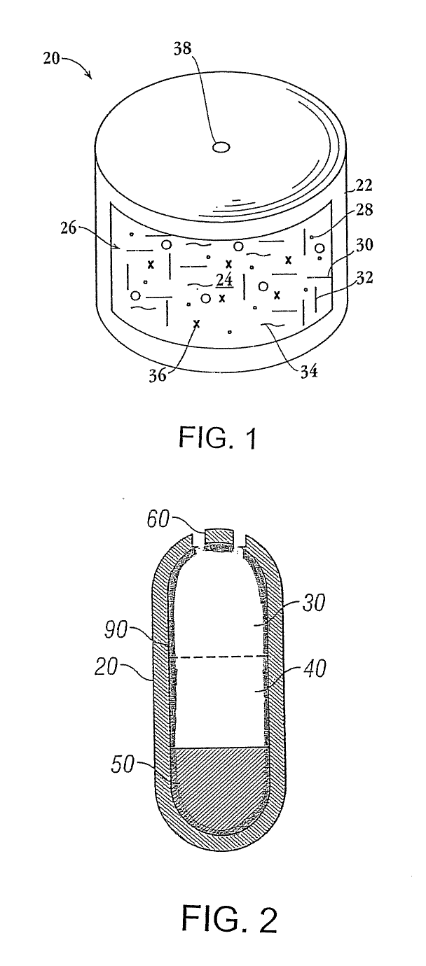

An exemplary dosage form, referred to in the art as an elementary osmotic pump dosage form, is shown in FIG. 1. Dosage form 20, shown in a cutaway view, is also referred to as an elementary osmotic pump, and is comprised of a semi-permeable wall 22 that surrounds and encloses an internal compartment 24. The internal compartment contains a single component layer referred to herein as a drug layer 26, comprising an inventive substance 28 in an admixture with selected excipients. The excipients are adapted to provide an osmotic activity gradient for attracting fluid from an external environment through wall 22 and for forming a deliverable complex formulation upon imbibition of fluid. The excipients may include a suitable suspending agent, also referred to herein as drug carrier 30, a binder 32, a lubricant 34, and an osmotically active agent referred to as an osmagent 36. Exemplary materials useful for these components can be found disclosed throughout the present application.

Semi-permeable wall 22 of the osmotic dosage form is permeable to the passage of an external fluid, such as water and biological fluids, but is substantially impermeable to the passage of components in the internal compartment. Materials useful for forming the wall are essentially nonerodible and are substantially insoluble in biological fluids during the life of the dosage form. Representative polymers for forming the semi-permeable wall include homopolymers and copolymers, such as, cellulose esters, cellulose ethers, and cellulose ester-ethers. Flux-regulating agents can be admixed with the wall-forming material to modulate the fluid permeability of the wall. For example, agents that produce a marked increase in permeability to fluid such as water are often essentially hydrophilic, while those that produce a marked permeability decrease to water are essentially hydrophobic. Exemplary flux regulating agents include polyhydric alcohols, polyalkylene glycols, polyalkylenediols, polyesters of alkylene glycols, and the like.

In operation, the osmotic gradient across wall 22 due to the presence of osmotically-active agents causes gastric fluid to be imbibed through the wall, swelling of the drug layer, and formation of a deliverable complex formulation (e.g., a solution, suspension, slurry or other flowable composition) within the internal compartment. The deliverable inventive substance formulation is released through an exit 38 as fluid continues to enter the internal compartment. Even as drug formulation is released from the dosage form, fluid continues to be drawn into the internal compartment, thereby driving continued release. In this manner, the inventive substance is released in a sustained and continuous manner over an extended time period.

FIG. 2 illustrates certain inventive embodiments of sustained release dosage forms. Dosage forms of this type are described in detail in U.S. Pat. Nos. 4,612,008; 5,082,668; and 5,091,190; and are further described below

FIG. 2 shows an embodiment of one type of sustained release dosage form, namely the osmotic sustained release dosage form. First drug layer 30 comprises osmotically active components, and a lower amount of active agent than in second drug layer 40. The osmotically active component(s) in the first component drug layer comprises an osmagent such as salt and one or more osmopolymer(s) having relatively small molecular weights which exhibit swelling as fluid is imbibed such that release of these osmopolymers through exit 60 occurs similar to that of drug layer 40. Additional excipients such as binders, lubricants, antioxidants and colorants may also be included in first drug layer 30.

Second drug layer 40 comprises active agent in an admixture with selected excipients adapted to provide an osmotic activity gradient for driving fluid from an external environment through membrane 20 and for forming a deliverable drug formulation upon imbibition of fluid. The excipients may include a suitable suspending agent, also referred to herein as a drug carrier, but no osmotically active agent, “osmagent,” such as salt, sodium chloride. It has been discovered that the omission of salt from this second drug layer, which contains a higher proportion of the overall drug in the dosage form, in combination with the salt in the first drug layer, provides an improved ascending rate of release creating a longer duration of ascending rate.

Drug layer 40 has a higher concentration of the drug than does drug layer 30. The ratio of the concentration of drug in the first drug layer 30 to the concentration of drug in the second drug layer 40 is maintained at less than 1 and preferably less than or equal to about 0.43 to provide the desired substantially ascending rate of release.

Drug layer 40 may also comprise other excipients such as lubricants, binders, etc.

Drug layer 40, as with drug layer 30, further comprises a hydrophilic polymer carrier. The hydrophilic polymer provides a particle in the drug composition that contributes to the controlled delivery of the active drug. Representative examples of these polymers are poly(alkylene oxide) of 100,000 to 750,000 number-average molecular weight, including poly(ethylene oxide), poly(methylene oxide), poly(butylene oxide) and poly(hexylene oxide); and a poly(carboxymethylcellulose) of 40,000 to 400,000 number-average molecular weight, represented by poly(alkali carboxymethylcellulose), poly(sodium carboxymethylcellulose), poly(potassium carboxymethylcellulose) and poly(lithium carboxymethylcellulose). Drug layer 40 can further comprise a hydroxypropylalkylcellulose of 9,200 to 125,000 number-average molecular weight for enhancing the delivery properties of the dosage form as represented by hydroxypropylethylcellulose, hydroxypropylmethylcellulose, hydroxypropylbutylcellulose and hydroxypropylpentylcellulose; and a poly(vinylpyrrolidone) of 7,000 to 75,000 number-average molecular weight for enhancing the flow properties of the dosage form. Preferred among these polymers are the poly(ethylene oxide) of 100,000-300,000 number average molecular weight. Carriers that erode in the gastric environment, i.e., bioerodible carriers, are especially preferred.

Other carriers that may be incorporated into drug layer 40, and/or drug layer 30, include carbohydrates that exhibit sufficient osmotic activity to be used alone or with other osmagents. Such carbohydrates comprise monosaccharides, disaccharides and polysaccharides. Representative examples include maltodextrins (i.e., glucose polymers produced by the hydrolysis of corn starch) and the sugars comprising lactose, glucose, raffinose, sucrose, mannitol, sorbitol, and the like. Preferred maltodextrins are those having a dextrose equivalence (DE) of 20 or less, preferably with a DE ranging from about 4 to about 20, and often 9-20. Maltodextrin having a DE of 9-12 has been found to be useful.

Drug layer 40 and drug layer 30 typically will be a substantially dry, <1% water by weight, composition formed by compression of the carrier, the drug, and other excipients as one layer.

Drug layer 40 may be formed from particles by comminution that produces the size of the drug and the size of the accompanying polymer used in the fabrication of the drug layer, typically as a core containing the compound, according to the mode and the manner of the invention. The means for producing particles include granulation, spray drying, sieving, lyophilization, crushing, grinding, jet milling, micronizing and chopping to produce the intended micron particle size. The process can be performed by size reduction equipment, such as a micropulverizer mill, a fluid energy grinding mill, a grinding mill, a roller mill, a hammer mill, an attrition mill, a chaser mill, a ball mill, a vibrating ball mill, an impact pulverizer mill, a centrifugal pulverizer, a coarse crusher and a fine crusher. The size of the particle can be ascertained by screening, including a grizzly screen, a flat screen, a vibrating screen, a revolving screen, a shaking screen, an oscillating screen and a reciprocating screen. The processes and equipment for preparing drug and carrier particles are disclosed in Pharmaceutical Sciences, Remington, 17th Ed., pp. 1585-1594 (1985); Chemical Engineers Handbook, Perry, 6th Ed., pp. 21-13 to 21-19 (1984); Journal of Pharmaceutical Sciences, Parrot, Vol. 61, No. 6, pp. 813-829 (1974); and Chemical Engineer, Nixon, pp. 94-103 (1990).

First drug layer 30 comprises active agent in an admixture with selected excipients adapted to provide an osmotic activity gradient for driving fluid from an external environment through membrane 20 and for forming a deliverable drug formulation upon imbibition of fluid. The excipients may include a suitable suspending agent, also referred to herein as a drug carrier, and an osmotically active agent, i.e., an “osmagent,” such as salt. Other excipients such as lubricants, binders, etc. may also be included. It has been surprisingly found that when first component drug layer 30 comprises an osmotically active component, and a lower amount of active drug than in second component drug layer 40, an improved ascending rate of release can be created that provides a longer duration of ascending rate. Additionally, with the low doses of paliperidone delivered from a dosage form, and the low amount of that total in the first drug layer 30, the addition of salt has been found to provide a consistent predetermined release rate providing a substantially ascending rate of release over 20 hours.

The osmotically active component in the first drug layer typically comprises an osmagent and one or more osmopolymer(s) having relatively small molecular weights which exhibit swelling as fluid is imbibed such that release of these osmopolymers through exit 60 occurs similar to that of drug layer 40.

The ratio of drug concentration between the first drug layer and the second drug layer alters the release rate profile. Release rate profile is calculated as the difference between the maximum release rate and the release rate achieved at the first time point after start-up (for example, at 6 hours), divided by the average release rate between the two data points.

Drug layer 30 and drug layer 40 may optionally contain surfactants and disintegrants in both drug layers. Exemplary of the surfactants are those having an HLB value of about 10-25, such as polyethylene glycol 400 monostearate, polyoxyethylene-4-sorbitan monolaurate, polyoxyethylene-20-sorbitan monooleate, polyoxyethylene-20-sorbitan monopalmitate, polyoxyethylene-20-monolaurate, polyoxyethylene-40-stearate, sodium oleate and the like.

Disintegrants may be selected from starches, clays, celluloses, algins and gums and crosslinked starches, celluloses and polymers. Representative disintegrants include corn starch, potato starch, croscarmelose, crospovidone, sodium starch glycolate, Veegum HV, methylcellulose, agar, bentonite, carboxymethylcellulose, alginic acid, guar gum and the like.

Wall 20 is formed to be permeable to the passage of an external fluid, such as water and biological fluids, and is substantially impermeable to the passage of paliperidone, osmagent, osmopolymer and the like. As such, it is semipermeable. The selectively semipermeable compositions used for forming wall 20 are essentially nonerodible and substantially insoluble in biological fluids during the life of the dosage form.

Representative polymers for forming wall 20 comprise semipermeable homopolymers, semipermeable copolymers, and the like. In one presently preferred embodiment, the compositions can comprise cellulose esters, cellulose ethers, and cellulose ester-ethers. The cellulosic polymers typically have a degree of substitution, “D.S.”, on their anhydroglucose unit from greater than 0 up to 3 inclusive. By degree of substitution is meant the average number of hydroxyl groups originally present on the anhydroglucose unit that are replaced by a substituting group, or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkylsulfamate, semipermeable polymer forming groups, and the like. The semipermeable compositions typically include a member selected from the group consisting of cellulose acylate, cellulose diacylate, cellulose triacylate, cellulose triacetate, cellulose acetate, cellulose diacetate, cellulose triacetate, mono-, di- and tri-cellulose alkanylates, mono-, di-, and tri-alkenylates, mono-, di-, and tri-aroylates, and the like.

Exemplary polymers can include, for example, cellulose acetate have a D.S. of 1.8 to 2.3 and an acetyl content of 32 to 39.9%; cellulose diacetate having a D.S. of 1 to 2 and an acetyl content of 21 to 35%, cellulose triacetate having a D.S. of 2 to 3 and an acetyl content of 34 to 44.8%, and the like. More specific cellulosic polymers include cellulose propionate having a D.S. of 1.8 and a propionyl content of 38.5%; cellulose acetate propionate having an acetyl content of 1.5 to 7% and an acetyl content of 39 to 42%; cellulose acetate propionate having an acetyl content of 2.5 to 3%, an average propionyl content of 39.2 to 45%, and a hydroxyl content of 2.8 to 5.4%; cellulose acetate butyrate having a D.S. of 1.8, an acetyl content of 13 to 15%, and a butyryl content of 34 to 39%; cellulose acetate butyrate having an acetyl content of 2 to 29%, a butyryl content of 17 to 53%, and a hydroxyl content of 0.5 to 4.7%; cellulose triacylates having a D.S. of 2.6 to 3 such as cellulose trivalerate, cellulose trilamate, cellulose tripalmitate, cellulose trioctanoate, and cellulose tripropionate; cellulose diesters having a D.S. of 2.2 to 2.6 such as cellulose disuccinate, cellulose dipalmitate, cellulose dioctanoate, cellulose dicarpylate, and the like; mixed cellulose esters such as cellulose acetate valerate, cellulose acetate succinate, cellulose propionate succinate, cellulose acetate octanoate, cellulose valerate palmitate, cellulose acetate heptonate, and the like. Semipermeable polymers are known in U.S. Pat. No. 4,077,407 and they can be synthesized by procedures described in Encyclopedia of Polymer Science and Technology, Vol. 3, pages 325 to 354, 1964, published by Interscience Publishers, Inc., New York.

Additional semipermeable polymers for forming the semipermeable wall can comprise, for example, cellulose acetaldehyde dimethyl acetate; cellulose acetate ethylcarbamate; cellulose acetate methylcarbamate; cellulose dimethylaminoacetate; semipermeable polyamide; semipermeable polyurethanes; semipermeable sulfonated polystyrenes; cross-linked selectively semipermeable polymers formed by the coprecipitation of a polyanion and a polycation as disclosed in U.S. Pat. Nos. 3,173,876; 3,276,586; 3,541,005; 3,541,006; and 3,546,142; semipermeable polymers as disclosed in U.S. Pat. No. 3,133,132; semipermeable polystyrene derivatives; semipermeable poly (sodium styrenesulfonate); semipermeable poly(vinylbenzyltremethylammonium chloride); semipermeable polymers, exhibiting a fluid permeability of 10-5 to 10-2 (cc. mil/cm hr·atm) expressed as per atmosphere of hydrostatic or osmotic pressure differences across a semipermeable wall. The polymers are known to the art in U.S. Pat. Nos. 3,845,770; 3,916,899; and 4,160,020; and in Handbook of Common Polymers, by Scott, J. R., and Roff, W. J., 1971, published by CRC Press, Cleveland. Ohio.

Wall 20 may also comprise a flux-regulating agent. The flux regulating agent is a compound added to assist in regulating the fluid permeability or flux through the wall 20. The flux regulating agent can be a flux enhancing agent or a decreasing agent. The agent can be preselected to increase or decrease the liquid flux. Agents that produce a marked increase in permeability to fluids such as water are often essentially hydrophilic, while those that produce a marked decrease to fluids such as water are essentially hydrophobic. The amount of regulator in wall 20 when incorporated therein generally is from about 0.01% to 20% by weight or more. The flux regulator agents in one embodiment that increase flux include, for example, polyhydric alcohols, polyalkylene glycols, polyalkylenediols, polyesters of alkylene glycols, and the like. Typical flux enhancers include polyethylene glycol 300, 400, 600, 1500, 4000, 6000, poly(ethylene glycol-co-propylene glycol), and the like; low molecular weight gylcols such as polypropylene glycol, polybutylene glycol and polyamylene glycol: the polyalkylenediols such as poly(1,3-propanediol), poly(1,4-butanediol), poly(1,6-hexanediol), and the like; aliphatic diols such as 1,3-butylene glycol, 1,4-pentamethylene glycol, 1,4-hexamethylene glycol, and the like; alkylene triols such as glycerine, 1,2,3-butanetriol, 1,2,4-hexanetriol, 1,3,6-hexanetriol and the like; esters such as ethylene glycol dipropionate, ethylene glycol butyrate, butylene glucol dipropionate, glycerol acetate esters, and the like. Representative flux decreasing agents include, for example, phthalates substituted with an alkyl or alkoxy or with both an alkyl and alkoxy group such as diethyl phthalate, dimethoxyethyl phthalate, dimethyl phthalate, and [di(2-ethylhexyl)phthalate], aryl phthalates such as triphenyl phthalate, and butyl benzyl phthalate; insoluble salts such as calcium sulphate, barium sulphate, calcium phosphate, and the like; insoluble oxides such as titanium oxide; polymers in powder, granule and like form such as polystyrene, polymethylmethacrylate, polycarbonate, and polysulfone; esters such as citric acid esters esterfied with long chain alkyl groups; inert and substantially water impermeable fillers; resins compatible with cellulose based wall forming materials, and the like.

Other materials that can be used to form wall 20 for imparting flexibility and elongation properties to the wall, for making the wall less-to-nonbrittle and to render tear strength, include, for example, phthalate plasticizers such as dibenzyl phthalate, dihexyl phthalate, butyl octyl phthalate, straight chain phthalates of six to eleven carbons, di-isononyl phthalte, di-isodecyl phthalate, and the like. The plasticizers include nonphthalates such as triacetin, dioctyl azelate, epoxidized tallate, tri-isoctyl trimellitate, tri-isononyl trimellitate, sucrose acetate isobutyrate, epoxidized soybean oil, and the like. The amount of plasticizer in a wall when incorporated therein is about 0.01% to 20% weight, or higher.

Push layer 50 comprises an expandable layer in contacting layered arrangement with the second component drug layer 40 as illustrated in FIG. 2. Push layer 50 comprises a polymer that imbibes an aqueous or biological fluid and swells to push the drug composition through the exit of the device.

The expandable layer comprises in one embodiment a hydroactivated composition that swells in the presence of water, such as that present in gastric fluids. Conveniently, it can comprise an osmotic composition comprising an osmotic solute that exhibits an osmotic pressure gradient across the semipermeable layer against an external fluid present in the environment of use. In another embodiment, the hydro-activated layer comprises a hydrogel that imbibes and/or absorbs fluid into the layer through the outer semipermeable wall. The semipermeable wall is non-toxic. It maintains its physical and chemical integrity during operation and it is essentially free of interaction with the expandable layer.

The expandable layer in one preferred embodiment comprises a hydroactive layer comprising a hydrophilic polymer, also known as osmopolymers. The osmopolymers exhibit fluid imbibition properties. The osmopolymers are swellable, hydrophilic polymers, which osmopolymers interact with water and biological aqueous fluids and swell or expand to an equilibrium state. The osmopolymers exhibit the ability to swell in water and biological fluids and retain a significant portion of the imbibed fluid within the polymer structure. The osmopolymers swell or expand to a very high degree, usually exhibiting a 2 to 50 fold volume increase. The osmopolymers can be non-cross-linked or cross-linked. The swellable, hydrophilic polymers are in one embodiment lightly cross-linked, such cross-links being formed by covalent or ionic bonds or residue crystalline regions after swelling. The osmopolymers can be of plant, animal or synthetic origin.

The osmopolymers are hydrophilic polymers. Hydrophilic polymers suitable for the present purpose include poly(hydroxy-alkyl methacrylate) having a molecular weight of from 30,000 to 5,000,000; poly(vinylpyrrolidone) having a molecular weight of from 10,000 to 360,000; anionic and cationic hydrogels; polyelectrolytes complexes; poly(vinyl alcohol) having a low acetate residual, cross-linked with glyoxal, formaldehyde, or glutaraldehyde and having a degree of polymerization of from 200 to 30,000; a mixture of methyl cellulose, cross-linked agar and carboxymethyl cellulose; a mixture of hydroxypropyl methylcellulose and sodium carboxymethylcellulose; a mixture of hydroxypropyl ethylcellulose and sodium carboxymethyl cellulose, a mixture of sodium carboxymethylcellulose and methylcellulose, sodium carboxymethylcellulose; potassium carboxymethylcellulose; a water insoluble, water swellable copolymer formed from a dispersion of finely divided copolymer of maleic anhydride with styrene, ethylene, propylene, butylene or isobutylene crosslinked with from 0.001 to about 0.5 moles of saturated cross-linking agent per mole of maleic anhydride per copolymer; water swellable polymers of N-vinyl lactams; polyoxyethylene-polyoxypropylene gel; carob gum; polyacrylic gel; polyester gel; polyuria gel; polyether gel, polyamide gel; polycellulosic gel; polygum gel; initially dry hydrogels that imbibe and absorb water which penetrates the glassy hydrogel and lowers its glass temperature; and the like.

Representative of other osmopolymers are polymers that form hydrogels such as Carbopol™. acidic carboxypolymer, a polymer of acrylic acid cross-linked with a polyallyl sucrose, also known as carboxypolymethylene, and carboxyvinyl polymer having a molecular weight of 250,000 to 4,000,000; CYANAMER® polyacrylamides; cross-linked water swellable indenemaleic anhydride polymers; GOOD RITE® polyacrylic acid having a molecular weight of 80,000 to 200,000; Polyox™ polyethylene oxide polymer having a molecular weight of 100,000 to 5,000,000 and higher; starch graft copolymers; AQUA KEEPS® acrylate polymer polysaccharides composed of condensed glucose units such as diester cross-linked polygluran; and the like. Representative polymers that form hydrogels are known to the prior art in U.S. Pat. No. 3,865,108; U.S. Pat. No. 4,002,173; U.S. Pat. No. 4,207,893; and in Handbook of Common Polymers, by Scott and Roff, published by the Chemical Rubber Co., Cleveland, Ohio. The amount of osmopolymer comprising a hydro-activated layer can be from about 5% to 100%.

The expandable layer in another manufacture can comprise an osmotically effective compound that comprises inorganic and organic compounds that exhibit an osmotic pressure gradient across a semipermeable wall against an external fluid. The osmotically effective compounds, as with the osmopolymers, imbibe fluid into the osmotic system, thereby making available fluid to push against the inner wall, i.e., in some embodiments, the barrier layer and/or the wall of the soft or hard capsule for pushing active agent from the dosage form. The osmotically effective compounds are known also as osmotically effective solutes, and also as osmagents. Osmotically effective solutes that can be used comprise magnesium sulfate, magnesium chloride, potassium sulfate, sodium sulfate, lithium sulfate, potassium acid phosphate, mannitol, urea, inositol, magnesium succinate, tartaric acid, carbohydrates such as raffinose, sucrose, glucose, lactose, sorbitol, and mixtures therefor. The amount of osmagent in can be from about 5% to 100% of the weight of the layer. The expandable layer optionally comprises an osmopolymer and an osmagent with the total amount of osmopolymer and osmagent equal to 100%. Osmotically effective solutes are known to the prior art as described in U.S. Pat. No. 4,783,337.

Protective subcoat, inner wall 90, is permeable to the passage of gastric fluid entering the compartment defined by wall 20 and provides a protective function that reduces the degradation of paliperidone under stress conditions.

Inner wall 90 further provides a lubricating function that facilitates the movement of first drug layer 30, second drug layer 40 and push layer 50 toward exit 60. Inner wall 90 may be formed from hydrophilic materials and excipients. Outer wall 20 is semipermeable, allowing gastric fluid to enter the compartment, but preventing the passage of the materials comprising the core in the compartment. The deliverable drug formulation is released from exit 60 upon osmotic operation of the osmotic oral dosage form.

Inner wall 90 also reduces friction between the external surface of drug layer 30 and drug layer 40, and the inner surface of wall 20. Inner wall 90 promotes release of the drug composition from the compartment and reduces the amount of residual drug composition remaining in the compartment at the end of the delivery period, particularly when the slurry, suspension or solution of the drug composition that is being dispensed is highly viscous during the period of time in which it is being dispensed. In dosage forms with hydrophobic agents and no inner wall, it has been observed that significant residual amounts of drug may remain in the device after the period of delivery has been completed. In some instances, amounts of 20% or greater may remain in the dosage form at the end of a twenty-four hour period when tested in a release rate assay.

Inner wall 90 is formed as an inner coat of a flow-promoting agent, i.e., an agent that lowers the frictional force between the outer wall 20 and the external surface of drug layer 40. Inner wall 90 appears to reduce the frictional forces between outer wall 20 and the outer surface of drug layer 30 and drug layer 40, thus allowing for more complete delivery of drug from the device. Particularly in the case of active compounds having a high cost, such an improvement presents substantial economic advantages since it is not necessary to load the drug layer with an excess of drug to insure that the minimum amount of drug required will be delivered. Inner wall 90 may be formed as a coating applied over the compressed core.

Inner wall 90 is further characterized by a protective agent, i.e., an agent that reduces the degradation of paliperidone in drug layer 30 and drug layer 40. Particularly in the case of active compounds having a high cost, such an improvement presents substantial economic advantages. Inner wall 90 may be formed as a coating applied over the compressed core.

Inner wall 90 typically may be 0.01 to 5 mm thick, more typically 0.5 to 5 mm thick, and it comprises a member selected from hydrogels, gelatin, low molecular weight polyethylene oxides, e.g., less than 100,000 MW, hydroxyalkylcelluloses, e.g., hydroxyethylcellulose, hydroxypropylcellulose, hydroxyisopropylcelluose, hydroxybutylcellulose and hydroxyphenylcellulose, and hydroxyalkyl alkylcelluloses, e.g., hydroxypropyl methylcellulose, and mixtures thereof. The hydroxyalkylcelluloses comprise polymers having a 9,500 to 1,250,000 number-average molecular weight. For example, hydroxypropyl celluloses having number average molecular weights of 80,000 to 850,000 are useful. The inner wall may be prepared from conventional solutions or suspensions of the aforementioned materials in aqueous solvents or inert organic solvents.

Preferred materials for the inner wall include hydroxypropyl cellulose, hydroxyethyl cellulose, hydroxypropyl methyl cellulose, povidone [poly(vinylpyrrolidone)], polyethylene glycol, and mixtures thereof.

Most preferred are mixtures of hydroxypropyl cellulose and povidone, prepared in organic solvents, particularly organic polar solvents such as lower alkanols having 1-8 carbon atoms, preferably ethanol, mixtures of hydroxyethyl cellolose and hydroxypropyl methyl cellulose prepared in aqueous solution, and mixtures of hydroxyethyl cellulose and polyethylene glycol prepared in aqueous solution. Most preferably, the inner wall comprises a mixture of hydroxypropyl cellulose and providone prepared in ethanol.

It is preferred that inner wall 90 comprises between about 50% and about 90% hydroxypropylcellulose identified as EF having an average molecular weight of about 80,000 and between about 10% and about 50% polyvinylpyrrolidone identified as K29-32.

Conveniently, the weight of the inner wall applied to the compressed core may be correlated with the thickness of the inner wall and residual drug remaining in a dosage form in a release rate assay such as described herein. As such, during manufacturing operations, the thickness of the inner wall may be controlled by controlling the weight of the inner wall taken up in the coating operation.

When inner wall 90 is formed as a subcoat, i.e., by coating onto the tabletted composite including one or all of the first drug layer, second drug layer and push layer, the inner wall can fill in surface irregularities formed on the core by the tabletting process. The resulting smooth external surface facilitates slippage between the coated composite core and the semipermeable wall during dispensing of the drug, resulting in a lower amount of residual drug composition remaining in the device at the end of the dosing period. When inner wall 90 is fabricated of a gel-forming material, contact with water in the environment of use facilitates formation of the gel or gel-like inner coat having a viscosity that may promote and enhance slippage between outer wall 20 and drug layer 30 and drug layer 40.

Pan coating may be conveniently used to provide the completed dosage form, except for the exit orifice. In the pan coating system, the wall-forming composition for the inner wall or the outer wall, as the case may be, is deposited by successive spraying of the appropriate wall composition onto the compressed trilayered or multilayered core comprising the drug layers, optional barrier layer and push layer, accompanied by tumbling in a rotating pan. A pan coater is used because of its availability at commercial scale. Other techniques can be used for coating the compressed core. Once coated, the wall is dried in a forced-air oven or in a temperature and humidity controlled oven to free the dosage form of solvent(s) used in the manufacturing. Drying conditions will be conventionally chosen on the basis of available equipment, ambient conditions, solvents, coatings, coating thickness, and the like.

Other coating techniques can also be employed. For example, the wall or walls of the dosage form may be formed in one technique using the air-suspension procedure. This procedure consists of suspending and tumbling the compressed core in a current of air and the semipermeable wall forming composition, until the wall is applied to the core. The air-suspension procedure is well suited for independently forming the wall of the dosage form. The air-suspension procedure is described in U.S. Pat. No. 2,799,241; in J. Am. Pharm. Assoc., Vol. 48, pp. 451-459 (1959); and, ibid., Vol. 49, pp. 82-84 (1960). The dosage form also can be coated with a Wurster® air-suspension coater using, for example, methylene dichloride methanol as a cosolvent for the wall forming material. An AEROMATIC® air-suspension coater can be used employing a cosolvent.

In an embodiment, the sustained release dosage form of the invention is provided with at least one exit 60 as shown in FIG. 2. Exit 60 cooperates with the compressed core for the uniform release of drug from the dosage form. The exit can be provided during the manufacture of the dosage form or during drug delivery by the dosage form in a fluid environment of use.

One or more exit orifices are drilled in the drug layer end of the dosage form, and optional water soluble overcoats, which may be colored (e.g., OPADRY® colored coatings) or clear (e.g., OPADRY® Clear), may be coated on the dosage form to provide the finished dosage form.

Exit 60 may include an orifice that is formed or formable from a substance or polymer that erodes, dissolves or is leached from the outer wall to thereby form an exit orifice. The substance or polymer may include, for example, an erodible poly(glycolic) acid or poly(lactic) acid in the semipermeable wall; a gelatinous filament; a water-removable poly(vinyl alcohol); a leachable compound, such as a fluid removable pore-former selected from the group consisting of inorganic and organic salt, oxide and carbohydrate.

An exit, or a plurality of exits, can be formed by leaching a member selected from the group consisting of sorbitol, lactose, fructose, glucose, mannose, galactose, talose, sodium chloride, potassium chloride, sodium citrate and mannitol to provide a uniform-release dimensioned pore-exit orifice.

The exit can have any shape, such as round, triangular, square, elliptical and the like for the uniform metered dose release of a drug from the dosage form.

The sustained release dosage form can be constructed with one or more exits in spaced-apart relation or one or more surfaces of the sustained release dosage form.

Drilling, including mechanical and laser drilling, through the semipermeable wall can be used to form the exit orifice. Such exits and equipment for forming such exits are disclosed in U.S. Pat. No. 3,916,899, by Theeuwes and Higuchi and in U.S. Pat. No. 4,088,864, by Theeuwes, et al. It is presently preferred to utilize two exits of equal diameter. In a preferred embodiment, exit 60 penetrates through subcoat 90, if present, to drug layer 30.

Dosage forms in accordance with the embodiments depicted in FIG. 1 are manufactured by standard techniques. For example, the dosage form may be manufactured by the wet granulation technique. In the wet granulation technique, the drug and carrier are blended using an organic solvent, such as denatured anhydrous ethanol, as the granulation fluid. The remaining ingredients can be dissolved in a portion of the granulation fluid, such as the solvent described above, and this latter prepared wet blend is slowly added to the drug blend with continual mixing in the blender. The granulating fluid is added until a wet blend is produced, which wet mass blend is then forced through a predetermined screen onto oven trays. The blend is dried for 18 to 24 hours at 24° C. to 35° C. in a forced-air oven. The dried granules are then sized. Next, magnesium stearate, or another suitable lubricant, is added to the drug granulation, and the granulation is put into milling jars and mixed on a jar mill for 10 minutes. The composition is pressed into a layer, for example, in a Manesty® press or a Korsch LCT press. For a trilayered core, granules or powders of the drug layer compositions and push layer composition are sequentially placed in an appropriately-sized die with intermediate compression steps being applied to each of the first two layers, followed by a final compression step after the last layer is added to the die to form the trilayered core. The intermediate compression typically takes place under a force of about 50-100 newtons. Final stage compression typically takes place at a force of 3500 newtons or greater, often 3500-5000 newtons. The compressed cores are fed to a dry coater press, e.g., Kilian® Dry Coater press, and subsequently coated with the wall materials as described above.

In another embodiment, the drug and other ingredients comprising the drug layer are blended and pressed into a solid layer. The layer possesses dimensions that correspond to the internal dimensions of the area the layer is to occupy in the dosage form, and it also possesses dimensions corresponding to the push layer, if included, for forming a contacting arrangement therewith. The drug and other ingredients can also be blended with a solvent and mixed into a solid or semisolid form by conventional methods, such as ballmilling, calendering, stirring or rollmilling, and then pressed into a preselected shape. Next, if included, a layer of osmopolymer composition is placed in contact with the layer of drug in a like manner. The layering of the drug formulation and the osmopolymer layer can be fabricated by conventional two-layer press techniques. An analogous procedure may be followed for the preparation of the trilayered core. The compressed cores then may be coated with the inner wall material and the semipermeable wall material as described above.

Another manufacturing process that can be used comprises blending the powdered ingredients for each layer in a fluid bed granulator. After the powdered ingredients are dry blended in the granulator, a granulating fluid, for example, poly(vinylpyrrolidone) in water, is sprayed onto the powders. The coated powders are then dried in the granulator. This process granulates all the ingredients present therein while adding the granulating fluid. After the granules are dried, a lubricant, such as stearic acid or magnesium stearate, is mixed into the granulation using a blender e.g., V-blender or tote blender. The granules are then pressed in the manner described above.

Exemplary solvents suitable for manufacturing the dosage form components comprise aqueous or inert organic solvents that do not adversely harm the materials used in the system. The solvents broadly include members selected from the group consisting of aqueous solvents, alcohols, ketones, esters, ethers, aliphatic hydrocarbons, halogenated solvents, cycloaliphatics, aromatics, heterocyclic solvents and mixtures thereof. Typical solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride nitroethane, nitropropane tetrachloroethane, ethyl ether, isopropyl ether, cyclohexane, cyclooctane, benzene, toluene, naphtha, 1,4-dioxane, tetrahydrofuran, diglyme, water, aqueous solvents containing inorganic salts such as sodium chloride, calcium chloride, and the like, and mixtures thereof such as acetone and water, acetone and methanol, acetone and ethyl alcohol, methylene dichloride and methanol, and ethylene dichloride and methanol.

One important consideration in the practice of this invention is the physical state of the benzisoxazole derivative to be delivered by the dosage form. In certain embodiments, the benzisoxazole derivatives may be in a paste or liquid state. In such cases solid dosage forms may not be suitable for use in the practice of this invention. Instead, dosage forms capable of delivering substances in a paste or liquid state should be used.

The present invention provides a liquid formulation of substances for use with oral osmotic devices. Oral osmotic devices for delivering liquid formulations and methods of using them are known in the art, for example, as described and claimed in the following U.S. patents owned by ALZA corporation: U.S. Pat. Nos. 6,419,952; 6,174,547; 6,551,613; 5,324,280; 4,111,201; and 6,174,547. Methods of using oral osmotic devices for delivering therapeutic agents at an ascending rate of release can be found in International Application Numbers WO 98/06380, WO 98/23263, and WO 99/62496.

Exemplary liquid carriers for the present invention include lipophilic solvents (e.g., oils and lipids), surfactants, and hydrophilic solvents. Exemplary lipophilic solvents, for example, include, but are not limited to, Capmul PG-8, Caprol MPGO, Capryol 90, Plurol Oleique CC 497, Capmul MOM, Labrafac PG, N-Decyl Alcohol, Caprol 10G10O, Oleic Acid, Vitamin E, Maisine 35-1, Gelucire 33/01, Gelucire 44/14, Lauryl Alcohol, Captex 355EP, Captex 500, Capylic/Caplic Triglyceride, Peceol, Caprol ET, Labrafil M2125 CS, Labrafac CC, Labrafil M 1944 CS, Captex 8277, Myvacet 9-45, Isopropyl Nyristate, Caprol PGE 860, Olive Oil, Plurol Oleique, Peanut Oil, Captex 300 Low C6, and Capric Acid.

Exemplary surfactants for example, include, but are not limited to, Vitamin E TPGS, Cremophor (grades EL, EL-P, and RH40), Labrasol, Tween (grades 20, 60, 80), Pluronic (grades L-31, L-35, L-42, L-64, and L-121), Acconon S-35, Solutol HS-15, and Span (grades 20, and 80). Exemplary hydrophilic solvents for example, include, but are not limited to, Isosorbide Dimethyl Ether, Polyethylene Glycol (PEG grades 300, 400, 600, 3000, 4000, 6000, and 8000) and Propylene Glycol (PG).

The skilled practitioner will understand that any formulation comprising a sufficient dosage of benzisoxazole derivative solubilized in a liquid carrier suitable for administration to a subject and for use in an osmotic device can be used in the present invention. In one exemplary embodiment of the present invention, the liquid carrier is PG, Solutol, Cremophor EL, or a combination thereof.

The liquid formulation according to the present invention can also comprise, for example, additional excipients such as an antioxidant, permeation enhancer and the like. Antioxidants can be provided to slow or effectively stop the rate of any autoxidizable material present in the capsule. Representative antioxidants can comprise a member selected from the group of ascorbic acid; alpha tocopherol; ascorbyl palmitate; ascorbates; isoascorbates; butylated hydroxyanisole; butylated hydroxytoluene; nordihydroguiaretic acid; esters of garlic acid comprising at least 3 carbon atoms comprising a member selected from the group consisting of propyl gallate, octyl gallate, decyl gallate, decyl gallate; 6-ethoxy-2,2,4-trimethyl-1,2-dihydro-quinoline; N-acetyl-2,6-di-t-butyl-p-aminophenol; butyl tyrosine; 3-tertiarybutyl-4-hydroxyanisole; 2-tertiary-butyl-4-hydroxyanisole; 4-chloro-2,6-ditertiary butyl phenol; 2,6-ditertiary butyl p-methoxy phenol; 2,6-ditertiary butyl-p-cresol: polymeric antioxidants; trihydroxybutyro-phenone physiologically acceptable salts of ascorbic acid, erythorbic acid, and ascorbyl acetate; calcium ascorbate; sodium ascorbate; sodium bisulfite; and the like. The amount of antioxidant used for the present purposes, for example, can be about 0.001% to 25% of the total weight of the composition present in the lumen. Antioxidants are known to the prior art in U.S. Pat. Nos. 2,707,154; 3,573,936; 3,637,772; 4,038,434; 4,186,465 and 4,559,237, each of which is hereby incorporated by reference in its entirety for all purposes.

The inventive liquid formulation can comprise permeation enhancers that facilitate absorption of the drug in the environment of use. Such enhancers can, for example, open the so-called “tight junctions” in the gastrointestinal tract or modify the effect of cellular components, such a p-glycoprotein and the like. Suitable enhancers can include alkali metal salts of salicyclic acid, such as sodium salicylate, caprylic or capric acid, such as sodium caprylate or sodium caprate, and the like. Enhancers can include, for example, the bile salts, such as sodium deoxycholate. Various p-glycoprotein modulators are described in U.S. Pat. Nos. 5,112,817 and 5,643,909. Various other absorption enhancing compounds and materials are described in U.S. Pat. No. 5,824,638. Enhancers can be used either alone or as mixtures in combination with other enhancers.

In certain embodiments, the inventive substances are administered as a self-emulsifying formulation. Like the other liquid carriers, the surfactant functions to prevent aggregation, reduce interfacial tension between constituents, enhance the free-flow of constituents, and lessen the incidence of constituent retention in the dosage form. The emulsion formulation of this invention comprises a surfactant that imparts emulsification. Exemplary surfactants can also include, for example, in addition to the surfactants listed above, a member selected from the group consisting of polyoxyethylenated castor oil comprising ethylene oxide in the concentration of 9 to 15 moles, polyoxyethylenated sorbitan monopalmitate, mono and tristearate comprising 20 moles of ethylene oxide, polyoxyethylenated sorbitan monostearate comprising 4 moles of ethylene oxide, polyoxyethylenated sorbitan trioleate comprising 20 moles of ethylene oxide, polyoxyethylene lauryl ether, polyoxyethylenated stearic acid comprising 40 to 50 moles of ethylene oxide, polyoxyethylenated stearyl alcohol comprising 2 moles of ethylene oxide, and polyoxyethylenated oleyl alcohol comprising 2 moles of ethylene oxide. The surfactants may be available from Atlas Chemical Industries.

The drug emulsified formulations of the present invention can initially comprise an oil and a non-ionic surfactant. The oil phase of the emulsion comprises any pharmaceutically acceptable oil which is not immiscible with water. The oil can be an edible liquid such as a non-polar ester of an unsaturated fatty acid, derivatives of such esters, or mixtures of such esters. The oil can be vegetable, mineral, animal or marine in origin. Examples of non-toxic oils can also include, for example, in addition to the surfactants listed above, a member selected from the group consisting of peanut oil, cottonseed oil, sesame oil, corn oil, almond oil, mineral oil, castor oil, coconut oil, palm oil, cocoa butter, safflower, a mixture of mono- and diglycerides of 16 to 18 carbon atoms, unsaturated fatty acids, fractionated triglycerides derived from coconut oil, fractionated liquid triglycerides derived from short chain 10 to 15 carbon atoms fatty acids, acetylated monoglycerides, acetylated diglycerides, acetylated triglycerides, olein known also as glyceral trioleate, palmitin known as glyceryl tripalmitate, stearin known also as glyceryl tristearate, lauric acid hexylester, oleic acid oleylester, glycolyzed ethoxylated glycerides of natural oils, branched fatty acids with 13 molecules of ethyleneoxide, and oleic acid decylester. The concentration of oil, or oil derivative in the emulsion formulation can be from about 1 wt % to about 40 wt %, with the wt % of all constituents in the emulsion preparation equal to 100 wt %. The oils are disclosed in Pharmaceutical Sciences by Remington, 17th Ed., pp. 403-405, (1985) published by Mark Publishing Co., in Encyclopedia of Chemistry, by Van Nostrand Reinhold, 4th Ed., pp. 644-645, (1984) published by Van Nostrand Reinhold Co.; and in U.S. Pat. No. 4,259,323.

The amount of benzisoxazole derivative incorporated in the dosage forms of the present invention is generally from about 10% to about 90% by weight of the composition depending upon the therapeutic indication and the desired administration period, e.g., every 12 hours, every 24 hours, and the like. Depending on the dose of benzisoxazole derivative desired to be administered, one or more of the dosage forms can be administered.

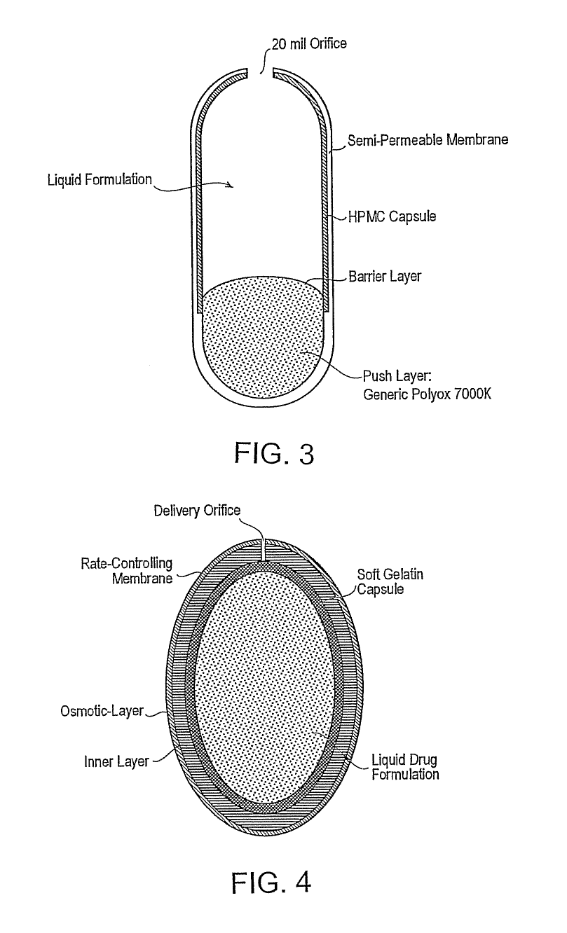

The osmotic dosage forms of the present invention can possess two distinct forms, a soft capsule form (shown in FIG. 4) and a hard capsule form (shown in FIG. 3). The soft capsule, as used by the present invention, preferably in its final form comprises one piece. The one-piece capsule is of a sealed construction encapsulating the drug formulation therein. The capsule can be made by various processes including the plate process, the rotary die process, the reciprocating die process, and the continuous process. An example of the plate process is as follows. The plate process uses a set of molds. A warm sheet of a prepared capsule lamina-forming material is laid over the lower mold and the formulation poured on it. A second sheet of the lamina-forming material is placed over the formulation followed by the top mold. The mold set is placed under a press and a pressure applied, with or without heat, to form a unit capsule. The capsules are washed with a solvent for removing excess agent formulation from the exterior of the capsule, and the air-dried capsule is encapsulated with a semipermeable wall. The rotary die process uses two continuous films of capsule lamina-forming material that are brought into convergence between a pair of revolving dies and an injector wedge. The process fills and seals the capsule in dual and coincident operations. In this process, the sheets of capsule lamina-forming material are fed over guide rolls, and then down between the wedge injector and the die rolls. The agent formulation to be encapsulated flows by gravity into a positive displacement pump. The pump meters the agent formulation through the wedge injector and into the sheets between the die rolls. The bottom of the wedge contains small orifices lined up with the die pockets of the die rolls. The capsule is about half-sealed when the pressure of pumped agent formulation forces the sheets into the die pockets, wherein the capsules are simultaneously filled, shaped, hermetically sealed and cut from the sheets of lamina-forming materials. The sealing of the capsule is achieved by mechanical pressure on the die rolls and by heating of the sheets of lamina-forming materials by the wedge. After manufacture, the agent formulation-filled capsules are dried in the presence of forced air, and a semipermeable lamina encapsulated thereto.

The reciprocating die process produces capsules by leading two films of capsule lamina-forming material between a set of vertical dies. The dies as they close, open, and close perform as a continuous vertical plate forming row after row of pockets across the film. The pockets are filled with an inventive formulation, and as the pockets move through the dies, they are sealed, shaped, and cut from the moving film as capsules filled with agent formulation. A semipermeable encapsulating lamina is coated thereon to yield the capsule. The continuous process is a manufacturing system that also uses rotary dies, with the added feature that the process can successfully fill active agent in dry powder form into a soft capsule, in addition to encapsulating liquids. The filled capsule of the continuous process is encapsulated with a semipermeable polymeric material to yield the capsule. Procedures for manufacturing soft capsules are disclosed in U.S. Pat. No. 4,627,850 and U.S. Pat. No. 6,419,952.

The dosage forms of the present invention can also be made from an injection-moldable composition by an injection-molding technique. Injection-moldable compositions provided for injection-molding into the semipermeable wall comprise a thermoplastic polymer, or the compositions comprise a mixture of thermoplastic polymers and optional injection-molding ingredients. The thermoplastic polymer that can be used for the present purpose comprise polymers that have a low softening point, for example, below 200° C., preferably within the range of 40° C. to 180° C. The polymers, are preferably synthetic resins, addition polymerized resins, such as polyamides, resins obtained from diepoxides and primary alkanolamines, resins of glycerine and phthalic anhydrides, polymethane, polyvinyl resins, polymer resins with end-positions free or esterified carboxyl or caboxamide groups, for example with acrylic acid, acrylic amide, or acrylic acid esters, polycaprolactone, and its copolymers with dilactide, diglycolide, valerolactone and decalactone, a resin composition comprising polycaprolactone and polyalkylene oxide, and a resin composition comprising polycaprolactone, a polyalkylene oxide such as polyethylene oxide, poly(cellulose) such as poly(hydroxypropylmethylcellulose), poly(hydroxyethylmethylcellulose), and poly(hydroxypropylcellulose). The membrane forming composition can comprise optional membrane-forming ingredients such as polyethylene glycol, talcum, polyvinylalcohol, lactose, or polyvinyl pyrrolidone. The compositions for forming an injection-molding polymer composition can comprise 100% thermoplastic polymer. The composition in another embodiment comprises 10% to 99% of a thermoplastic polymer and 1% to 90% of a different polymer with the total equal to 100%. The invention provides also a thermoplastic polymer composition comprising 1% to 98% of a first thermoplastic polymer, 1% to 90% of a different, second polymer and 1% to 90% of a different, third polymer with all polymers equal to 100%. Representation composition comprises 20% to 90% of thermoplastic polycaprolactone and 10% to 80% of poly(alkylene oxide); a composition comprising 20% to 90% polycaprolactone and 10% to 60% of poly(ethylene oxide) with the ingredients equal to 100%; a composition comprising 10% to 97% of polycaprolactone, 10% to 97% poly(alkylene oxide), and 1% to 97% of poly(ethylene glycol) with all ingredients equal to 100%; a composition comprising 20% to 90% polycaprolactone and 10% to 80% of poly(hydroxypropylcellulose) with all ingredients equal to 100%; and a composition comprising 1% to 90% polycaprolactone, 1% to 90% poly(ethylene oxide), 1% to 90% poly(hydroxypropylcellulose) and 1% to 90% poly(ethylene glycol) with all ingredients equal to 100%. The percent expressed is weight percent wt %.

In another embodiment of the invention, a composition for injection-molding to provide a membrane can be prepared by blending a composition comprising a polycaprolactone 63 wt %, polyethylene oxide 27 wt %, and polyethylene glycol 10 wt % in a conventional mixing machine, such as a Moriyama™ Mixer at 65° C. to 95° C., with the ingredients added to the mixer in the following addition sequence, polycaprolactone, polyethylene oxide and polyethylene glycol. In one example, all the ingredients are mixed for 135 minutes at a rotor speed of 10 to 20 rpm. Next, the blend is fed to a Baker Perkins Kneader™ extruder at 80° C. to 90° C., at a pump speed of 10 rpm and a screw speed of 22 rpm, and then cooled to 10° C. to 12° C., to reach a uniform temperature. Then, the cooled extruded composition is fed to an Albe Pelletizer, converted into pellets at 250° C., and a length of 5 mm. The pellets next are fed into an injection-molding machine, an Arburg Allrounder™ at 200° F. to 350° C. (93° C. to 177° C.), heated to a molten polymeric composition, and the liquid polymer composition forced into a mold cavity at high pressure and speed until the mold is filled and the composition comprising the polymers are solidified into a preselected shape. The parameters for the injection-molding consists of a band temperature through zone 1 to zone 5 of the barrel of 195° F. (91° C.) to 375° F., (191° C.), an injection-molding pressure of 1818 bar, a speed of 55 cm3/s, and a mold temperature of 75° C. The injection-molding compositions and injection-molding procedures are disclosed in U.S. Pat. No. 5,614,578.

Alternatively, the capsule can be made conveniently in two parts, with one part (the “cap”) slipping over and capping the other part (the “body”) as long as the capsule is deformable under the forces exerted by the expandable layer and seals to prevent leakage of the liquid, active agent formulation from between the telescoping portions of the body and cap. The two parts completely surround and capsulate the internal lumen that contains the liquid, active agent formulation, which can contain useful additives. The two parts can be fitted together after the body is filled with a preselected formulation. The assembly can be done by slipping or telescoping the cap section over the body section, and sealing the cap and body, thereby completely surrounding and encapsulating the formulation of active agent.

Soft capsules typically have a wall thickness that is greater than the wall thickness of hard capsules. For example, soft capsules can, for example, have a wall thickness on the order of 10-40 mils, about 20 mils being typical, whereas hard capsules can, for example, have a wall thickness on the order of 2-6 mils, about 4 mils being typical.

In one embodiment of the dosage system, a soft capsule can be of single unit construction and can be surrounded by an unsymmetrical hydro-activated layer as the expandable layer. The expandable layer will generally be unsymmetrical and have a thicker portion remote from the exit orifice. As the hydro-activated layer imbibes and/or absorbs external fluid, it expands and applies a push pressure against the wall of capsule and optional barrier layer and forces active agent formulation through the exit orifice. The presence of an unsymmetrical layer functions to assure that the maximum dose of agent is delivered from the dosage form, as the thicker section of layer distant from passageway swells and moves towards the orifice.

In yet another configuration, the expandable layer can be formed in discrete sections that do not entirely encompass an optionally barrier layer-coated capsule. The expandable layer can be a single element that is formed to fit the shape of the capsule at the area of contact. The expandable layer can be fabricated conveniently by tableting to form the concave surface that is complementary to the external surface of the barrier-coated capsule. Appropriate tooling such as a convex punch in a conventional tableting press can provide the necessary complementary shape for the expandable layer. In this case, the expandable layer is granulated and compressed, rather than formed as a coating. The methods of formation of an expandable layer by tableting are well known, having been described, for example in U.S. Pat. Nos. 4,915,949; 5,126,142; 5,660,861; 5,633,011; 5,190,765; 5,252,338; 5,620,705; 4,931,285; 5,006,346; 5,024,842; and 5,160,743.

In some embodiments, a barrier layer can be first coated onto the capsule and then the tableted, expandable layer is attached to the barrier-coated capsule with a biologically compatible adhesive. Suitable adhesives include, for example, starch paste, aqueous gelatin solution, aqueous gelatin/glycerin solution, acrylate-vinylacetate based adhesives such as Duro-Tak adhesives (National Starch and Chemical Company), aqueous solutions of water soluble hydrophilic polymers such as hydroxypropyl methyl cellulose, hydroxymethyl cellulose, hydroxyethyl cellulose, and the like. That intermediate dosage form can be then coated with a semipermeable layer. The exit orifice is formed in the side or end of the capsule opposite the expandable layer section. As the expandable layer imbibes fluid, it will swell. Since it is constrained by the semipermeable layer, as it expands it will compress the barrier-coated capsule and express the liquid, active agent formulation from the interior of the capsule into the environment of use.

The hard capsules are typically composed of two parts, a cap and a body, which are fitted together after the larger body is filled with a preselected appropriate formulation. This can be done by slipping or telescoping the cap section over the body section, thus completely surrounding and encapsulating the useful agent formulation. Hard capsules can be made, for example, by dipping stainless steel molds into a bath containing a solution of a capsule lamina-forming material to coat the mold with the material. Then, the molds are withdrawn, cooled, and dried in a current of air. The capsule is stripped from the mold and trimmed to yield a lamina member with an internal lumen. The engaging cap that telescopically caps the formulation receiving body is made in a similar manner. Then, the closed and filled capsule can be encapsulated with a semipermeable lamina. The semipermeable lamina can be applied to capsule parts before or after parts and are joined into the final capsule. In another embodiment, the hard capsules can be made with each part having matched locking rings near their opened end that permit joining and locking together the overlapping cap and body after filling with formulation. In this embodiment, a pair of matched locking rings are formed into the cap portion and the body portion, and these rings provide the locking means for securely holding together the capsule. The capsule can be manually filled with the formulation, or they can be machine filled with the formulation. In the final manufacture, the hard capsule is encapsulated with a semipermeable lamina permeable to the passage of fluid and substantially impermeable to the passage of useful agent. Methods of forming hard cap dosage forms are described in U.S. Pat. No. 6,174,547, U.S. Pat. Nos. 6,596,314, 6,419,952, and 6,174,547.

The hard and soft capsules can comprise, for example, gelatin; gelatin having a viscosity of 15 to 30 millipoises and a bloom strength up to 150 grams; gelatin having a bloom value of 160 to 250; a composition comprising gelatin, glycerine, water and titanium dioxide; a composition comprising gelatin, erythrosin, iron oxide and titanium dioxide; a composition comprising gelatin, glycerine, sorbitol, potassium sorbate and titanium dioxide; a composition comprising gelatin, acacia glycerine, and water; and the like. Materials useful for forming capsule wall are known in U.S. Pat. No. 4,627,850; and in U.S. Pat. No. 4,663,148. Alternatively, the capsules can be made out of materials other than gelatin (see for example, products made by BioProgres plc).

The capsules typically can be provided, for example, in sizes from about 3 to about 22 minims (1 minim being equal to 0.0616 ml) and in shapes of oval, oblong or others. They can be provided in standard shape and various standard sizes, conventionally designated as (000), (00), (0), (1), (2), (3), (4), and (5). The largest number corresponds to the smallest size. Non-standard shapes can be used as well. In either case of soft capsule or hard capsule, non-conventional shapes and sizes can be provided if required for a particular application.

The osmotic devices of the present invention may comprise a semipermeable wall permeable to the passage of exterior biological fluid and substantially impermeable to the passage of benzisoxazole derivative formulation. The selectively permeable compositions used for forming the wall are essentially non-erodible and they are insoluble in biological fluids during the life of the osmotic system. The semipermeable wall comprises a composition that does not adversely affect the host, the benzisoxazole derivative formulation, an osmopolymer, osmagent and the like. Materials useful in the formation of a semipermeable wall are disclosed elsewhere herein.

The semipermeable wall can also comprise a flux regulating agent. Materials useful flux regulating agents are disclosed elsewhere herein. Other materials that can be used to form the semipermeable wall for imparting flexibility and elongation properties to the semipermeable wall are also disclosed elsewhere herein.

The semipermeable wall surrounds and forms a compartment containing a one or a plurality of layers, one of which is an expandable layer which in some embodiments, can contain osmotic agents. The composition of such expandable layers is disclosed elsewhere herein.

In certain solid and liquid embodiments, the dosage forms further can comprise a barrier layer. The barrier layer in certain embodiments is deformable under the pressure exerted by the expandable layer and will be impermeable (or less permeable) to fluids and materials that can be present in the expandable layer, the liquid active agent formulation and in the environment of use, during delivery of the active agent formulation. A certain degree of permeability of the barrier layer can be permitted if the delivery rate of the active agent formulation is not detrimentally effected. However, it is preferred that barrier layer not completely transport through it fluids and materials in the dosage form and the environment of use during the period of delivery of the active agent. The barrier layer can be deformable under forces applied by expandable layer so as to permit compression of capsule to force the liquid, active agent formulation from the exit orifice. In some embodiments, the barrier layer will be deformable to such an extent that it create a seal between the expandable layer and the semipermeable layer in the area where the exit orifice is formed. In that manner, the barrier layer will deform or flow to a limited extent to seal the initially, exposed areas of the expandable layer and the semipermeable layer when the exit orifice is being formed, such as by drilling or the like, or during the initial stages of operation. When sealed, the only avenue for liquid permeation into the expandable layer is through the semipermeable layer, and there is no back-flow of fluid into the expandable layer through the exit orifice.

Suitable materials for forming the barrier layer can include, for example, polyethylene, polystyrene, ethylene-vinyl acetate copolymers, polycaprolactone and Hytrel™ polyester elastomers (Du Pont), cellulose acetate, cellulose acetate pseudolatex (such as described in U.S. Pat. No. 5,024,842), cellulose acetate propionate, cellulose acetate butyrate, ethyl cellulose, ethyl cellulose pseudolatex (such as Surelease™ as supplied by 10 Colorcon, West Point, Pa. or Aquacoat™ as supplied by FMC Corporation, Philadelphia, Pa.), nitrocellulose, polylactic acid, poly-glycolic acid, polylactide glycolide copolymers, collagen, polyvinyl alcohol, polyvinyl acetate, polyethylene vinylacetate, polyethylene teraphthalate, polybutadiene styrene, polyisobutylene, polyisobutylene isoprene copolymer, polyvinyl chloride, polyvinylidene chloride-vinyl chloride copolymer, copolymers of acrylic acid and methacrylic acid esters, copolymers of methylmethacrylate and ethylacrylate, latex of acrylate esters (such as EUDRAGIT® supplied by RohmPharma, Darmstaat, Germany), polypropylene, copolymers of propylene oxide and ethylene oxide, propylene oxide ethylene oxide block copolymers, ethylenevinyl alcohol copolymer, polysulfone, ethylene vinylalcohol copolymer, polyxylylenes, polyalkoxysilanes, polydimethyl siloxane, polyethylene glycol-silicone elastomers, electromagnetic irradiation crosslinked acrylics, silicones, or polyesters, thermally crosslinked acrylics, silicones, or polyesters, butadiene-styrene rubber, and blends of the above.

Preferred materials can include cellulose acetate, copolymers of acrylic acid and methacrylic acid esters, copolymers of methylmethacrylate and ethylacrylate, and latex of acrylate esters. Preferred copolymers can include poly(butyl methacrylate), (2-dimethylaminoethyl)methacrylate, methyl methacrylate) 1:2:1, 150,000, sold under the trademark EUDRAGIT® E; poly(ethyl acrylate, methyl methacrylate) 2:1, 800,000, sold under the trademark EUDRAGIT® NE 30 D; poly(methacrylic acid, methyl methacrylate) 1:1, 135,000, sold under the trademark EUDRAGIT® L; poly(methacrylic acid, ethyl acrylate) 1:1, 250,000, sold under the trademark EUDRAGIT® L; poly (methacrylic acid, methyl methacrylate) 1:2, 135,000, sold under the trademark EUDRAGIT S; poly(ethyl acrylate, methyl methacrylate, trimethylammonioethyl methacrylate chloride) 1:2:0.2, 150,000, sold under the trademark EUDRAGIT RL; poly (ethyl acrylate, methyl methacrylate, trimethylammonioethyl methacrylate chloride) 1:2:0.1, 150,000, sold as EUDRAGIT® RS. In each case, the ratio x:y:z indicates the molar proportions of the monomer units and the last number is the number average molecular weight of the polymer. Especially preferred are cellulose acetate containing plasticizers such as acetyl tributyl citrate and ethylacrylate methylmethylacrylate copolymers such as EUDRAGIT® NE.

The foregoing materials for use as the barrier layer can be formulated with plasticizers to make the barrier layer suitably deformable such that the force exerted by the expandable layer will collapse the compartment formed by the barrier layer to dispense the liquid, active agent formulation. Examples of typical plasticizers are as follows: polyhydric alcohols, triacetin, polyethylene glycol, glycerol, propylene glycol, acetate esters, glycerol triacetate, triethyl citrate, acetyl triethyl citrate, glycerides, acetylated monoglycerides, oils, mineral oil, castor oil and the like. The plasticizers can be blended into the material in amounts of 10-50 weight percent based on the weight of the material.

The various layers forming the barrier layer, expandable layer and semipermeable layer can be applied by conventional coating methods such as described in U.S. Pat. No. 5,324,280. While the barrier layer, expandable layer and semipermeable wall have been illustrated and described for convenience as single layers, each of those layers can be composites of several layers. For example, for particular applications it may be desirable to coat the capsule with a first layer of material that facilitates coating of a second layer having the permeability characteristics of the barrier layer. In that instance, the first and second layers comprise the barrier layer. Similar considerations would apply to the semipermeable layer and the expandable layer.

The exit orifice can be formed by mechanical drilling, laser drilling, eroding an erodible element, extracting, dissolving, bursting, or leaching a passageway former from the composite wall. The exit orifice can be a pore formed by leaching sorbitol, lactose or the like from a wall or layer as disclosed in U.S. Pat. No. 4,200,098. This patent discloses pores of controlled-size porosity formed by dissolving, extracting, or leaching a material from a wall, such as sorbitol from cellulose acetate. A preferred form of laser drilling is the use of a pulsed laser that incrementally removes material from the composite wall to the desired depth to form the exit orifice.

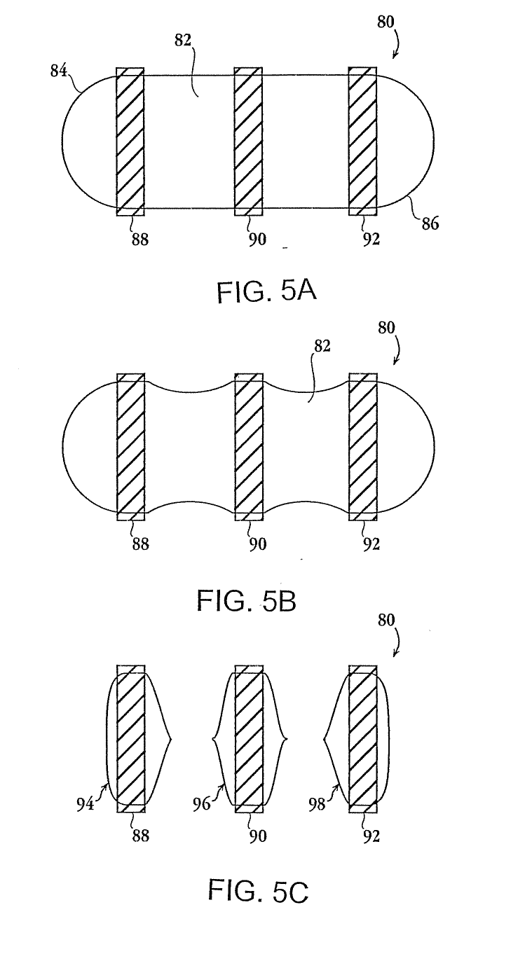

FIGS. 5A-5C illustrate another exemplary dosage form, known in the art and described in U.S. Pat. Nos. 5,534,263; 5,667,804; and 6,020,000. Briefly, a cross-sectional view of a dosage form 80 is shown prior to ingestion into the gastrointestinal tract in FIG. 5A. The dosage form is comprised of a cylindrically shaped matrix 82 comprising an inventive substance. Ends 84, 86 of matrix 82 are preferably rounded and convex in shape in order to ensure ease of ingestion. Bands 88, 90, and 92 concentrically surround the cylindrical matrix and are formed of a material that is relatively insoluble in an aqueous environment. Suitable materials are set forth in the patents noted above and elsewhere herein.

After ingestion of dosage form 80, regions of matrix 82 between bands 88, 90, 92 begin to erode, as illustrated in FIG. 5B. Erosion of the matrix initiates release of the inventive substance into the fluidic environment of the G.I. tract. As the dosage form continues transit through the G.I. tract, the matrix continues to erode, as illustrated in FIG. 5C. Here, erosion of the matrix has progressed to such an extent that the dosage form breaks into three pieces, 94, 96, 98. Erosion will continue until the matrix portions of each of the pieces have completely eroded. Bands 94, 96, 98 will thereafter be expelled from the G.I. tract.

In an embodiment, the inventive sustained release dosage forms comprise gastric retention dosage forms. U.S. Pat. No. 5,007,790 to Shell, granted Apr. 16, 1991 and entitled “Sustained-release oral drug dosage form” (“Shell”) discloses a gastric retention dosage form useful in the practice of this invention. Shell discloses sustained-release oral drug dosage forms that release drug in solution at a rate controlled by the solubility of the drug. The dosage form comprises a tablet or capsule which comprises a plurality of particles of a dispersion of a limited solubility drug in a hydrophilic, water-swellable, crosslinked polymer that maintains its physical integrity over the dosing lifetime but thereafter rapidly dissolves. Once ingested, the particles swell to promote gastric retention and permit the gastric fluid to penetrate the particles, dissolve drug and leach it from the particles. A benzisoxazole derivative may be incorporated into such a gastric retention dosage form, or others known in the art, in the practice of this invention.

Other approaches to achieving sustained release of drugs from oral dosage forms are known in the art. For example, diffusion systems such as reservoir devices and matrix devices, dissolution systems such as encapsulated dissolution systems (including, for example, “tiny time pills”) and matrix dissolution systems, combination diffusion/dissolution systems and ion-exchange resin systems are known and are disclosed in Remington's Pharmaceutical Sciences, 1990 ed., pp. 1682-1685. we need to also introduce any type of stomach platform that are designed to release drug in the upper gastrointestinal tract. Dosage forms that operate in accord with these other approaches are encompassed by the scope of the disclosure herein to the extent that the drug release characteristics and/or the blood plasma concentration characteristics as recited herein and in the claims describe those dosage forms either literally or equivalently.

U.S. Pat. Nos. 5,871,778 and 5,656,299 disclose sustained microsphere formulations having almost zero order rate of release of active component when administered to a patient. U.S. Pat. Nos. 5,654,008; 5,650,173; 5,770,231; 6,077,843; 6,368,632; and 5,965,168 disclose sustained-release microparticle compositions and their use for controlled delivery of active agents.

It will be appreciated the dosage forms described herein, particularly in FIGS. 1-5 are merely exemplary of a variety of dosage forms designed for and capable of achieving administration of the inventive substance(s). Those of skill in the pharmaceutical arts can identify other dosage forms that would be suitable. In particular, notice may be taken of United States Published Patent Application No. 20040092534, by Yam, et al., which is specifically incorporated by reference under the general statement of incorporation by reference in Section II above.

### V. Methods of Use

The inventive methods, compositions, and dosage forms are useful in treating a variety of indications that are treatable using benzisoxazole derivatives. In an aspect, the invention provides a method for treating an indication, such as a disease or disorder, in a patient by administering an inventive composition or dosage form that comprises one or more benzisoxazole derivatives. In an embodiment, a composition or dosage form comprising one or more benzisoxazole derivatives is administered to the patient via oral administration. The dose administered is generally adjusted in accord with the age, weight, and condition of the patient, taking into consideration the dosage form and the desired result. Inventive dosage forms may comprise one or more benzisoxazole derivatives, or pharmacologically active metabolites in combination.

While there has been described and pointed out features and advantages of the invention, as applied to present embodiments, those skilled in the medical art will appreciate that various modifications, changes, additions, and omissions in the method described in the specification can be made without departing from the spirit of the invention.

### VI. Examples

### Example 1

### Study to Characterize the Absorption of Risperidone Administered Colonically and Orally in Healthy Volunteers

The study investigated the absorption of risperidone administered colonically and orally in healthy volunteers. The objective of the study was to characterize colonic absorption of risperidone by comparing the AUC_{inf} values of risperidone, paliperidone (a risperidone metabolite) and the active moiety for the colonic treatments and the oral treatment. This was a single-center, two-sequence, open-label, three-treatment, three-period, randomized, crossover pilot study in healthy males. Twelve subjects were dosed with risperidone to ensure that at least 9 subjects completed all three treatments.

Each subject was to receive the following three treatments:
Treatment A—2 mg risperidone (50 ml of 0.04 mg/mL solution in water for injection) infused over 6 hours in the transverse colon
Treatment B—2 mg risperidone (50 ml of 0.04 mg/mL solution in water for injection) administered as a bolus (administered over ˜10 minutes) in the transverse colon
Treatment C—2 mg risperidone (50 ml of 0.04 mg/mL solution in water for injection) administered orally as a bolus

Subjects received the two colonic treatments in the first two periods; the oral treatment was planned to be the last treatment (Period 3). The nasoenteral tube was removed after dosing in each of the two colonic treatments. If in either of the colonic treatments the nasoenteral tube did not reach the colon, the tube was to be removed and the subject was to complete the oral treatment if he had not already received it. A colonic treatment could be attempted again in Period 3 if needed.

If after 6 days, the nasoenteral tube in either of the colonic treatments reached only the ascending colon, drug solution was to be administered into the ascending colon. If the subject received drug solution in the ascending colon during the first colonic treatment, attempts were to be made to administer the drug solution into the ascending colon during the second colonic treatment. The washout period between each treatment was minimum of 6 days and not more than 14 days. The washout period began at the end of dosing. Twenty blood samples were collected from each subject for measurement of risperidone plasma concentrations during each treatment session. Samples were obtained at 0 (pre-dose), 0.25, 0.5, 0.75, 1, 1.5, 2, 3, 4, 5, 6, 7, 8, 12, 24, 30, 36, 48, 54, and 60 hours after dosing.

Pharmacokinetic parameters such as AUCt, AUCinf, Cmax, Tmax, and t½ were calculated for risperidone and paliperidone and for the active moieties (i.e. sum of the two analytes risperidone+paliperidone) for each treatment and subject. RelativeBioavailability was estimated for the colonic treatments. A summary of the observed values of these parameters is provided in Table 1.

The bioavailability of risperidone following the 6-hour colonic infusion and the 10-minute colonic bolus relative to oral dosing was 75% and 63%, respectively. Relative bioavailability compared to oral dosing was estimated as follows:

The bioavailability of paliperidone following the 6-hour colonic infusion and the 10-minute colonic bolus relative to oral dosing was 55% and 51%, respectively. The bioavailability of active moiety (sum of risperidone and its metabolite, paliperidone) following the 6-hour risperidone colonic infusion and the 10-minute colonic bolus relative to oral dosing was 60% and 53%, respectively.

Mean drug-to-metabolite ratio of the AUC_{inf} values were similar in all three treatments, suggesting drug metabolism is similar following oral and colonic delivery (0.26, 0.33, and 0.31, for the oral solution, colonic infusion over 6 hours, and colonic bolus over 10 minutes, respectively).

TABLE 1
Mean Pharmacokinetic Parameter Values

| | Treatment A | | Treatment B | | | |
|---|---|---|---|---|---|---|
| | Colonic | | Colonic bolus | | Treatment C | |
| | infusion | | 2 mg | | 2 mg Oral | |
| | n | | | | | |
| Parameters | 11 | | 11 | | 12 | |
| | Mean (SD) Risperidone Pharmacokinetics | | | | | |
| Cₘₐₓ (ng/mL) | 4.93 | (4.26) | 7.31 | (5.04) | 16.24 | (7.60) |
| Tₘₐₓ (h) | 6.18 | (0.87) | 0.77 | (0.21) | 0.69 | (0.16) |
| t_{1/2} (h) | 4.11 | (4.01)³ | 3.32 | (2.83) | 3.31 | (1.56) |
| AUCₜ | 53.1 | (73.2) | 29.7 | (19.8) | 58.7 | (29.8) |
| (ng · h/mL) | | | | | | |
| AUC_{inf} | 56.1 | (78.0) | 30.6 | (20.1) | 59.7 | (30.0) |
| (ng · h/mL) | | | | | | |
| Relative | 75.0% | (74.0) | 63.4% | (44.8) | NA | (reference |
| | Mean (SD) Paliperidone Pharmacokinetics | | | | | |
| Cₘₐₓ (ng/mL) | 4.53 | (2.96) | 5.33 | (3.77) | 9.49 | (5.09) |
| Tₘₐₓ (h) | 10.83 | (8.56) | 3.91 | (1.76) | 5.50 | (2.68) |
| t_{1/2} (h) | 19.8 | (8.6)³ | 16.9 | (4.6) | 22.4 | (9.9) |
| AUCₜ | 113.7 | (67.9) | 109.7 | (70.2) | 211.7 | (79.6) |
| (ng · h/mL) | | | | | | |
| AUC_{inf} | 134.7 | (83.7) | 119.0 | (73.8) | 243.5 | (89.0) |
| (ng · h/mL) | | | | | | |
| Relative | 54.6% | (29.0) | 50.8% | (28.0) | NA | (reference |
| | Mean (SD) Active Moiety | | | | | |
| Cₘₐₓ (ng/mL)¹ | 9.10 | (5.93) | 11.24 | (7.02) | 23.42 | (9.66) |
| Tₘₐₓ (h)¹ | 7.01 | (0.04) | 0.96 | (0.33) | 0.78 | (0.20) |
| AUCₜ | 166.8 | (117.4) | 139.4 | (84.3) | 270.4 | (93.2) |
| (ng · h/mL)² | | | | | | |
| AUC_{inf} | 190.8 | (144.0) | 149.6 | (88.0) | 303.1 | (104.2) |
| (ng · h/mL)² | | | | | | |
| Relative | 60.0% | (40.4) | 52.5% | (29.5) | NA | (reference |
| | Mean (SD) Drug AUC_{inf}/Metabolite AUC_{inf} Ratio | | | | | |
| Risperidone | 0.33 | (0.33) | 0.31 | (0.26) | 0.26 | (0.13) |
| AUC_{inf}/ | | | | | | |
| 9-hydroxy- | | | | | | |
| risperidone | | | | | | |

¹Cₘₐₓ and Tₘₐₓ values estimated from the concentration profile of the sum of risperidone and paliperidone
²AUC values estimated by sum of AUC values of risperidone and paliperidone
³n = 10 for risperidone and n = 9 for paliperidone

### Example 2

### A Pharmacokinetic-Pharmacodynamic Study to Evaluate Various Dosing Regimens of Risperidone

This study was conducted to evaluate pharmacodynamic effects (postural changes in blood pressure and measurements of prolactin serum concentration) following three different dosing profiles of risperidone. Twenty-four of twenty-eight healthy volunteers completed the study. Eighteen subjects had pharmacokinetic and prolactin data in all three active treatments.

This study utilized three oral dosing schedules of risperidone and a placebo.
1. Ascend Profile (total dose of 5.0 mg risperidone as tablets)—total of 3.0 mg in divided doses over 10 hours on day 1 followed by a single 2 mg tablet on day 2.
2. Flat profile (total dose of 4.5 mg risperidone as tablets)—total of 2.5 mg in divided doses over 20 hours on day 1, followed by a single 2 mg tablet day 2.
3. IR profile (total dose of 4 mg risperidone)—2 mg tablets on 2 consecutive days.
4. Placebo solution administered at each dosing time

Table 2, below provides the dosing schedule for each system. All subjects received all four treatments randomly. There was a 6-to-10-day washout period between treatments. The washout period began after the last dose in each treatment period.

TABLE 2
Study medication Schedule (mg)

Treatment

| Dose | Dose | A | B | C | D |
|---|---|---|---|---|---|
| Number | Hour | Ascend | Flat | IR | Placebo |
| 1 | 0 | 0.5 | 1.0 | 2.0 | 0.0 |
| 2 | 1.75 | 0.25 | 0.0 | 0.0 | 0.0 |
| 3 | 3 | 0.25 | 0.25 | 0.0 | 0.0 |
| 4 | 4 | 0.25 | 0.0 | 0.0 | 0.0 |
| 5 | 5 | 0.25 | 0.0 | 0.0 | 0.0 |
| 6 | 6 | 0.25 | 0.25 | 0.0 | 0.0 |
| 7 | 7 | 0.25 | 0.0 | 0.0 | 0.0 |
| 8 | 7.75 | 0.25 | 0.0 | 0.0 | 0.0 |
| 9 | 8.5 | 0.25 | 0.0 | 0.0 | 0.0 |
| 10 | 9 | 0.0 | 0.25 | 0.0 | 0.0 |
| 11 | 9.25 | 0.25 | 0.0 | 0.0 | 0.0 |
| 12 | 10 | 0.25 | 0.0 | 0.0 | 0.0 |
| 13 | 12.5 | 0.0 | 0.25 | 0.0 | 0.0 |
| 14 | 16 | 0.0 | 0.25 | 0.0 | 0.0 |
| 15 | 20 | 0.0 | 0.25 | 0.0 | 0.0 |
| 16 | 24 | 2.0 | 2.0 | 2.0 | 0.0 |

| | | | | |
|---|---|---|---|---|
| Total Dose | 5.0 | 4.5 | 4.0 | 0.0 |
| (mg) | | | | |

Blood samples were collected from each subject during each treatment period for measurement of concentrations of risperidone and its metabolite paliperidone at the following times: 0 (predose), 0.5, 1.0, 2.0, 4.0, 7.0, 11.0, 12.5, 14.0, 18.0, 24.0, 24.5, 25.0, 26.0, 28.0, 32.0, 36.0 and 48.0 hours (h) post treatment initiation.

Blood samples for prolactin concentration were collected as described above for the pharmacokinetic blood samples. Postural changes in blood pressure and heart rate were assessed during each treatment period at the following times: 0 (predose), 20 minutes (min), 50 min, 1.0 h 50 min, 3 h 50 min, 6 h 50 min, 8 h 50 min, 10 h 50 min, 12 h 20 min, 13 h 50 min, 23 h 50 min, 24 h 20 min, 24 h 50 min, 25 h 50 min, 27 h 50 min, 31 h 50 min, 35 h 50 min, and 47 h 50 min post treatment initiation. An automated blood pressure monitor was used to collect blood pressure while supine and 2 and 3 minutes after standing up. Immediately after the standing blood pressure was measured subjects were asked the following questions:

Since standing up have you felt:

Dizzy? (rate on a 5 point scale from none to severe)

Faint? (rate on a 5 point scale from none to severe)

Pharmacokinetic parameters such as AUCt, AUCinf, Cmax, Tmax, and t½ were calculated for risperidone and paliperidone and for the active moiety (i.e. sum of the two analytes risperidone+paliperidone) for each treatment and subject for each day.

AUCt, Cmax, and Tmax were calculated for prolactin for each treatment and subject for each day.

The percentage of subjects with >20 mm Hg drop in systolic blood pressure (SBP) at 3 minutes of standing or with symptoms of orthostatic hypotension (dizziness or faintness) was summarized for Days 1 and 2.

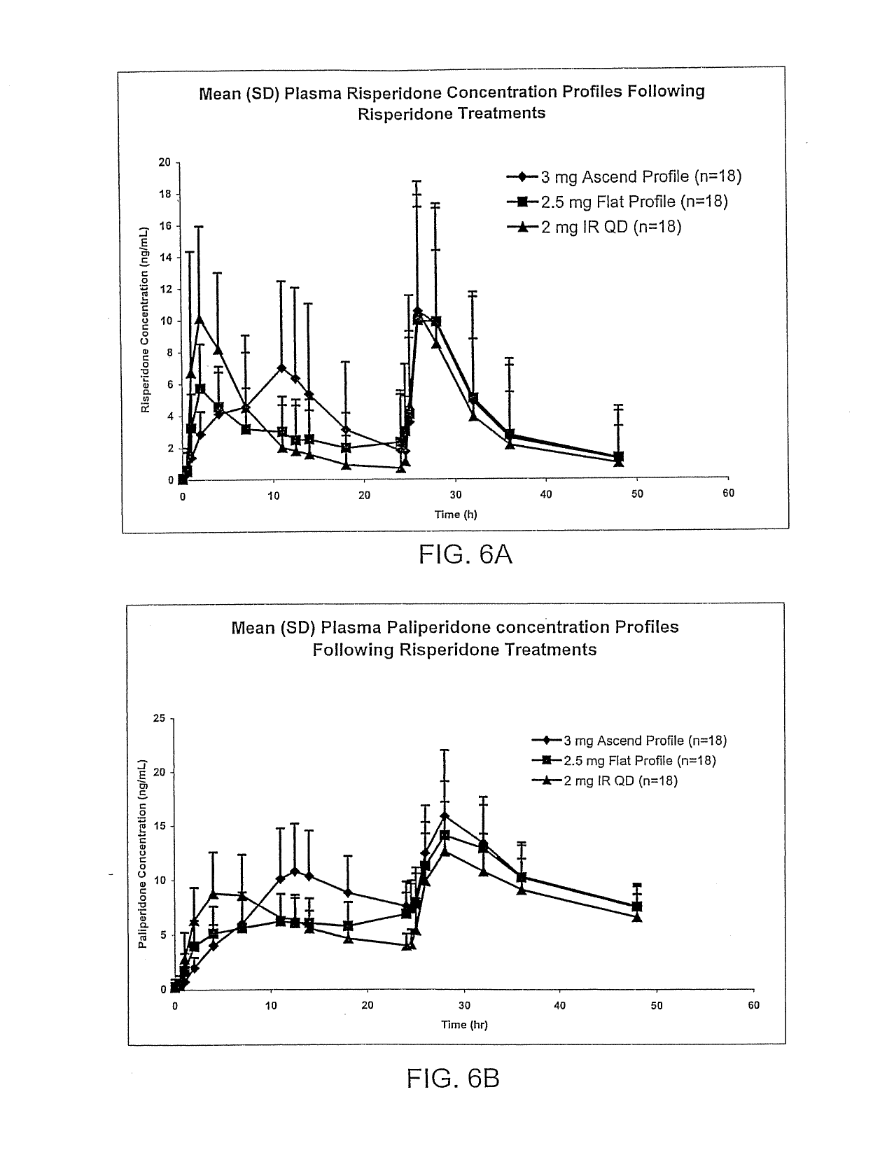

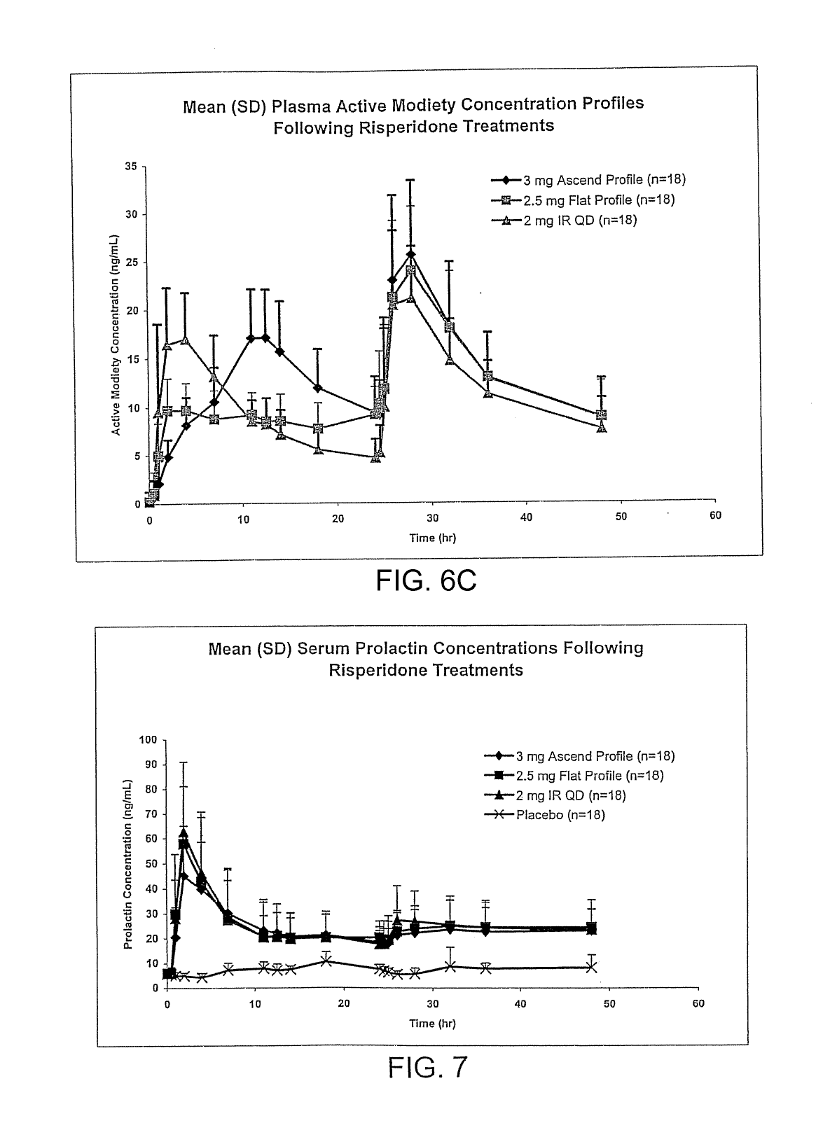

FIGS. 6A, 6B and 6C illustrate the mean risperidone, paliperidone and active moiety plasma concentration profile, respectively, over 48 hours from the study treatments. The mean Cₘₐₓ of risperidone on Day 1 for Ascend and IR were similar. Table 3 provides a tabular summary of the data collected for risperidone, paliperidone and active moiety. Table 4 lists the dose normalized Cmax values from Day 1 when three varying profiles were administered.

TABLE 3
Mean Pharmacokinetic Parameter Values

| | | | |
|---|---|---|---|
| | Ascend 3 mg | Flat 2.5 mg | IR 2 mg |
| PARAMETER | (n = 18) | (n = 18) | (n = 18) |

Risperidone

| | | | |
|---|---|---|---|
| Cₘₐₓ (ng/mL) | 12.1 (8.0) | 11.7 (7.6) | 12.1 (6.4) |
| Regimen^{a} | | | |
| Tₘₐₓ (h) Regimen^{a} | 26.6 (0.92) | 26.7 (1.1) | 10.1 (11.5) |
| Cₘₐₓ (ng/mL) Day 1^{e} | 7.2 (5.6) | 6.1 (2.9) | 11.0 (6.1) |
| Tₘₐₓ (h) Day 1^{e} | 10.6 (2.5) | 3.5 (5.2) | 2.3 (1.0) |
| Cₘₐₓ (ng/mL) Day 2^{f} | 12.1 (8.0) | 11.7 (7.6) | 11.2 (6.6) |
| Tₘₐₓ (h) Day 2^{f} | 2.5 (0.9) | 2.6 (1.1) | 2.4 (1.0) |
| t_{1/2} (h) | 4.4 (3.6) | 5.3 (7.0) | 4.5 (3.6) |
| k (1/h) | 0.23 (0.12) | 0.22 (0.11) | 0.23 (0.12) |
| AUCₜ (ng · h/mL) | 190 (210) | 168 (170) | 158 (159) |
| AUC_{inf} (ng · h/mL) | 212 (264) | 206 (263) | 174 (200) |

Paliperidone

| | | | |
|---|---|---|---|
| Cₘₐₓ (ng/mL) | 16.0 (6.0) | 14.7 (4.6) | 13.0 (4.4) |
| Regimen^{a} | | | |
| Tₘₐₓ (h) Regimen^{a} | 29.2 (5.0) | 30.6 (6.6) | 31.7 (7.6) |
| Cₘₐₓ (ng/mL) Day 1^{e} | 11.3 (4.3) | 7.3 (2.3) | 9.3 (3.8) |
| Tₘₐₓ (h) Day 1^{e} | 14.8 (4.5) | 21.1 (5.3) | 6.2 (5.1) |
| Cₘₐₓ (ng/mL) Day 2^{f} | 16.0 (6.0) | 14.7 (4.6) | 13.0 (4.4) |
| Tₘₐₓ (h) Day 2^{f} | 5.1 (5.0) | 6.5 (6.6) | 7.6 (7.6) |
| t_{1/2} (h)^{b} | 25.2 (8.5) | 28.6 (16.8) | 24.4 (11.1) |
| k (1/h)^{b} | 0.032 (0.015) | 0.030 (0.014) | 0.034 (0.014) |
| AUCₜ (ng · h/mL) | 427 (135) | 376 (115) | 349 (107) |
| AUC_{inf} (ng · h/mL) | 693 (189) | 683 (227) | 570 (144) |

Active Moiety (Risperidone + Paliperidone)

| | | | |
|---|---|---|---|
| Cₘₐₓ (ng/mL) | 27.1 (8.0) | 25.5 (7.6) | 22.8 (6.7) |
| Regimen^{a} | | | |
| Tₘₐₓ (h) Regimen^{a} | 27.1 (1.0) | 27.4 (1.0) | 22.8 (9.3) |
| Cₘₐₓ (ng/mL) Day 1^{e} | 17.8 (5.0) | 11.2 (3.5) | 18.8 (5.9) |
| Tₘₐₓ (h) Day 1^{e} | 12.1 (1.6) | 9.1 (9.2) | 2.9 (1.1) |
| Cₘₐₓ (ng/mL) Day 2^{f} | 27.1 (8.0) | 25.5 (7.6) | 22.8 (6.7) |
| Tₘₐₓ (h) Day 2^{f} | 3.0 (1.0) | 3.3 (1.0) | 2.9 (1.1) |
| AUCₜ (ng · h/mL) | 618 (198) | 545 (161) | 507 (143) |
| AUC_{inf} (ng · h/mL) | 905 (282) | 888 (305) | 744 (199) |

Risperidone/Paliperidone

| | | | |
|---|---|---|---|
| AUC_{inf} Ratio^{c} | 0.38 (0.66) | 0.34 (0.68) | 0.39 (0.75) |
| Excluding 2 Subjects^{d} | 0.19 (0.12) | 0.15 (0.06) | 0.19 (0.08) |

^{a}Cmax and Tmax estimated over the entire regimen (Day 1 and Day 2; dosing profile described in Table 2)
^{b}n = 14, 15, and 15 respectively for Ascend, Flat, and IR Treatment
^{c}All available subjects: n = 19, 21, and 21 respectively for Ascend, Flat, and IR Treatment
^{d}Subjects 114 and 122, who appear to be poor metabolizers, are excluded
^{e}Cmax and Tmax estimated over the Day 1 concentration curve obtained with the dosing profile described in Table 2
^{f}Cmax and Tmax estimated over the Day 2 concentration curve obtained with the dosing profile described in Table 2
Note:
Cmax and Tmax values estimated from concentration profile obtained by the sum of risperidone and paliperidone.
AUC values estimated by sum of AUC values of risperidone and paliperidone.

TABLE 4
Dose Normalized Day 1Pharmacokinetic Parameter Values

| | | | |
|---|---|---|---|
| | Ascend 3 mg | Flat 2.5 mg | IR 2 mg |
| PARAMETER | (n = 18) | (n = 18) | (n = 18) |

Risperidone

| | | | |
|---|---|---|---|
| Cₘₐₓ (ng/mL) Day 1 | 7.2 (5.6) | 6.1 (2.9) | 11.0 (6.1) |
| Dose normalized Cmax | 2.4 (1.9) | 2.4 (1.2) | 5.5 (3.1) |

Paliperidone

| | | | |
|---|---|---|---|
| Cₘₐₓ (ng/mL) Day 1 | 11.3 (4.3) | 7.3 (2.3) | 9.3 (3.8) |
| Dose normalized Cmax | 3.8 (1.4) | 2.9 (0.9) | 4.7 (1.9) |

Active Moiety (Risperidone + Paliperidone)

| | | | |
|---|---|---|---|
| Cₘₐₓ (ng/mL) Day 1 | 17.8 (5.0) | 11.2 (3.5) | 18.8 (5.9) |
| Dose normalized Cmax | 5.9 (1.7) | 4.5 (1.4) | 9.4 (3.0) |

FIG. 7 illustrates the mean prolactin profile associated with the three treatments. Table 5 is a summary of the prolactin data collected. The Ascend treatment had the lowest elevation (as seen by the Cₘₐₓ value) on Day 1 followed by the Flat and IR treatments despite the higher dose on Day 1.

TABLE 5
Mean (SD) Prolactin Parameters (n = 18)

| | Ascend | Flat | IR | |
|---|---|---|---|---|
| Parameter | 3 mg | 2.5 mg | 2 mg | Placebo |
| Cₘₐₓ (ng/mL) | 51.6 (29.4) | 58.9 (27.9) | 63.3 (27.7) | 12.0 (3.1) |
| Day 1 | | | | |
| Tₘₐₓ (h) Day 1 | 2.3 (1.0) | 1.8 (0.4) | 1.8 (0.4) | 17.3 (4.4) |
| Cₘₐₓ (ng/mL) | 25.0 (11.2) | 26.4 (11.3) | 28.3 (13.2) | 12.3 (7.5) |
| Day 2 | | | | |
| Tₘₐₓ (h) Day 2 | 14.1 (8.3) | 12.2 (8.0) | 8.4 (9.1) | 10.8 (7.7) |
| AUC₀₋₄₈ | 1141 (487) | 1204 (520) | 1220 (501) | 353 (75) |
| (ng · h/mL) | | | | |

A drop in systolic blood pressure greater than 20 mm Hg or dizziness/faintness was seen in 9.5%, 17.4%, 17.4%, and 0.0% of the subjects for the Ascend, Flat, IR and placebo treatments, respectively. Thus, on day 1 despite the higher dose in the Ascend treatment, the incidence of these effects was lower than with the other two active treatments. On day 2, no subject in the Ascend treatment had a drop in systolic blood pressure greater than 20 mm Hg or experienced dizziness/faintness compared with 8.7% and 13.0% for the IR and Flat treatments, respectively. This suggests that tolerance to orthostatic hypotension could develop within a day, when the plasma concentrations rise slowly.

Accordingly, the Ascend treatment with the slowly ascending plasma profile is unexpectedly better tolerated with respect to orthostatic hypotension and to result in less prolactin elevation than either the Flat or IR plasma profiles.

### Example 3

### A Pharmacokinetic-Pharmacodynamic Study to Evaluate Various Dosing Regimens of Paliperidone

This study was conducted to evaluate pharmacodynamic effects (postural changes in blood pressure and measurements of prolactin serum concentration) following three different dosing profiles of paliperidone. Twenty-five of twenty-seven volunteers completed the study. Only 24 subjects had paliperidone and prolactin concentration measured in all three treatments.

This study utilized three oral dosing schedules of paliperidone and a placebo.
1. Ascend Profile (total dose of 5.5 mg paliperidone)—total of 3.5 mg in divided doses administered as a solution over 20.25 hours on day 1 followed by a single 2 mg solution on day 2.
2. Flat profile (total dose of 4.5 mg paliperidone)—total of 2.5 mg in divided doses administered as a solution over 20.25 hours on day 1, followed by a single 2 mg solution on day 2.
3. IR profile (total dose of 4 mg paliperidone)—2 mg in solution on 2 consecutive days.
4. Placebo solution administered at each dosing time

Table 6, below provides the dosing schedule for each system. All subjects received all four treatments randomly. There was a 6-to-10-day washout period between treatments. The washout period began after the last dose in each treatment period.

TABLE 6
Study Medication Schedule (mg)

Treatment

| Dose | Dose | A | B | C | D |
|---|---|---|---|---|---|
| Number | Hour | ASCEND | FLAT | IR | Placebo |
| 1 | 0 | 0.25 | 1.0 | 2.0 | 0.0 |
| 2 | 2 | 0.25 | 0.0 | 0.0 | 0.0 |
| 3 | 2.75 | 0.0 | 0.25 | 0.0 | 0.0 |
| 4 | 3.75 | 0.25 | 0.0 | 0.0 | 0.0 |
| 5 | 5.25 | 0.25 | 0.25 | 0.0 | 0.0 |
| 6 | 6.5 | 0.25 | 0.0 | 0.0 | 0.0 |
| 7 | 8.25 | 0.25 | 0.25 | 0.0 | 0.0 |
| 8 | 10.00 | 0.25 | 0.0 | 0.0 | 0.0 |
| 9 | 11.25 | 0.25 | 0.25 | 0.0 | 0.0 |
| 10 | 12.75 | 0.25 | 0.0 | 0.0 | 0.0 |
| 11 | 14.5 | 0.25 | 0.0 | 0.0 | 0.0 |
| 12 | 15.75 | 0.25 | 0.25 | 0.0 | 0.0 |
| 13 | 17.25 | 0.25 | 0.0 | 0.0 | 0.0 |
| 14 | 18.75 | 0.25 | 0.0 | 0.0 | 0.0 |
| 15 | 20.25 | 0.25 | 0.25 | 0.0 | 0.0 |
| 16 | 24 | 12.0 | 2.0 | 2.0 | 0.0 |

| | | | | |
|---|---|---|---|---|
| Total Dose (mg) | 5.5 | 4.5 | 4.0 | 0.0 |

Blood samples were collected from each subject during each treatment period for measurement of paliperidone concentrations at the following times: 0 (pre-dose), 0.5, 1, 2, 4, 7, 11, 14, 18, 21, 24, 24.5, 25, 26, 28, 32, 36 and 48 h after treatment initiation.

Blood samples for prolactin concentration were collected as described above for the pharmacokinetic blood samples. Postural changes in blood pressure and heart rate were assessed during each treatment period at the following times: 0 (pre-dose), 20 minutes (min), 50 min, at 50 minutes after Hours 1, 3, 4, 6, 7, 8, 9, 10, 12, 13, 23, 24, 25, 27, 31, 35, and 47, as well as 17 h 20 min, 21 h, and 24 h 20 min after treatment initiation. An automated blood pressure monitor was used to collect blood pressure while supine and 2 and 3 minutes after standing up.

Dizziness and fainting symptoms after standing were assessed as described in Example 2.

Pharmacokinetic parameters AUCₜ, AUC_{inf}, Cₘₐₓ, Tₘₐₓ, and t_{1/2} and prolactin parameters, AUC₄₈, Cₘₐₓ, and Tₘₐₓ, were calculated for paliperidone for each treatment and subject.

AUCt, Cmax, and Tmax were calculated for prolactin for each treatment and subject for each day.

The percentage of subjects with >20 mm Hg drop in systolic blood pressure (SBP) at 3 minutes of standing or with symptoms of orthostatic hypotension (dizziness or faintness) was summarized for Days 1 and 2

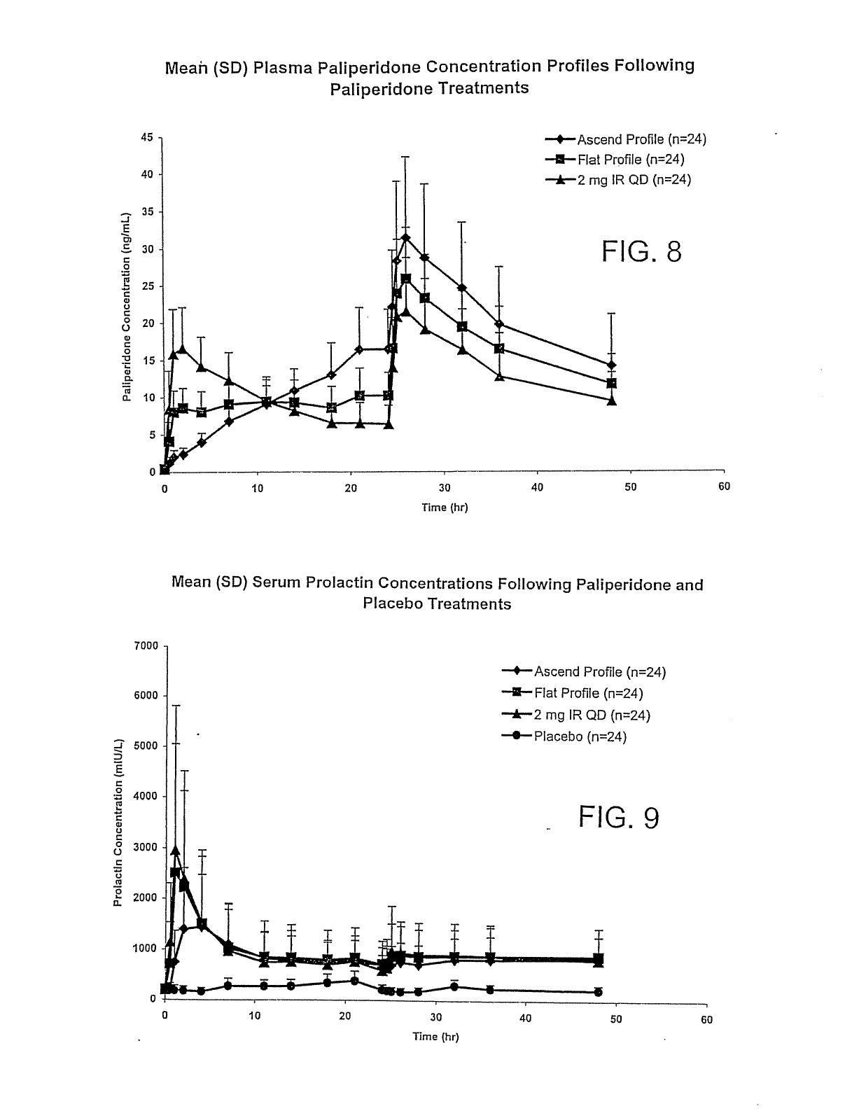

FIG. 8 illustrates the mean paliperidone blood plasma concentration profile over 48 hours. The mean Cₘₐₓ of paliperidone on Day 1 was significantly greater for Ascend and IR and the Flat Cₘₐₓ was approximately 60% of the IR Cₘₐₓ. Table 7 provides a tabular summary of the data collected.

TABLE 7
Pharmacokinetic Values Following Three Active Treatments

| N = 24 | Paliperidone Treatment Mean (SD) | | |
|---|---|---|---|
| Paliperidone | Ascend | Flat | IR |
| Concentration | 3.5 mg | 2.5 mg | 2 mg |
| Cₘₐₓ (ng/mL) | 32.0 (10.5) | 26.5 (7.3) | 22.6 (7.0) |
| Regimen^{a} | | | |
| Tₘₐₓ (h) Regimen^{a} | 26.0 (0.75) | 26.0 (0.7) | 25.0 (5.3) |
| Cₘₐₓ Day 1 (ng/mL)^{b} | 17.0 (5.5) | 10.6 (3.7) | 17.6 (5.4) |
| Dose normalized | 4.9 (1.6) | 4.2 (1.5) | 8.8 (2.7) |
| Cmax Day 1^{b} | | | |
| Cₘₐₓ Day 2 (ng/mL)^{c} | 32.0 (10.5) | 26.5 (7.3) | 22.3 (7.5) |
| AUC_{inf} (ng.h/mL) | 1194 (648) | 969 (317) | 860 (350) |
| Tₘₐₓ Day 1 (h)^{b} | 22.7 (1.5) | 19.4 (6.6) | 1.7 (1.0) |
| Tₘₐₓ Day 2 (h)^{c} | 2.1 (0.8) | 2.0 (0.7) | 2.1 (1.5) |
| t_{1/2} (h)^{d} | 20.4 (6.2) | 20.5 (4.0) | 21.3 (5.6) |

^{a}Cmax and Tmax estimated over the entire regimen (Day 1 and Day 2; dosing profile described in Table 6)
^{b}Cmax and Tmax estimated over the Day 1 concentration curve obtained with the dosing profile described in Table 6
^{c}Cmax and Tmax estimated over the Day 2 concentration curve obtained with the dosing profile described in Table 6
^{d}n = 20 for t_{1/2} for Flat and IR treatments.

Table 8 provides a summary of orthostatic hypotension and symptoms of dizziness or faintness associated with the four treatments.

TABLE 8
Drop of >20 mmHg SBP or Symptoms of Dizziness or Faintness

| | Ascend | Flat | IR | Placebo |
|---|---|---|---|---|
| Day 1 | 54% | 42% | 59% | 23% |
| Day 2 | 50% | 35% | 41% | 15% |

FIG. 9 illustrates the mean prolactin profile associated with the three treatments. Table 9 is a tabular summary of the prolactin data collected.

TABLE 9
Serum Prolactin Values for the Three Active Treatments and Placebo

| Prolactin | Mean (SD), n = 24 | | | |
|---|---|---|---|---|
| Parameters | Ascend | Flat | IR | Placebo |
| Cₘₐₓ Day 1 (mIU/L) | 1594 (1194) | 2614 (2525) | 2956 (2857) | 449 (195) |
| Cₘₐₓ Day 2 (mIU/L) | 877 (443) | 982 (632) | 1059 (867) | 332 (117) |
| Tₘₐₓ Day 1 (h) | 3.6 (1.7) | 1.2 (0.4) | 1.0 (0.2) | 16.7 (5.2) |
| Tₘₐₓ Day 2 (h) | 15.6 (8.4) | 9.7 (9.0) | 8.3 (9.5) | 10.9 (5.5) |
| AUC₀₋₄₈ (mIU · h/L) | 41,012 (22,998) | 46,077 (33,105) | 45,370 (33,083) | 12,258 (4,379) |

The study concluded that the Flat treatment provided the lowest incidence of orthostatic hypotension. Ascend treatment resulted in an incidence of orthostatic hypotension slightly lower than the IR treatment on Day 1, despite a 75% higher Ascend dose. Ascend also resulted in less prolactin elevation as compared to the other treatments.

### Example 4

### Comparison of Pharmacodynamic Effects of Risperidone and Paliperidone

This study was conducted to evaluate pharmacodynamic effects (postural changes in blood pressure and heart rate and measurements of prolactin serum concentration) following different dosing profiles of paliperidone and risperidone and compared to IR. Twenty-five volunteers completed the study.

This study utilized four oral dosing schedules of paliperidone and risperidone and a placebo.
1. Risperidone Ascend—4 Profile (total dose of 6.0 mg risperidone)—4.0 mg risperidone in divided doses administered as a solution over 21 hours on day 1 followed by a single 2 mg solution on day 2.
2. Paliperidone Ascend—4 Profile (total dose of 6.0 mg paliperidone)—4.0 mg paliperidone in divided doses administered as a solution over 21 hours on day 1, followed by a single 2 mg solution on day 2.
3. Paliperidone Ascend—2 Profile (total dose of 4.0 mg paliperidone)—2.0 mg paliperidone in divided doses administered as a solution over 21 hours on day 1, followed by a single 2 mg solution on day 2.
4. Risperidone IR profile was a total dose of 4 mg risperidone—2 mg in solution on 2 consecutive days.
5. Placebo solution administered at each dosing time

Table 10, below provides the dosing schedule for each system. All subjects received all five treatments randomly. There was a 6-to-10-day washout period between treatments. The washout period began after the last dose in each treatment period.

TABLE 10
Study Medication Schedule (mg)

| | | Treatment | | | | |
|---|---|---|---|---|---|---|
| | | A | B | C | D | |
| Dose | Dose | Risp | Pali | Pali | Risp | E |
| Number | Hour | ASCEND-4 | ASCEND-4 | ASCEND-2 | IR-2 | PLACEBO |
| 1 | 0 | 0.5 | 0.5 | 0.25 | 2.0 | 0.0 |
| 2 | 2 | 0.25 | 0.25 | 0.125 | 0.0 | 0.0 |
| 3 | 4 | 0.25 | 0.25 | 0.125 | 0.0 | 0.0 |
| 4 | 5.75 | 0.25 | 0.25 | 0.125 | 0.0 | 0.0 |
| 5 | 7.25 | 0.25 | 0.25 | 0.125 | 0.0 | 0.0 |
| 6 | 8.75 | 0.25 | 0.25 | 0.125 | 0.0 | 0.0 |
| 7 | 10.25 | 0.25 | 0.25 | 0.125 | 0.0 | 0.0 |
| 8 | 11.75 | 0.25 | 0.25 | 0.125 | 0.0 | 0.0 |
| 9 | 13.25 | 0.25 | 0.25 | 0.125 | 0.0 | 0.0 |
| 10 | 14.75 | 0.25 | 0.25 | 0.125 | 0.0 | 0.0 |
| 11 | 16 | 0.25 | 0.25 | 0.125 | 0.0 | 0.0 |
| 12 | 17.25 | 0.25 | 0.25 | 0.125 | 0.0 | 0.0 |
| 13 | 18.5 | 0.25 | 0.25 | 0.125 | 0.0 | 0.0 |
| 14 | 19.75 | 0.25 | 0.25 | 0.125 | 0.0 | 0.0 |
| 15 | 21 | 0.25 | 0.25 | 0.125 | 0.0 | 0.0 |
| 16 | 24 | 2.0 | 2.0 | 2.0 | 2.0 | 0.0 |

| | | | | | |
|---|---|---|---|---|---|
| Total Dose | 6.0 | 6.0 | 4.0 | 4.0 | 0.0 |
| (mg) | | | | | |

Blood samples were collected from each subject during each treatment period for measurement of concentrations of paliperidone or risperidone at the following times: 0 (pre-dose), 0.5, 1, 2, 4, 8, 12, 17, 22, 24, 24.5, 25, 26, 30, 36, and 48 h post treatment initiation.

Blood samples for prolactin concentration were collected as described above for the pharmacokinetic blood samples. Postural changes in blood pressure and heart rate were assessed during each treatment period at the following times: 0 (pre-dose), 20 minutes (min), 50 min, at 50 min after Hours 1, 3, 4, 6, 7, 8, 9, 10, 12, 13, 16, 21, 23, 24, 25, 27, 31, 35, and 47 plus at 24 h 20 min posttreatment initiation. An automated blood pressure monitor was used to collect blood pressure while supine and 2 and 3 minutes after standing up.

Dizziness and fainting symptoms after standing were assessed as described in Example 2.

PK parameters AUCₜ, AUC_{inf}, Cₘₐₓ, Tₘₐₓ, and t_{1/2} were calculated for paliperidone for each treatment and subject. Risperidone and active moiety (risperidone+paliperidone) parameters were also estimated for the two risperidone treatments. Paliperidone is the active moiety following paliperidone treatments. AUCt, Cmax, and Tmax were calculated for prolactin for each treatment and subject for each day.

The percentage of subjects with >20 mm Hg drop in systolic blood pressure (SBP) at 3 minutes of standing or with symptoms of orthostatic hypotension (dizziness or faintness) was summarized for Days 1 and 2

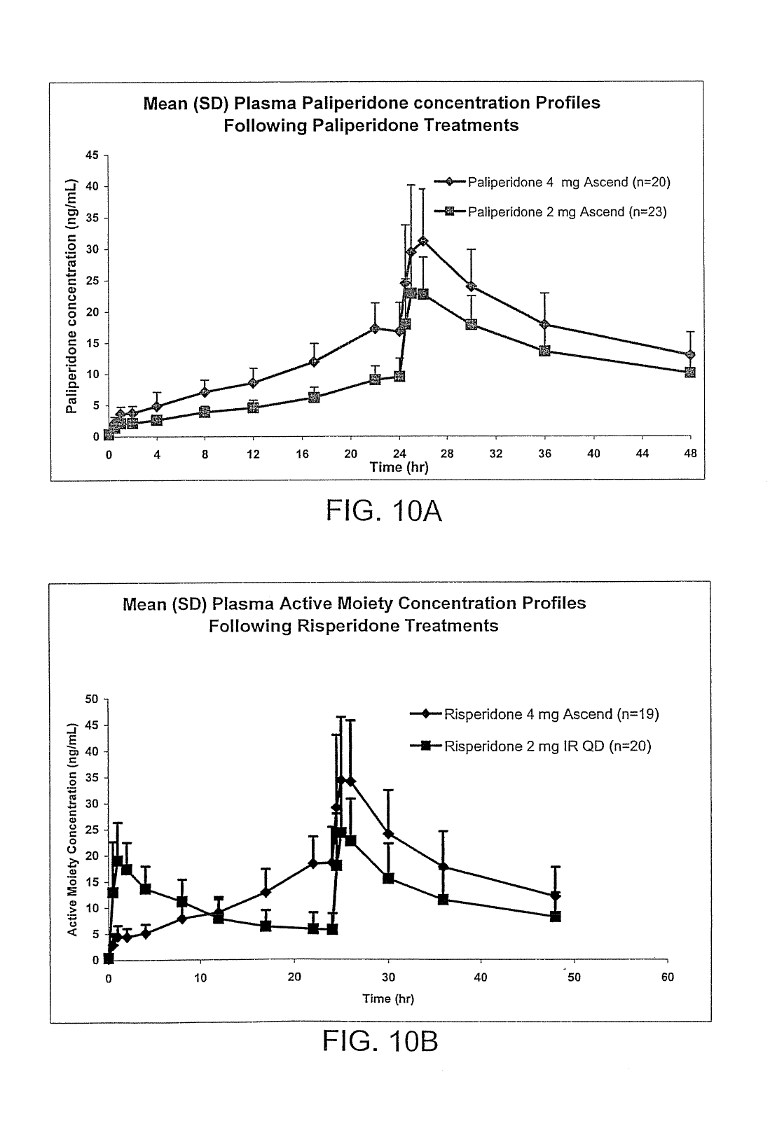

FIGS. 10 A and 10B illustrate the mean active moiety plasma concentration profile over 48 hours for the paliperidone and risperidone treatments, respectively, (paliperidone is the active moiety for paliperidone treatments and sum of risperidone and paliperidone is the active moiety for risperidone treatments). Table 11 is a tabular summary of the blood plasma concentration pharmacokinetic data for the paliperidone treatments. Table 12 is a tabular summary of the blood plasma concentration pharmacokinetic data for the risperidone treatments. Mean Cₘₐₓ values were similar for the active moiety (risperidone+paliperidone) following Risperidone Ascend-4 and Risperidone IR-2, and for the active moiety (paliperidone) following Paliperidone Ascend-4 on Day 1 (19.5, 21.3, 17.6 ng/mL, respectively). Cₘₐₓ for the active moiety (paliperidone) following Paliperidone Ascend-2 on Day 1 was 9.3 ng/mL, approximately half of Paliperidone Ascend-4 Cₘₐₓ.

TABLE 11
Pharmacokinetic Values Following Four Active Treatments

| | | |
|---|---|---|
| PARAMETER | Paliperidone | Paliperidone |
| N = 22 | Ascend 4 mg | Ascend 2 mg |

| | |
|---|---|
| Paliperidone (Active Moiety) | Mean (SD) |

| | | |
|---|---|---|
| Cₘₐₓ (ng/mL) Regimen^{a} | 31.7 (9.2) | 23.3 (6.9) |
| Tₘₐₓ (h) Regimen^{a} | 25.6 (0.5) | 25.5 (0.6) |
| Cₘₐₓ (ng/mL) Day 1^{b} | 17.6 (4.4) | 9.3 (2.7) |
| Tₘₐₓ (h) Day 1^{b} | 23.1 (1.0) | 22.3 (3.3) |
| Cₘₐₓ (ng/mL) Day 2^{c} | 31.7 (9.2) | 23.3 (6.9) |
| Tₘₐₓ (h) Day 2^{c} | 1.6 (0.5) | 1.5 (0.6) |
| T_{1/2} (h) | 20.2 (5.2) | 20.1 (5.0) |
| AUCₜ (ng · h/mL) | 686.2 (174.7) | 449.0 (122.2) |
| AUC_{inf} (ng · h/mL) | 1063.2 (315.1) | 727.7 (239.7) |

^{a}Cmax and Tmax estimated over the entire regimen (Day 1 and Day 2; dosing profile described in Table 10)
^{b}Cmax and Tmax estimated over the Day 1 concentration curve obtained with the dosing profile described in Table 10
^{c}Cmax and Tmax estimated over the Day 2 concentration curve obtained with the dosing profile described in Table 10
Note:
Subjects received 2 mg IR paliperidone on Day 2.

TABLE 12
Pharmacokinetic Values Following Four Active Treatments

| | Risperidone | Risperidone |
|---|---|---|
| Parameter | Ascend 4 mg | IR 2 mg |
| (N = 22) | Mean (SD) | |
| Risperidone Concentration | | |
| Cₘₐₓ (ng/mL) Regimen^{a} | 18.8 (14.6) | 16.6 (11.0) |
| Tₘₐₓ (h) Regimen^{a} | 25.3 (0.6) | 10.8 (12.3) |
| Cₘₐₓ (ng/mL) Day 1^{g} | 8.6 (7.8) | 14.9 (8.2) |
| Tₘₐₓ (h) Day 1^{g} | 21.2 (4.7) | 1.0 (0.4) |
| Cₘₐₓ (ng/mL) Day 2^{h} | 18.8 (14.6) | 15.0 (11.2) |
| Tₘₐₓ (h) Day 2^{h} | 1.3 (0.6) | 1.1 (0.4) |
| T_{1/2} (h) | 6.4 (6.2)^{b} | 5.8 (5.5)^{c} |
| AUCₜ (ng · h/mL) | 289.1 (316.5) | 237.5 (271.5) |
| AUC_{inf} (ng · h/mL) | 367.4 (471.5) | 286.5 (386.9) |
| Paliperidone Concentration | | |
| Cₘₐₓ (ng/mL) Regimen^{a} | 17.1 (8.6) | 11.0 (5.2) |
| Tₘₐₓ (h) Regimen^{a} | 29.9 (7.8) | 29.7 (7.2) |
| Cₘₐₓ (ng/mL) Day 1^{g} | 11.1 (5.9) | 7.6 (3.8) |
| Tₘₐₓ (h) Day 1^{g} | 23.1 (1.0) | 7.7 (8.5) |
| Cₘₐₓ (ng/mL) Day 2^{h} | 17.1 (8.6) | 11.0 (5.2) |
| Tₘₐₓ (h) Day 2^{h} | 5.9 (7.8) | 5.7 (7.2) |
| T_{1/2} (h) | 23.5 (9.7)^{d} | 22.1 (6.4)^{e} |
| AUCₜ (ng · h/mL) | 418.6 (201.1) | 291.9 (129.6) |
| AUC_{inf} (ng · h/mL) | 727.9 (388.6) | 473.9 (216.0) |
| Active Moiety (Risperidone + | | |
| Paliperidone)ⁱ | | |
| Cₘₐₓ (ng/mL) Regimen^{a} | 35.3 (12.1) | 25.3 (9.6) |
| Tₘₐₓ (h) Regimen^{a} | 25.7 (1.1) | 20.9 (9.7) |
| Cₘₐₓ (ng/mL) Day 1^{g} | 19.5 (6.3) | 21.3 (7.6) |
| Tₘₐₓ (h) Day 1^{g} | 22.5 (0.9) | 1.1 (0.4) |
| Cₘₐₓ (ng/mL) Day 2^{h} | 35.3 (12.1) | 25.1 (9.5) |
| Tₘₐₓ (h) Day 2^{h} | 1.7 (1.1) | 1.3 (0.5) |
| AUCₜ (ng · h/mL) | 707.7 (232.5) | 529.4 (219.8) |
| AUC_{inf} (ng · h/mL) | 1095.2 (430.2) | 760.4 (349.0) |
| Ratio of Risperidone/Paliperidone | | |
| AUC_{inf} Ratio | 1.22 (2.13) | 1.13 (1.82) |
| AUC_{inf} Ratio^{f} | 0.14 (0.06)^{f} | 0.19 (0.10)^{f} |

^{a}Cmax and Tmax estimated over the entire regimen (Day 1 and Day 2; dosing profile described in Table 10)
^{b}n = 21,
^{c}n = 20,
^{d}n = 17,
^{e}n = 15
^{f}AUC_{inf} Ratio calculated with n = 17 excluding the 5 subjects who possibly were poor metabolizers.
^{g}Cmax and Tmax estimated over the Day 1 concentration curve obtained with the dosing profile described in Table 10
^{h}Cmax and Tmax estimated over the Day 2 concentration curve obtained with the dosing profile described in Table 10
ⁱCmax and Tmax values estimated from the concentration profile of the sum of risperidone and paliperidone.
AUC values estimated by sum of AUC values of risperidone and paliperidone.
Note:
Subjects in each active treatment received 2 mg IR risperidone on Day 2.

TABLE 13
Dose Normalized Day 1 Pharmacokinetic Parameter Values

| | | | | |
|---|---|---|---|---|
| | Paliperidone | Paliperidone | | |
| | Ascend | Ascend | Risperidone | Risperidone |
| | 4 mg* | 2 mg* | Ascend 4 mg | IR 2 mg |
| PARAMETER | (n = 22) | (n = 22) | (n = 22) | (n = 22) |

Risperidone

| | | | | |
|---|---|---|---|---|
| Cₘₐₓ (ng/mL) | NA | NA | 8.6 (7.8) | 14.9 (8.2) |
| Day 1 | | | | |
| Dose | NA | NA | 2.2 (2.0) | 7.5 (4.1) |
| normalized | | | | |
| Cmax | | | | |

Paliperidone

| | | | | |
|---|---|---|---|---|
| Cₘₐₓ (ng/mL) | 17.6 (4.4) | 9.3 (2.7) | 11.1 (5.9) | 7.6 (3.8) |
| Day 1 | | | | |
| Dose | 4.4 (1.1) | 4.7 (1.4) | 2.8 (1.5) | 3.8 (1.9) |
| normalized | | | | |
| Cmax | | | | |

Active Moiety (Risperidone + Paliperidone)

| | | | | |
|---|---|---|---|---|
| Cₘₐₓ (ng/mL) | NA | NA | 19.5 (6.3) | 21.3 (7.6) |
| Day 1 | | | | |
| Dose | NA | NA | 4.9 (1.6) | 10.7 (3.8) |
| normalized | | | | |
| Cmax | | | | |

For Paliperidone treatments Active moiety is paliperidone

Table 14 provides a summary of orthostatic hypotension and symptoms of dizziness or faintness associated with the five treatments.

TABLE 14
>20 mmHg Drop in SBP or Symptoms of Dizziness or Faintness

| | Risp. | Risp. | Pali. | Pali. | |
|---|---|---|---|---|---|
| | Ascend-4 | IR-2 | Ascend-4 | Ascend-2 | Placebo |
| | N = 22 | N = 22 | N = 21 | N = 25 | N = 27 |
| Day 1 | 32% | 46% | 29% | 32% | 7% |
| Day 2 | 19% | 38% | 24% | 36% | 15% |

The percentage of subjects with orthostatic effects following Risperidone and Paliperidone Ascend-4 treatments were comparable on Day 1 and, despite a dose two times greater, were less than the percentage following Risperidone IR-2. The percentage of subjects with orthostatic effects on Day 1 following Paliperidone Ascend-2 was similar to that of Paliperidone Ascend-4 even though the Ascend-2 treatment Day 1 dose was half that of Ascend-4. However, a majority of the subjects had milder responses following Paliperidone Ascend-2 treatment. On Day 2, even though the active moiety concentration was higher, Risperidone Ascend-4 and Paliperidone Ascend-4 had a lower or similar Day 2 incidence than Risperidone IR-2. These results may suggest the development of tolerance to orthostatic effects with the Ascend-4 treatments.

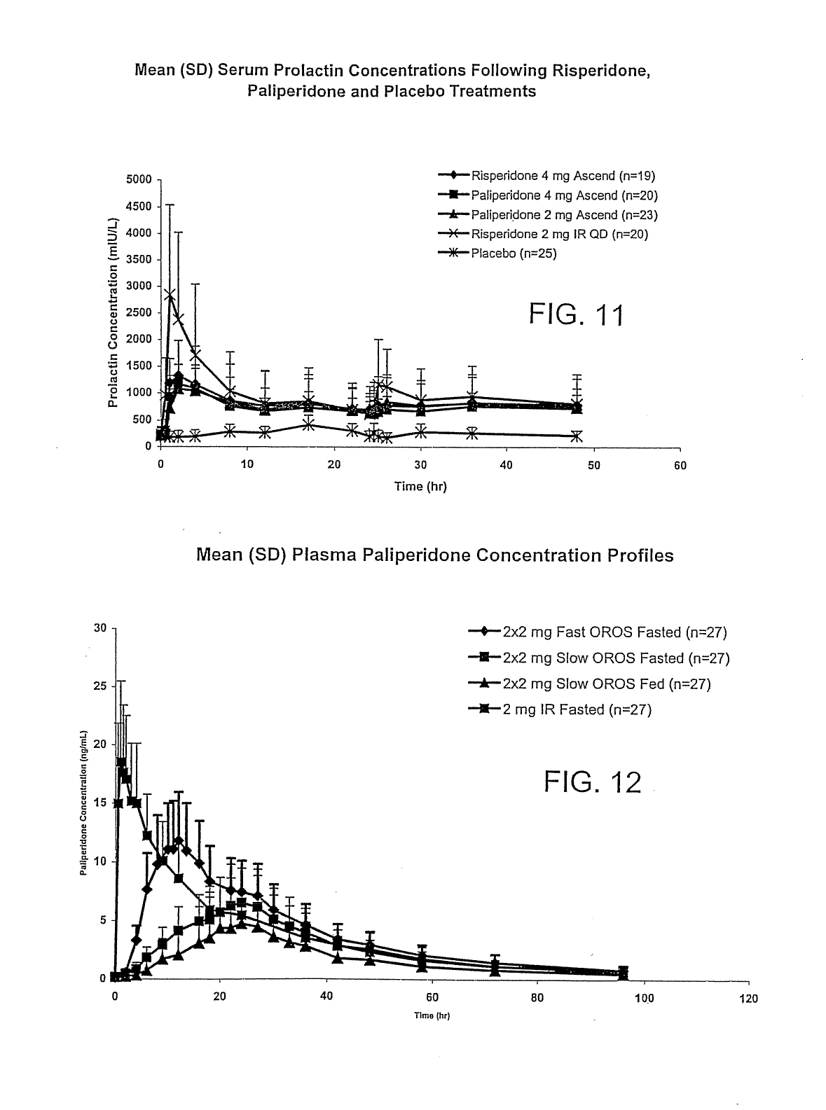

FIG. 11 illustrates the blood plasma prolactin concentration over 48 hours associated with the treatments. Table 12 is a tabular summary of serum prolactin values for the four active treatments and placebo. Prolactin day 1 Cₘₐₓ and prolactin AUC₀₋₄₈ were lower for all three Ascend treatments than for risperidone IR—2 mg.

TABLE 15
Serum Prolactin Values for the Four Active Treatments and Placebo

| | Risp. | Risp. | Pali. | Pali. | |
|---|---|---|---|---|---|
| Prolactin | Ascend-4 | IR-2 | Ascend-4 | Ascend-2 | Placebo |
| Parameter | N = 19 | N = 20 | N = 20 | N = 23 | N = 25 |
| Cₘₐₓ Day 1 | 1543 (765) | 2848 (1710) | 1271 (422) | 1203 (513) | 455 (167) |
| (mIU/L) | | | | | |
| Cₘₐₓ Day 2 | 850 (538) | 1208 (864) | 780 (250) | 873 (506) | 348 (185) |
| (mIU/L) | | | | | |
| Tₘₐₓ Day 1 | 2.5 (3.6) | 1.0 (0.2) | 2.5 (1.7) | 5.5 (6.8) | 16.3 (3.5) |
| (h) | | | | | |
| Tₘₐₓ Day 2 | 10.6 (6.3) | 2.5 (3.3) | 12.3 (6.2) | 6.9 (8.0) | 7.4 (5.4) |
| (h) | | | | | |
| AUC₀₋₄₈ | 39,520 (25,778) | 47,965 (30,007) | 35,637 (11,538) | 37,685 (19,448) | 12,488 (5,190) |
| (mIU · h/L) | | | | | |

Risp = Risperidone;
Pali = Paliperidone

The study concluded that Ascend treatment results in development of tolerance to orthostatic effects within the first day of dosing. The conclusion was based on the observation of a lower percentage of subjects with orthostatic effects on Day 1 with Ascend-4 risperidone and paliperidone treatments than Risperidone IR-2. This occurred even though the Ascend doses were twice that of IR-2. On Day 2, although active moiety plasma concentrations were higher following the Risperidone Ascend-4 and Paliperidone Ascend-4 treatments, the percentage of subjects experiencing orthostatic effects was lower or similar to Risperidone IR-2.

In addition the Ascend treatments were associated with lower prolactin elevation than risperidone IR-2.

### Example 5

### 2 mg Risperidone Osmotic Module Formulation

First, a push composition was prepared as follows: first, a binder solution was prepared. 4.3 kg of hydroxypropyl methylcellulose identified as 2910 was dissolved in 38.7 kg of water. Then, 36 kg of sodium chloride and 0.36 kg of ferric oxide were sized using a Quadro Comil with a 21-mesh screen. Then, the screened materials, 2.4 kg of hydroxypropyl methylcellulose identified as 2910 and 76.44 kg of polyethylene oxide (approximately 7,000,000 molecular weight) were added to a fluid bed granulator bowl. The dry materials were fluidized and mixed while 36 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 60 g of butylated hydroxytoluene and lubricated with 1.14 kg of stearic acid.

Next, the barrier layer was prepared as follows: 3 kg of polyvinyl acetate/povidone and 3 kg of microfine wax, grade MF-2JH were charged to the bowl of the Hobart mixer. The dry components were mixed for 5 minutes. Then, water was added to the mixing bowl at a constant rate to reach acceptable granulation results. The resulting wet granulation was manually pressed through a 16-mesh screen and dried at 50 Deg C. to an acceptable moisture level. Finally, the dry granulation was manually sized using a 16-mesh screen

Next, the push and the barrier layer granulations were compressed into bilayer arrangements. 85 mg of barrier layer granulation was compressed with 270 mg of push layer granulation using the rotary tablet press with 0.278″(7 mm) tooling.

Next, the osmotic module was assembled as follows: bilayer arrangements of push and barrier layers were inserted to a depth of 0.525 inches into the size 0, transparent HPMC capsule body.

Next, the assembled osmotic modules were coated with a semi-permeable wall. The wall forming composition comprised 90% cellulose acetate having a 39.8% acetyl content and 10% poloxamer 188. The wall-forming composition was dissolved in acetone. The wall-forming composition was sprayed onto and around the bilayered arrangements in a pan coater until approximately 60 mg of membrane was applied to each tablet.

Next, a 20 mil (0.51 mm)) exit passageway was drilled through the semi-permeable wall to connect the drug layer with the exterior of the dosage system. The residual solvent was removed by drying at 45° C. and 45% RH for 24 hours followed by drying at 45° C. and ambient humidity for additional 24 hours.

Next, a liquid drug layer composition was prepared as follows: 29.862 g of polysorbate 80 was weighed into the glass jar. Then, 15 mg of butylated hydroxytoluene was mixed with polysorbate 80 for 30 seconds. Finally, 0.123 g of risperidone was added into solution, pre-mixed with a spatula for 30 seconds and then mixed on a stirring plate for 20 hours.

Next, the empty compartment of the osmotic module was filled with a liquid drug layer using syringe. Approximately 500 mg of the liquid drug layer was dispensed into each osmotic module.

### Example 6

### 2 mg Paliperidone Osmotic Module Formulation

First, a push composition was prepared as follows: first, a binder solution was prepared. 4.3 kg of hydroxypropyl methylcellulose identified as 2910 was dissolved in 38.7 kg of water. Then, 36 kg of sodium chloride and 0.36 kg of ferric oxide were sized using a Quadro Comil with a 21-mesh screen. Then, the screened materials, 2.4 kg of hydroxypropyl methylcellulose identified as 2910 and 76.44 kg of polyethylene oxide (approximately 7,000,000 molecular weight) were added to a fluid bed granulator bowl. The dry materials were fluidized and mixed while 36 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 60 g of butylated hydroxytoluene and lubricated with 1.14 kg of stearic acid.

Next, the barrier layer was prepared as follows: 3 kg of polyvinyl acetate/povidone and 3 kg of microfine wax, grade MF-2JH were charged to the bowl of the Hobart mixer. The dry components were mixed for 5 minutes. Then, water was added to the mixing bowl at a constant rate to reach acceptable granulation results. The resulting wet granulation was manually pressed through a 16-mesh screen and dried at 50 Deg C. to an acceptable moisture level. Finally, the dry granulation was manually sized using a 16-mesh screen

Next, the push and the barrier layer granulations were compressed into bilayer arrangements. 85 mg of barrier layer granulation was compressed with 270 mg of push layer granulation using the rotary tablet press with 0.278″(7 mm) tooling.

Next, the osmotic module was assembled as follows: bilayer arrangements of push and barrier layers were inserted to a depth of 0.525 inches into the size 0, transparent HPMC capsule body.

Next, the assembled osmotic modules were coated with a semi-permeable wall. The wall forming composition comprised 90% cellulose acetate having a 39.8% acetyl content and 10% poloxamer 188. The wall-forming composition was dissolved in acetone. The wall-forming composition was sprayed onto and around the bilayered arrangements in a pan coater until approximately 60 mg of membrane was applied to each tablet.

Next, a 20 mil (0.51 mm)) exit passageway was drilled through the semi-permeable wall to connect the drug layer with the exterior of the dosage system. The residual solvent was removed by drying at 45° C. and 45% RH for 24 hours followed by drying at 45° C. and ambient humidity for additional 24 hours.

Next, a liquid drug layer composition was prepared as follows: 29.862 g of polysorbate 80 was weighed into the glass jar. Then, 15 mg of butylated hydroxytoluene was mixed with polysorbate 80 for 30 seconds. Finally, 0.123 g of paliperidone was added into solution, pre-mixed with a spatula for 30 seconds and then mixed on a stirring plate for 20 hours.

Next, the empty compartment of the osmotic module was filled with a liquid drug layer using syringe. Approximately 500 mg of the liquid drug layer was dispensed into each osmotic module.

Next, the empty compartment of the osmotic module was filled with liquid drug layer using syringe. Approximately 500 mg of the liquid drug layer was dispensed into each osmotic module.

### Example 7

### Pharmacokinetics of Paliperidone and Risperidone when Administered as Osmotic Modules and Oral Solutions in Healthy Volunteers

The study investigated the pharmacokinetics of single doses of paliperidone and risperidone following administration of oral solution and in a prototype controlled release formulation (osmotic modules). This was a single-center, open-label, randomized, four-treatment, four-sequence, four-period, crossover pilot study in healthy males and females to characterize the pharmacokinetics of paliperidone and risperidone when administered as osmotic modules and oral solutions. Sixteen subjects were to be dosed with paliperidone and risperidone to ensure that at least 12 subjects completed the study.

Each subject received 2 mg risperidone, and 2 mg paliperidone according to the following four treatments:
Treatment A—Osmotic module (2 mg risperidone, prepared according to Example 5)
Treatment B—Solution (2 mg risperidone) administered orally as a bolus
Treatment C—Osmotic module (2 mg paliperidone, prepared according to Example 6)
Treatment D—Solution (2 mg paliperidone) administered orally as a bolus

Subjects received both risperidone treatments before receiving the paliperidone treatments. Treatments were separated by a washout period of not less than 6 days and not more than 14 days. The washout period began 24 h after dosing. Sixteen subjects were enrolled in the study, and one subject withdrew 8 days after the second study period. During the osmotic module treatments, fifteen blood samples (7 mL each sample) were collected from each subject for measurement of risperidone and paliperidone (risperidone treatment), or paliperidone (paliperidone treatment) plasma concentrations. Samples were obtained at 0 (pre-dose), 1, 2, 4, 6, 9, 12, 15, 16, 18, 24, 36, 48, 72, and 96 hours post dose.

During the oral solution treatments, fifteen blood samples (7 mL each sample) were collected from each subject for measurement of risperidone and paliperidone (risperidone treatment), or paliperidone (paliperidone treatment) plasma concentrations. Samples were obtained at 0 (pre-dose), 0.5, 1, 1.5, 2.5, 4, 6, 9, 12, 18, 24, 36, 48, 72, and 96 hours post dose.

PK parameters AUCₜ, AUC_{inf}, Cₘₐₓ, Tₘₐₓ, and t_{1/2} were calculated for paliperidone for each treatment and subject. Risperidone and active moiety (risperidone+paliperidone) parameters were estimated for the two risperidone treatments.

Sixteen subjects completed risperidone treatments (Treatments A and B), and 15 subjects completed paliperidone treatments (Treatments C and D). Table 16 and 17 presents the summary of the mean pharmacokinetic parameters.

The osmotic module treatment resulted in a much lower Cₘₐₓ and provided later peaks (Tₘₐₓ) compared to the oral solution treatment of each drug.

The relative bioavailability (BA) of risperidone, paliperidone, and active moiety following risperidone osmotic module dosing relative to oral solution was 59.6%, 67.1%, and 65.6%, respectively. The BA of paliperidone osmotic module relative to the oral solution was 62.5%.

The drug-to-metabolite ratios were similar following administration of risperidone via osmotic module and oral solution, which suggests that the drug metabolism is similar between the two formulations.

The AUC and relative BA of the active moiety following risperidone are similar to the AUC and relative BA following paliperidone for both formulations.

TABLE 16
Pharmacokinetic Data following Risperidone Treatments (A and B; n = 16)

| | 2 mg | | 2 mg | | 2 mg | | 2 mg | | 2 mg | | 2 mg | |
|---|---|---|---|---|---|---|---|---|---|---|---|---|
| | Risperidone | | Risperidone | | Risperidone | | Risperidone | | Risperidone | | Risperidone | |
| | Osmotic Module | | Oral | | Osmotic Module | | Oral | | Osmotic Module | | Oral | |
| | Risperidone | | Risperidone | | Paliperidone | | Paliperidone | | Active Moiety | | Active Moiety | |
| Parameters | | | | | | | | | | | | |
| Cₘₐₓ (ng/mL) | 4.28 | (2.50) | 15.57 | (6.23) | 4.35 | (1.36) | 8.48 | (3.64) | 7.83 | (1.80)¹ | 21.80 | (5.46)¹ |
| Tₘₐₓ (h) | 6.63 | (2.34) | 0.99 | (0.23) | 15.20 | (6.02) | 6.63 | (5.44) | 8.13 | (2.50)¹ | 1.87 | (2.06)¹ |
| t_{1/2} (h) | 7.6 | (5.9)³ | 6.5 | (6.4)³ | 24.4 | (6.3) | 27.1 | (9.4)³ | — | | — | |
| AUCₜ(ng · h/mL) | 77.3 | (81.3) | 128.2 | (127.9) | 166.5 | (46.0) | 260.9 | (83.9) | 243.7 | (79.7)² | 389.1 | (142.6)² |
| AUC_{inf} | 79.6 | (83.4) | 136.1 | (145.2) | 182.4 | (47.7) | 283.7 | (83.8) | 262.0 | (87.6)² | 419.8 | (169.0)² |
| (ng · h/mL) | | | | | | | | | | | | |
| Relative | 59.6% | (18.8%) | N/A | | 67.1% | (15.6%) | N/A | | 65.6% | (16.7%) | N/A | |
| Bioavailability | | | | | | | | | | | | |
| Module/Oral | | | | | | | | | | | | |
| Dose Normalized | | | | | | | | | | | | |
| Parameters | | | | | | | | | | | | |
| Cₘₐₓ (ng/mL) | 2.14 | (1.25) | 7.8 | (3.15) | 2.2 | (0.68) | 4.24 | (1.82) | 3.92 | (0.9) | 10.9 | (2.73) |
| AUC_{inf} | 39.8 | (41.7) | 68.1 | (72.6) | 91.2 | (23.9) | 141.9 | (41.9) | 131.0 | (43.8) | 209.9 | (84.5) |
| (ng · h/mL) | | | | | | | | | | | | |

¹Cₘₐₓ and Tₘₐₓ values estimated from concentration profile of the sum of risperidone and paliperidone
²AUC values estimated by sum of AUC values of risperidone and paliperidone
³n = 15

TABLE 16
Pharmacokinetic Data following Paliperidone
Treatments (C and D; n = 15)

| | 2 mg Paliperidone | 2 mg Paliperidone |
|---|---|---|
| | Osmotic module | Oral solution |
| Parameters | | |
| Cₘₐₓ (ng/mL) | 6.39 (1.91) | 17.72 (4.47) |
| Tₘₐₓ (h) | 11.27 (3.4) | 1.31 (0.59) |
| t_{1/2} (h) | 27.5 (4.3) | 29.3 (9.9)¹ |
| AUCₜ (ng · h/mL) | 246.0 (75.4) | 399.6 (103.8) |
| AUC_{inf} (ng · h/mL) | 271.4 (81.8) | 439.4 (128.6) |
| Relative Bioavailability | 62.5% (11.3%) | N/A |
| Module/oral | | |
| Dose Normalized Parameters | | |
| Cₘₐₓ (ng/mL) | 3.2 (0.96) | 8.9 (2.24) |
| AUC_{inf} (ng · h/mL) | 135.7 (40.9) | 219.7 (64.3) |

¹n = 13

### Example 8

### Paliperidone Capsule Shaped Tablet, Trilayer 2 mg System

A dosage form adapted, designed and shaped as an osmotic drug delivery device was manufactured as follows: 120 g of paliperidone, 7325 g of polyethylene oxide with average molecular weight of 200,000, and 2000 g of sodium chloride, USP were added to a fluid bed granulator bowl. Next a binder solution was prepared by dissolving 400 g of hydroxypropylmethyl cellulose identified as 2910 having an average viscosity of 5 cps in 7,600 g of water. The dry materials were fluid bed granulated by spraying with 4,000 g of binder solution. Next, the wet granulation was dried in the granulator to an acceptable moisture content, and sized using by passing through a 7-mesh screen. Next, the granulation was transferred to a blender and mixed with 5 g of butylated hydroxytoluene as an antioxidant and lubricated with 50 g of stearic acid.

Next, a second drug compartment composition was prepared as follows: 280 g of paliperidone and 9165 g of polyethylene oxide with average molecular weight of 200,000 were added to a fluid bed granulator bowl. Next a binder solution was prepared by dissolving 400 g of hydroxypropylmethyl cellulose identified as 2910 having an average viscosity of 5 cps in 7,600 g of water. The dry materials were fluid bed granulated by spraying with 4,000 g of binder solution. Next, the wet granulation was dried in the granulator to an acceptable moisture content, and sized using by passing through a 7-mesh screen. Next, the granulation was transferred to a blender and mixed with 5 g of butylated hydroxytoluene as an antioxidant and lubricated with 50 g of stearic acid.

Next, a push composition was prepared as follows: first, a binder solution was prepared. 15.6 kg of polyvinylpyrrolidone identified as K29-32 having an average molecular weight of 40,000 was dissolved in 104.4 kg of water. Then, 24 kg of sodium chloride and 1.2 kg of ferric oxide were sized using a Quadro Comil with a 21-mesh screen. Then, the screened materials and 88.44 kg of Polyethylene oxide (approximately 7,000,000 molecular weight) were added to a fluid bed granulator bowl. The dry materials were fluidized and mixed while 46.2 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 15 g of butylated hydroxytoluene and lubricated with 294 g magnesium stearate.

Next, the paliperidone drug compositions for the first and the second compartments and the push composition were compressed into trilayer tablets. First, 50 mg of the paliperidone compartment one composition was added to the die cavity and pre-compressed, then 50 mg of the paliperidone compartment two composition was added to the die cavity and pre-compressed, then 100 mg of the push composition was added and the layers were pressed into a 3/16″ diameter longitudinal, deep concave, trilayer arrangement.

The trilayered arrangements were coated with a subcoat laminate. The wall forming composition comprised 70% hydroxypropyl cellulose identified as EF, having an average molecular weight of 80,000 and 30% of polyvinylpyrrolidone identified as K29-32 having an average molecular weight of 40,000. The wall-forming composition was dissolved in anhydrous ethyl alcohol, to make an 8% solids solution. The wall-forming composition was sprayed onto and around the bilayered arrangements in a pan coater until approximately 20 mg of laminate was applied to each tablet.

The trilayered arrangements were coated with a semi-permeable wall. The wall forming composition comprised 99% cellulose acetate having a 39.8% acetyl content and 1% polyethylene glycol comprising a 3.350 viscosity-average molecular weight. The wall-forming composition was dissolved in an acetone:water (95:5 wt:wt) co solvent to make a 5% solids solution. The wall-forming composition was sprayed onto and around the bilayered arrangements in a pan coater until approximately 40 mg of membrane was applied to each tablet.

Next, two 25 mil (0.6 mm) exit passageways were laser drilled through the semi-permeable wall to connect the drug layer with the exterior of the dosage system. The residual solvent was removed by drying for 144 hours as 45 Deg C. and 45% humidity. After drilling, the osmotic systems were dried for 4 hours at 45 Deg C. to remove excess moisture.

The dosage form produced by this manufacture was designed to deliver 2 mg of paliperidone in an ascending delivery pattern from two drug-containing cores. The first core contained 1.2% paliperidone, 73.25% polyethylene oxide possessing a 200,000 molecular weight, 20% sodium chloride, USP, 5% hydroxypropylmethyl cellulose having an average viscosity of 5 cps, 0.05% butylated hydroxytoluene, and 0.5% stearic acid. The second drug core contained 2.8% paliperidone, 91.65% polyethylene oxide possessing a 200,000 molecular weight, 5% hydroxypropylmethyl cellulose having an average viscosity of 5 cps, 0.05% butylated hydroxytoluene, and 0.5% stearic acid. The push composition comprised 73.7% polyethylene oxide comprising a 7,000,000 molecular weight, 20% sodium chloride, 5% polyvinylpyrrolidone possessing an average molecular weight of 40,000, 1% ferric oxide, 0.05% butylated hydroxytoluene, and 0.25% magnesium stearate. The semi permeable wall was comprised of 99% cellulose acetate of 39.8% acetyl content and 1% polyethylene glycol. The dosage form comprised two passageways, 25 mils (0.6 mm) on the center of the drug side.

### Example 9

### Paliperidone Capsule Shaped Tablet, Trilayer 2 mg System

A dosage form adapted, designed and shaped as an osmotic drug delivery device was manufactured as follows: 120 g of paliperidone, 7325 g of polyethylene oxide with average molecular weight of 200,000, and 2000 g of sodium chloride, USP were added to a fluid bed granulator bowl. Next a binder solution was prepared by dissolving 400 g of hydroxypropylmethyl cellulose identified as 2910 having an average viscosity of 5 cps in 7,600 g of water. The dry materials were fluid bed granulated by spraying with 4,000 g of binder solution. Next, the wet granulation was dried in the granulator to an acceptable moisture content, and sized using by passing through a 7-mesh screen. Next, the granulation was transferred to a blender and mixed with 5 g of butylated hydroxytoluene as an antioxidant and lubricated with 50 g of stearic acid.

Next, a second drug compartment composition was prepared as follows: 280 g of paliperidone and 9165 g of polyethylene oxide with an average molecular weight of 200,000 were added to a fluid bed granulator bowl. Next a binder solution was prepared by dissolving 400 g of hydroxypropylmethyl cellulose identified as 2910 having an average viscosity of 5 cps in 7,600 g of water. The dry materials were fluid bed granulated by spraying with 4,000 g of binder solution. Next, the wet granulation was dried in the granulator to an acceptable moisture content, and sized using by passing through a 7-mesh screen. Next, the granulation was transferred to a blender and mixed with 5 g of butylated hydroxytoluene as an antioxidant and lubricated with 50 g of stearic acid.

Next, a push composition was prepared as follows: first, a binder solution was prepared. 15.6 kg of polyvinylpyrrolidone identified as K29-32 having an average molecular weight of 40,000 was dissolved in 104.4 kg of water. Then, 24 kg of sodium chloride and 1.2 kg of ferric oxide were sized using a Quadro Comil with a 21-mesh screen. Then, the screened materials and 88.44 kg of Polyethylene oxide (approximately 7,000,000 molecular weight) were added to a fluid bed granulator bowl. The dry materials were fluidized and mixed while 46.2 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 15 g of butylated hydroxytoluene and lubricated with 294 g magnesium stearate.

Next, the paliperidone drug compositions for the first and the second compartments and the push composition were compressed into trilayer tablets. First, 50 mg of the paliperidone compartment one composition was added to the die cavity and pre-compressed, then 50 mg of the paliperidone compartment two composition was added to the die cavity and pre-compressed, then 100 mg of the push composition was added and the layers were pressed into a 3/16″ diameter longitudinal, deep concave, trilayer arrangement.

The trilayered arrangements were coated with a subcoat laminate. The wall forming composition comprised 70% hydroxypropyl cellulose identified as EF, having an average molecular weight of 80,000 and 30% of polyvinylpyrrolidone identified as K29-32 having an average molecular weight of 40,000. The wall-forming composition was dissolved in anhydrous ethyl alcohol, to make an 8% solids solution. The wall-forming composition was sprayed onto and around the bilayered arrangements in a pan coater until approximately 20 mg of laminate was applied to each tablet.

The trilayered arrangements were coated with a semi-permeable wall. The wall forming composition comprises 99% cellulose acetate having a 39.8% acetyl content and 1% polyethylene glycol comprising a 3.350 viscosity-average molecular weight. The wall-forming composition was dissolved in an acetone:water (95:5 wt:wt) co solvent to make a 5% solids solution. The wall-forming composition was sprayed onto and around the bilayered arrangements in a pan coater until approximately 20 mg of membrane was applied to each tablet.

Next, two 25 mil (0.6 mm) exit passageways were laser drilled through the semi-permeable wall to connect the drug layer with the exterior of the dosage system. The residual solvent was removed by drying for 144 hours as 45 Deg C. and 45% humidity. After drilling, the osmotic systems were dried for 4 hours at 45 Deg C. to remove excess moisture.

The dosage form produced by this manufacture was designed to deliver 2 mg of paliperidone in an ascending delivery pattern from two drug-containing cores. The first core contained 1.2% paliperidone, 73.25% polyethylene oxide possessing a 200,000 molecular weight, 20% sodium chloride, USP, 5% hydroxypropylmethyl cellulose having an average viscosity of 5 cps, 0.05% butylated hydroxytoluene, and 0.5% stearic acid. The second drug core contained 2.8% paliperidone, 91.65% polyethylene oxide possessing a 200,000 molecular weight, 5% hydroxypropylmethyl cellulose having an average viscosity of 5 cps, 0.05% butylated hydroxytoluene, and 0.5% stearic acid. The push composition comprised 73.7% polyethylene oxide comprising a 7,000,000 molecular weight, 20% sodium chloride, 5% polyvinylpyrrolidone possessing an average molecular weight of 40,000, 1% ferric oxide, 0.05% butylated hydroxytoluene, and 0.25% magnesium stearate. The semi permeable wall was comprised of 99% cellulose acetate of 39.8% acetyl content and 1% polyethylene glycol. The dosage form comprised two passageways, 25 mils (0.6 mm) on the center of the drug side.

### Example 10

### Evaluation of OROS® (Paliperidone) Pharmacokinetics and Pharmacodynamics

This study investigated the pharmacokinetics and pharmacodynamic effects (postural changes in blood pressure and heart rate) of 2 different formulations of OROS® (paliperidone) and compared with oral paliperidone solution and also evaluated the effect of food on the pharmacokinetics of SLOW OROS® (paliperidone).

This was a single-center, single-dose, open-label, randomized, 4-treatment, 4-sequence, 4-period, crossover study. Each subject received the following 4 treatments in a random manner (all doses refer to the total drug content in the formulation):
Treatment A—FAST OROS®(paliperidone), 4 mg (2×2 mg, prepared according to Example 9), Fasted
Treatment B—SLOW OROS® (paliperidone), 4 mg (2×2 mg, prepared according to Example 8), Fasted
Treatment C—SLOW OROS® (paliperidone), 4 mg (2×2 mg, prepared according to Example 8), with Food (FDA-standard high-fat breakfast; about half of the breakfast's ˜1000 calories were provided by fat)
Treatment D—Immediate-release (IR) Oral Solution paliperidone (in Tartaric acid solution at approximately pH 6.9-7.1), 2 mg, Fasted

Twenty-seven subjects received all 4 study treatments. The FAST OROS® (paliperidone) system was designed to release the dose over approximately 14 hours; the SLOW OROS® (paliperidone) system was designed to release the dose over approximately 24 hours. There was a 6- to 14-day washout period between treatments, which began 24 hours after dosing in each treatment period. During each treatment, blood samples were collected from each subject to determine plasma paliperidone concentrations. Samples were collected at:

FAST OROS® (paliperidone): 0 (pre-dose), 2, 4, 6, 8, 10, 11, 12, 13.5, 16, 18, 22, 24, 27, 30, 36, 42, 48, 58, 72, and 96 hours post dose for

SLOW OROS® (paliperidone): 0 (pre-dose), 2, 4, 6, 9, 12, 16, 18, 20, 22, 24, 27, 30, 33, 36, 42, 48, 58, 72, and 96 hours post dose

IR Oral Solution paliperidone treatment: 0 (pre-dose), 0.5, 1, 1.5, 2, 3, 4, 6, 9, 12, 18, 24, 36, 48, 58, 72, and 96 hours post dose.

Postural changes in blood pressure and heart rate were assessed with an automated blood pressure monitor during each treatment at 0 (pre-dose), 1, 2, 4, 8, 10, 12, 16, 20, 22, 24, 36, 48, 72, and 96 hours post dose. Two supine blood pressure and heart rate measurements were collected. At 2 and 3 minutes after standing from the supine position, blood pressure and heart rate were again measured. Dizziness and fainting symptoms after standing were assessed as described in Example 2.

PK parameters AUCₜ, AUC_{inf}, Cₘₐₓ, Tₘₐₓ, and t_{1/2} were calculated for paliperidone for each treatment and subject.

The percentage of subjects with >20 mm Hg drop in systolic blood pressure (SBP) at 3 minutes of standing or with symptoms of orthostatic hypotension (dizziness or faintness) was summarized for Days 1 and 2

FIG. 12 illustrates the mean paliperidone concentration profile. Pharmacokinetic parameters as well as ratios and 90% CIs are summarized in Table 18. The SLOW OROS® treatments (fasted and fed) resulted in a lower Cₘₐₓ and provided later peaks (Tₘₐₓ) compared with IR Oral Solution paliperidone. FAST OROS® treatment also resulted in a lower Cₘₐₓ and provide later peaks (Tₘₐₓ) compared with the IR Oral Solution paliperidone, but to a lesser degree than the SLOW OROS® treatments (fasted and fed).

Mean bioavailability estimated for FAST OROS® (paliperidone) and SLOW OROS® (paliperidone) in the fasted state was 52% and 34%, respectively, relative to IR Oral Solution. Mean bioavailability of SLOW OROS® in the fed state (40%) was higher than in the fasted state.

TABLE 18
Paliperidone Pharmacokinetic Parameters, Mean (CV), n = 27

| | | SLOW | | IR |
|---|---|---|---|---|
| | FAST OROS ® | OROS ® | SLOW OROS ® | Oral Solution |
| | Fasted | Fasted | Fed | Fasted |
| Parameter | 4 mg | 4 mg | 4 mg | 2 mg |
| Cₘₐₓ (ng/mL) | 12.2 (35%) | 6.7 (53%) | 8.2 (61%) | 19.4 (34%) |
| Tₘₐₓ (h) | 11.4 (18%) | 22.2 (17%) | 22.7 (16%) | 1.2 (47%) |
| t_{1/2} (h)^{a} | 25.95 (15%) | 28.17 (29%) | 25.81 (21%) | 26.98 (19%) |
| AUC₍₀₋₉₆₎ (ng · h/mL) | 372 (37%) | 243 (50%) | 285 (54%) | 371 (36%) |
| AUC_{inf} (ng · h/mL) | 403 (37%) | 272 (50%) | 314 (54%) | 397 (36%) |
| Bioavailability | 52 (31%) | 34 (31%) | 40 (48%) | Reference |
| (%) Range | 1-74 | 9-61 | 23-91 | |
| AUC_{inf} | 45% | 32% | 36% | Reference |
| Ratio (90% CI)^{b} | (36%-56%) | (26%-40%) | (29%-45%) | |
| Cₘₐₓ | NA | Reference | 115% | NA |
| Ratio (90% CI)^{b} | | | (93%-143%) | |
| AUC_{inf} | NA | Reference | 111% | NA |
| Ratio (90% CI)^{b} | | | (89%-139%) | |

Dose Normalized Cmax and AUC

| | | | | |
|---|---|---|---|---|
| Cₘₐₓ (ng/mL/mg) | 3.05 | 1.7 | 2.05 | 9.7 |
| AUC_{inf} | 101 | 68 | 78.5 | 199 |
| (ng · h/mL/mg) | | | | |

^{a}n = 25 for FAST OROS ® Fasted; n = 26 for SLOW OROS ® Fasted
^{b}Based on log-transformed analysis
NA = not applicable

Table 19 presents the percentage of subjects who experienced dizziness/faintness or had a drop in systolic blood pressure greater than 20 mm Hg at 2 minutes standing 0 to 24 hours after paliperidone administration. SLOW OROS® (paliperidone) treatments were associated with the lowest incidence of orthostatic hypotension among the treatments in this study. FAST OROS® (paliperidone) was associated with an incidence of orthostatic hypotension similar to that of the 2-mg dose of IR Oral Solution paliperidone.

TABLE 19
Number (%) of Subjects Who Felt Dizzy or Faint or
Had a Drop of >20 mmHg in Systolic Blood Pressure
After 2 Minutes Standing (0-24 Hours)

| | FAST | SLOW | SLOW | IR Oral |
|---|---|---|---|---|
| | OROS ® | OROS ® | OROS ® | Solution |
| | fasted | fasted | fed | fasted |
| | n = 28 | n = 30 | n = 31 | n = 29 |
| | 4 mg | 4 mg | 4 mg | 2 mg |
| Subjects with | 19 (68%) | 15 (50%) | 13 (42%) | 21 (72%) |
| dizziness/faintness or | | | | |
| >20 mmHg drop | | | | |
| Subjects with | 13 (46%) | 9 (30%) | 8 (26%) | 11 (38%) |
| >20 mmHg drop | | | | |

### Example 11

### Risperidone Capsule Shaped Tablet, Trilayer 2 mg System, SLOW

A dosage form adapted, designed and shaped as an osmotic drug delivery device was manufactured as follows: 130 g of risperidone, 7265 g of polyethylene oxide with average molecular weight of 200,000 (super fine particle size), and 2000 g of sodium chloride, USP were added to a fluid bed granulator bowl. Next a binder solution was prepared by dissolving 400 g of hydroxypropylmethyl cellulose identified as 2910 having an average viscosity of 5 cps in 7,600 g of water. The dry materials were fluid bed granulated by spraying with 4,000 g of binder solution. Next, the wet granulation was dried in the granulator to an acceptable moisture content, and sized using by passing through a 7-mesh screen. Next, the granulation was transferred to a blender and mixed with 5 g of butylated hydroxytoluene as an antioxidant and lubricated with 100 g of stearic acid.

Next, a second drug compartment composition was prepared as follows: 310 g of paliperidone and 9085 g of polyethylene oxide with average molecular weight of 200,000 (super fine particle size) were added to a fluid bed granulator bowl. Next, a binder solution was prepared by dissolving 400 g of hydroxypropylmethyl cellulose identified as 2910 having an average viscosity of 5 cps in 7,600 g of water. The dry materials were fluid bed granulated by spraying with 4,000 g of binder solution. Next, the wet granulation was dried in the granulator to an acceptable moisture content, and sized using by passing through a 7-mesh screen. Next, the granulation was transferred to a blender and mixed with 5 g of butylated hydroxytoluene as an antioxidant and lubricated with 100 g of stearic acid.

Next, a push composition was prepared as follows: first, a binder solution was prepared. 15.6 kg of polyvinylpyrrolidone identified as K29-32 having an average molecular weight of 40,000 was dissolved in 104.4 kg of water. Then, 24 kg of sodium chloride and 1.2 kg of ferric oxide were sized using a Quadro Comil with a 21-mesh screen. Then, the screened materials and 88.44 kg of Polyethylene oxide (approximately 7,000,000 molecular weight) were added to a fluid bed granulator bowl. The dry materials were fluidized and mixed while 46.2 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 15 g of butylated hydroxytoluene and lubricated with 294 g magnesium stearate.

Next, the paliperidone drug compositions for the first and the second compartments and the push composition were compressed into trilayer tablets. First, 50 mg of the paliperidone compartment one composition was added to the die cavity and pre-compressed, then 40 mg of the paliperidone compartment two composition was added to the die cavity and pre-compressed, then 110 mg of the push composition was added and the layers were pressed into a 3/16″ diameter longitudinal, deep concave, trilayer arrangement.

The trilayered arrangements were coated with a subcoat laminate. The wall forming composition comprised 70% hydroxypropyl cellulose identified as EF, having an average molecular weight of 80,000 and 30% of polyvinylpyrrolidone identified as K29-32 having an average molecular weight of 40,000. The wall-forming composition was dissolved in anhydrous ethyl alcohol, to make an 8% solids solution. The wall-forming composition was sprayed onto and around the bilayered arrangements in a pan coater until approximately 20 mg of laminate was applied to each tablet.

The trilayered arrangements were coated with a semi-permeable wall. The wall forming composition comprised 99% cellulose acetate having a 39.8% acetyl content and 1% polyethylene glycol comprising a 3.350 viscosity-average molecular weight. The wall-forming composition was dissolved in an acetone:water (95:5 wt:wt) co solvent to make a 5% solids solution. The wall-forming composition was sprayed onto and around the bilayered arrangements in a pan coater until approximately 40 mg of membrane was applied to each tablet.

Next, two 30 mil (0.76 mm) exit passageways were laser drilled through the semi-permeable wall to connect the drug layer with the exterior of the dosage system. The residual solvent was removed by drying for 144 hours as 45 Deg C. and 45% humidity. After drilling, the osmotic systems were dried for 4 hours at 45 Deg C. to remove excess moisture.

Next, the dried systems were overcoated with the drug-containing solution. The solution included risperidone, hydroxypropyl methylcellulose, and citric acid 1.31/97.43/1.26 wt/wt, respectively. The components were dissolved in water resulting in a solution with 7% solids. The drug overcoat composition was sprayed onto and around the dried systems in a pan coater until approximately 8 mg of overcoat was applied to each tablet. The tablets were dried in the coater after drug overcoating.

The dosage form produced by this manufacture was designed to deliver 2 mg of paliperidone in two modes: 0.1 mg as immediate release from the drug overcoat and 1.9 mg in an ascending delivery pattern from two drug-containing cores. The first core contained 1.3% paliperidone, 72.65% polyethylene oxide possessing a 200,000 molecular weight, 20% sodium chloride, USP, 5% hydroxypropylmethyl cellulose having an average viscosity of 5 cps, 0.05% butylated hydroxytoluene, and 1% stearic acid. The second drug core contained 3.1% paliperidone, 90.85% polyethylene oxide possessing a 200,000 molecular weight, 5% hydroxypropylmethyl cellulose having an average viscosity of 5 cps, 0.05% butylated hydroxytoluene, and 1% stearic acid. The push composition comprised 73.7% polyethylene oxide comprising a 7,000,000 molecular weight, 20% sodium chloride, 5% polyvinylpyrrolidone possessing an average molecular weight of 40,000, 1% ferric oxide, 0.05% butylated hydroxytoluene, and 0.25% magnesium stearate. The semi permeable wall was comprised of 99% cellulose acetate of 39.8% acetyl content and 1% polyethylene glycol. The dosage form comprised two passageways, 30 mils (0.76 mm) on the center of the drug side.

### Example 12

### Risperidone Capsule Shaped Tablet, Trilayer 2 mg System, FAST

A dosage form adapted, designed and shaped as an osmotic drug delivery device was manufactured as follows: 130 g of risperidone, 7265 g of polyethylene oxide with average molecular weight of 200,000 (super fine particle size), and 2000 g of sodium chloride, USP were added to a fluid bed granulator bowl. Next a binder solution was prepared by dissolving 400 g of hydroxypropylmethyl cellulose identified as 2910 having an average viscosity of 5 cps in 7,600 g of water. The dry materials were fluid bed granulated by spraying with 4,000 g of binder solution. Next, the wet granulation was dried in the granulator to an acceptable moisture content, and sized using by passing through a 7-mesh screen. Next, the granulation was transferred to a blender and mixed with 5 g of butylated hydroxytoluene as an antioxidant and lubricated with 100 g of stearic acid.

Next, a second drug compartment composition was prepared as follows: 310 g of paliperidone and 9085 g of polyethylene oxide with average molecular weight of 200,000 (super fine particle size) were added to a fluid bed granulator bowl. Next, a binder solution was prepared by dissolving 400 g of hydroxypropylmethyl cellulose identified as 2910 having an average viscosity of 5 cps in 7,600 g of water. The dry materials were fluid bed granulated by spraying with 4,000 g of binder solution. Next, the wet granulation was dried in the granulator to an acceptable moisture content, and sized using by passing through a 7-mesh screen. Next, the granulation was transferred to a blender and mixed with 5 g of butylated hydroxytoluene as an antioxidant and lubricated with 100 g of stearic acid.

Next, a push composition was prepared as follows: first, a binder solution was prepared. 15.6 kg of polyvinylpyrrolidone identified as K29-32 having an average molecular weight of 40,000 was dissolved in 104.4 kg of water. Then, 24 kg of sodium chloride and 1.2 kg of ferric oxide were sized using a Quadro Comil with a 21-mesh screen. Then, the screened materials and 88.44 kg of Polyethylene oxide (approximately 7,000,000 molecular weight) were added to a fluid bed granulator bowl. The dry materials were fluidized and mixed while 46.2 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 15 g of butylated hydroxytoluene and lubricated with 294 g magnesium stearate.

Next, the paliperidone drug compositions for the first and the second compartments and the push composition were compressed into trilayer tablets. First, 50 mg of the paliperidone compartment one composition was added to the die cavity and pre-compressed, then 40 mg of the paliperidone compartment two composition was added to the die cavity and pre-compressed, then 110 mg of the push composition was added and the layers were pressed into a 3/16″ diameter longitudinal, deep concave, trilayer arrangement.

The trilayered arrangements were coated with a subcoat laminate. The wall forming composition comprised 70% hydroxypropyl cellulose identified as EF, having an average molecular weight of 80,000 and 30% of polyvinylpyrrolidone identified as K29-32 having an average molecular weight of 40,000. The wall-forming composition was dissolved in anhydrous ethyl alcohol, to make an 8% solids solution. The wall-forming composition was sprayed onto and around the bilayered arrangements in a pan coater until approximately 20 mg of laminate was applied to each tablet.

The trilayered arrangements were coated with a semi-permeable wall. The wall forming composition comprised 99% cellulose acetate having a 39.8% acetyl content and 1% polyethylene glycol comprising a 3.350 viscosity-average molecular weight. The wall-forming composition was dissolved in an acetone:water (95:5 wt:wt) co solvent to make a 5% solids solution. The wall-forming composition was sprayed onto and around the bilayered arrangements in a pan coater until approximately 20 mg of membrane was applied to each tablet.

Next, two 30 mil (0.76 mm) exit passageways were laser drilled through the semi-permeable wall to connect the drug layer with the exterior of the dosage system. The residual solvent was removed by drying for 144 hours as 45 Deg C. and 45% humidity. After drilling, the osmotic systems were dried for 4 hours at 45 Deg C. to remove excess moisture.

Next, the dried systems were overcoated with the drug-containing solution. The solution included risperidone, hydroxypropyl methylcellulose, and citric acid 1.31/97.43/1.26 wt/wt, respectively. The components were dissolved in water resulting in a solution with 7% solids. The drug overcoat composition was sprayed onto and around the dried systems in a pan coater until approximately 8 mg of overcoat was applied to each tablet. The tablets were dried in the coater after drug overcoating.

The dosage form produced by this manufacture was designed to deliver 2 mg of paliperidone in two modes: 0.1 mg as immediate release from the drug overcoat and 1.9 mg in an ascending delivery pattern from two drug-containing cores. The first core contained 1.3% paliperidone, 72.65% polyethylene oxide possessing a 200,000 molecular weight, 20% sodium chloride, USP, 5% hydroxypropylmethyl cellulose having an average viscosity of 5 cps, 0.05% butylated hydroxytoluene, and 1% stearic acid. The second drug core contained 3.1% paliperidone, 90.85% polyethylene oxide possessing a 200,000 molecular weight, 5% hydroxypropylmethyl cellulose having an average viscosity of 5 cps, 0.05% butylated hydroxytoluene, and 1% stearic acid. The push composition comprised 73.7% polyethylene oxide comprising a 7,000,000 molecular weight, 20% sodium chloride, 5% polyvinylpyrrolidone possessing an average molecular weight of 40,000, 1% ferric oxide, 0.05% butylated hydroxytoluene, and 0.25% magnesium stearate. The semi permeable wall was comprised of 99% cellulose acetate of 39.8% acetyl content and 1% polyethylene glycol. The dosage form comprised two passageways, 30 mils (0.76 mm) on the center of the drug side.

### Example 13

### Evaluation of OROS® (Risperidone) and IR Risperidone Pharmacokinetics

This study investigated the pharmacokinetics of 2 different formulations of OROS® (risperidone) and compared with IR risperidone and also evaluated the effect of food on the pharmacokinetics of SLOW OROS® (risperidone).

This was a single-center, single-dose, open-label, randomized, 4-treatment, 4-sequence, 4-period, crossover study. Each subject received the following 4 treatments in a random manner (all doses refer to the total drug content in the formulation):
Treatment A: FAST OROS® (Risperidone), 2 mg, prepared as in Example 12, fasted.
Treatment B: SLOW OROS® (Risperidone), 2 mg, prepared as in Example 11, fasted.
Treatment C: SLOW OROS® (Risperidone), 2 mg, prepared as in Example 11, with food.
Treatment D: Immediate Release Risperidone, 2 mg (IR-2) fasted.

Thirty-two healthy males and females were enrolled and 24 subjects received all four study treatments. FAST OROS® and SLOW OROS® were designed to deliver the doses in approximately 14 hours and 24 hours, respectively. There was a 6- to 14-day washout period between treatments, which began 24 hours after dosing in each treatment period. During each treatment, blood samples were collected from each subject to determine plasma paliperidone concentrations. Samples were collected at:

FAST OROS® (Risperidone) 2 mg fasted: 0 (pre-dose), 1, 2, 4, 6, 8, 10, 11, 12, 13.5, 15, 18, 21, 24, 27, 30, 36, 42, 48, 58, 72, and 96 hours (h) after treatment initiation.

SLOW OROS® (Risperidone) 2 mg fasted the blood draw times were: 0 (pre-dose), 1, 2, 4, 6, 9, 12, 16, 18, 20, 22, 24, 27, 30, 33, 36, 42, 48, 58, 72, and 96 h after treatment initiation.

IR-2 dosing, the blood draw times were: 0 (pre-dose), 0.5, 1, 1.5, 3, 4, 6, 9, 12, 18, 24, 36, 48, 58, 72, and 96 hours h after treatment initiation.

PK parameters AUCₜ, AUC_{inf}, Cₘₐₓ, Tₘₐₓ, and t_{1/2} were calculated for paliperidone for each treatment and subject.

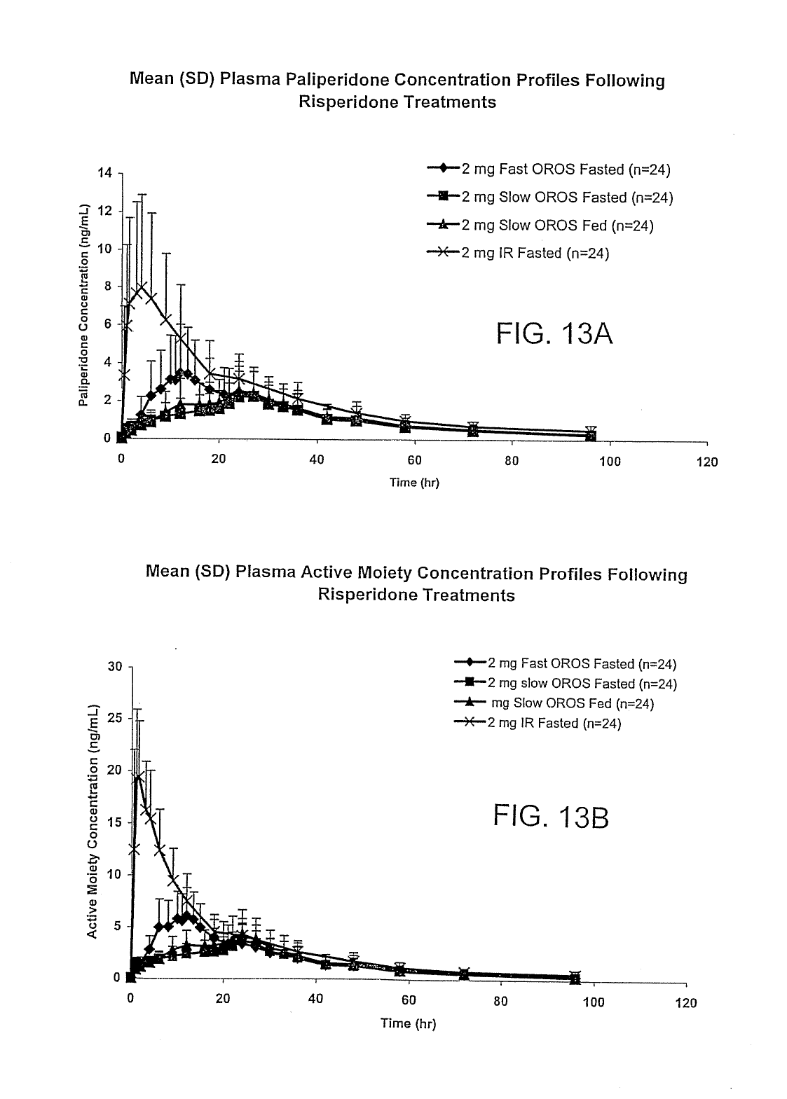

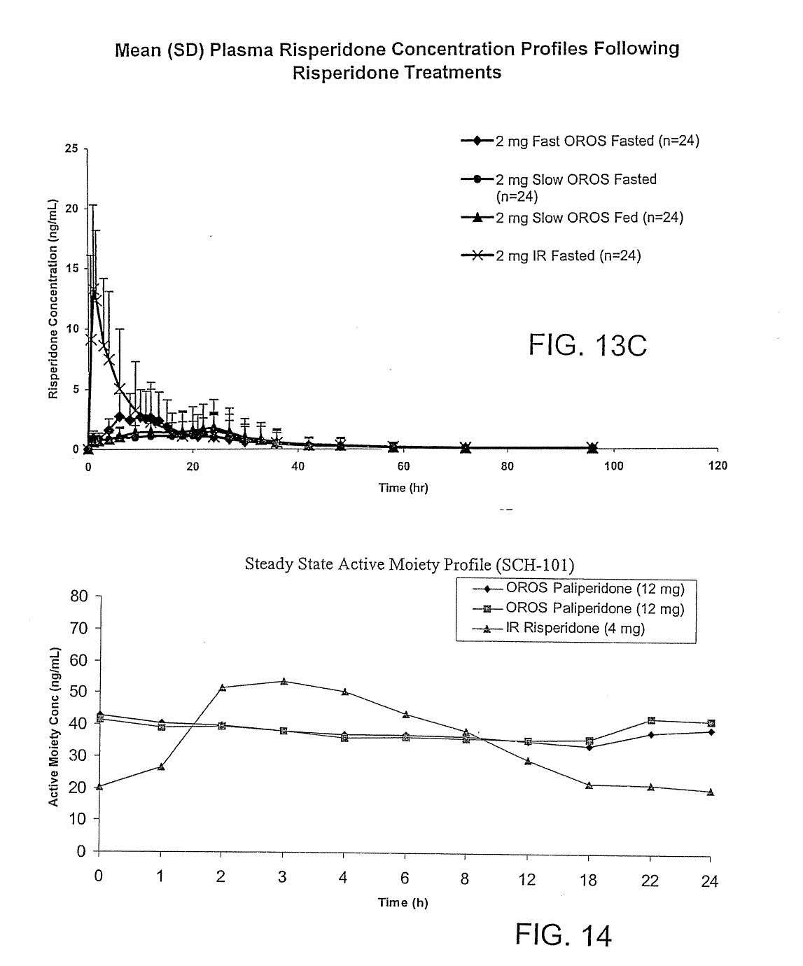

FIGS. 13A-C illustrates the mean risperidone, paliperidone and active moiety plasma concentration profiles following the risperidone treatments. Pharmacokinetic parameters as well as ratios and 90% CIs are summarized in Table 20.

The SLOW OROS® treatments (fasted and fed) resulted in a lower Cₘₐₓ and provided later peaks (Tₘₐₓ) compared with IR risperidone. FAST OROS® treatment also resulted in a lower Cₘₐₓ and provide later peaks (Tₘₐₓ) compared with the IR risperidone, but to a lesser degree than the SLOW OROS® treatments (fasted and fed). Mean half-life for risperidone and paliperidone values were similar among the four treatments.

Mean bioavailability estimated for FAST OROS® (risperidone) and SLOW OROS® (risperidone) in the fasted state for the three analytes was in the range of 52 to 55% and 41 to 42%, respectively, relative to IR-2 mg risperidone. Mean bioavailability of SLOW OROS® in the fed state (48 to 49%) was higher than in the fasted state (41 to 42%). The results of the ANOVA and 90% confidence intervals are also presented in Table 20.

TABLE 20
Pharmacokinetic Values Following Four Risperidone Treatments

| | | | | |
|---|---|---|---|---|
| | FAST OROS ® | SLOW OROS ® | SLOW OROS ® | |
| | (Risperidone) | (Risperidone) 2 mg | (Risperidone) 2 mg | Risperidone IR-2 mg |
| N = 24 | 2 mg (fasted) | (fasted) | (with food) | (fasted) |

Risperidone Concentration Mean (SD)

| | | | | | | | | |
|---|---|---|---|---|---|---|---|---|
| Cₘₐₓ (ng/mL) | 3.2 | (2.5) | 1.7 | (1.6) | 2.1 | (2.2) | 15.3 | (7.0) |
| Tₘₐₓ (h) | 9.1 | (2.8) | 15.2 | (10.7) | 14.6 | (8.1) | 1.1 | (0.5) |
| T_{1/2} (h) | 7.6 | (7.9) | 9.1 | (10.8)^{b} | 8.6 | (6.2)^{c} | 9.1 | (16.0) |
| AUCt (ng · h/mL) | 57.4 | (74.1) | 44.7 | (55.3) | 52.4 | (67.9) | 107.2 | (121.2) |
| AUC (0-96) (ng · h/mL) | 57.9 | (74.1) | 44.9 | (55.2) | 53.1 | (68.6) | 107.9 | (121.1) |
| AUCinf (ng · h/mL) | 60.2 | (78.6) | 47.4 | (59.7) | 54.9 | (70.2) | 113.4 | (134.4) |

| | | | | | | | |
|---|---|---|---|---|---|---|---|
| Bioavailability | 52.4 | (15.0) | 41.1 | (19.1) | 48.5 | (21.3) | Reference |

Dose Normalized Cmax and AUC

| | | | | | | | | |
|---|---|---|---|---|---|---|---|---|
| Cₘₐₓ (ng/mL/mg) | 1.6 | (1.25) | 0.85 | (0.8) | 1.05 | (1.1) | 7.7 | (3.5) |
| AUC_{inf} (ng · h/mL/mg) | 30.1 | (39.3) | 23.7 | (29.9) | 27.5 | (35.1) | 56.7 | (67.2) |
| ANOVA | | | | | | | | |

| | | | | | | | |
|---|---|---|---|---|---|---|---|
| AUC_{inf} (vs IR-2) | 50.2% | (43.9, 57.3) | 37.9% | (33.2, 43.4) | 45.4% | (39.6, 52.0) | Reference |
| Ratio (90% CI)^{a} | | | | | | | |

| | | | | | |
|---|---|---|---|---|---|
| Cₘₐₓ (vs SLOW | NA | Reference | 119.1% | (99.2, 143.0) | NA |
| fasted) Ratio^{a} | | | | | |
| (90% CI) | | | | | |
| AUC_{inf} vs SLOW | NA | Reference | 119.6% | (104.5, 136.9) | NA |
| fasted) Ratio^{a} | | | | | |
| (90% CI) | | | | | |

Paliperidone Concentration Mean (SD)

| | | | | | | | | |
|---|---|---|---|---|---|---|---|---|
| Cₘₐₓ (ng/mL) | 3.8 | (2.8) | 2.3 | (1.4) | 2.7 | (1.6) | 8.6 | (4.8) |
| Tₘₐₓ (h) | 15.1 | (6.1) | 26.4 | (4.3) | 24.6 | (5.8) | 5.6 | (5.9) |
| T_{1/2} (h) | 27.4 | (5.3)^{d} | 26.1 | (6.2) | 25.7 | (6.4) | 28.2 | (6.8)^{b} |
| AUCt (ng · h/mL) | 118.0 | (65.8) | 88.8 | (44.1) | 100.7 | (56.4) | 212.7 | (95.9) |
| AUC(0-96) (ng · h/mL) | 118.3 | (65.8) | 88.9 | (44.1) | 101.3 | (55.8) | 212.7 | (95.9) |
| AUCinf (ng · h/mL) | 130.3 | (68.3) | 98.7 | (46.7) | 112.8 | (62.2) | 232.8 | (99.5) |

| | | | | | | | |
|---|---|---|---|---|---|---|---|
| Bioavailability | 55.1 | (12.7) | 42.4 | (10.6) | 48.0 | (19.2) | Reference |

Dose Normalized Cmax and AUC

| | | | | | | | | |
|---|---|---|---|---|---|---|---|---|
| Cₘₐₓ (ng/mL/mg) | 1.9 | (1.4) | 1.2 | (0.7) | 1.35 | (0.8) | 4.3 | (2.4) |
| AUC_{inf} (ng · h/mL/mg) | 65.2 | (34.2) | 49.4 | (23.4) | 56.4 | (31.1) | 116.4 | (49.8) |
| ANOVA | | | | | | | | |

| | | | | | | | |
|---|---|---|---|---|---|---|---|
| AUC_{inf} (vs IR-2) | 54.3% | (48.8, 60.4) | 41.2% | (37.1, 45.9) | 45.8% | (41.2, 51.1) | Reference |
| Ratio (90% CI)^{a} | | | | | | | |

| | | | | | |
|---|---|---|---|---|---|
| Cₘₐₓ (vs SLOW | NA | Reference | 119.5% | (100.5, 142.0) | NA |
| fasted) Ratio | | | | | |
| (90% CI)^{a} | | | | | |
| AUC_{inf} vs SLOW | NA | Reference | 111.2% | (99.9, 123.9) | NA |
| fasted) Ratio^{a} | | | | | |
| (90% CI) | | | | | |

Active Moiety Concentration^{e} Mean (SD)

| | | | | | | | | |
|---|---|---|---|---|---|---|---|---|
| Cₘₐₓ (ng/mL) | 6.6 | (3.1) | 3.8 | (1.8) | 4.6 | (2.2) | 22.0 | (6.2) |
| Tₘₐₓ (h) | 11.0 | (2.1) | 22.1 | (5.7) | 20.9 | (6.3) | 1.1 | (0.5) |
| AUCt (ng · h/mL) | 175.4 | (82.8) | 133.5 | (59.9) | 153.1 | (76.0) | 319.9 | (109.3) |
| AUC(0-96) (ng · h/mL) | 176.2 | (82.6) | 133.8 | (59.8) | 154.4 | (76.5) | 320.6 | (109.1) |
| AUCinf (ng · h/mL) | 190.5 | (88.3) | 146.1 | (66.9) | 167.7 | (82.7) | 346.2 | (126.1) |

| | | | | | | | |
|---|---|---|---|---|---|---|---|
| Bioavailability | 54.5 | (12.8) | 41.8 | (11.1) | 48.4 | (19.4) | Reference |

Dose Normalized Cmax and AUC

| | | | | | | | | |
|---|---|---|---|---|---|---|---|---|
| Cₘₐₓ (ng/mL/mg) | 3.3 | (1.6) | 1.9 | (0.9) | 2.3 | (1.1) | 11 | (3.1) |
| AUC_{inf} (ng · h/mL/mg) | 95.3 | (44.2) | 73.1 | (33.5) | 83.9 | (41.4) | 173.1 | (63.1) |
| ANOVA | | | | | | | | |

| | | | | | | | |
|---|---|---|---|---|---|---|---|
| AUC_{inf} (vs IR-2) | 53.4% | (47.9, 59.5) | 40.7% | (36.5, 45.3) | 46.3% | (41.5, 51.7) | Reference |
| Ratio (90% CI)^{a} | | | | | | | |

| | | | | | |
|---|---|---|---|---|---|
| Cₘₐₓ (vs SLOW | NA | Reference | 121.4% | (104.0, 141.7) | NA |
| fasted) Ratio^{a} | | | | | |
| (90% CI) | | | | | |
| AUC_{inf} vs SLOW | NA | Reference | 114.0% | (102.1, 127.2) | NA |
| fasted) Ratio^{a} | | | | | |
| (90% CI) | | | | | |

^{a}Used a log transformation and ANOVA
^{b}n = 22;
^{c}n = 21;
^{d}n = 23
^{e}Cmax and Tmax values estimated from the concentration profile of the sum of risperidone and paliperidone.
AUC values estimated by sum of AUC values of risperidone and paliperidone.

### Example 14

### A Randomized, Double-Blind, Placebo- and Active-Controlled, Parallel-Group, Phase 1 Study to Compare the Tolerability of OROS Paliperidone (Extended-Release) with Immediate-Release (IR) Risperidone in Subjects with Schizophrenia

The primary objective of the study was a noninferiority comparison of the orthostatic tolerability of a dose of 12 mg ER OROS paliperidone (dosed as 6×2 mg tablets prepared as in Example 8, SLOW OROS) with the current recommended initial titration dose (2 mg) of risperidone in patients with schizophrenia. Secondary objectives were: (1) to compare the tolerability and safety of a clinically equivalent fixed dose of ER OROS paliperidone with the currently recommended dose of risperidone; (2) to compare the early tolerability of the 2 treatments with placebo.

This was a randomized, double-blind, placebo- and active-controlled, parallel group, Phase 1, study conducted at 9 study centers. The study consisted of a 1-week, open-label, placebo washout period (Days −7 to −1) and a 6-day double-blind treatment period during which subjects received 1 of 3 treatments:
placebo on Day 1 and ER OROS paliperidone (12 mg, 6×2 mg) on Days 2 to 6 (PLAC/PAL OROS group);
ER OROS paliperidone (12 mg, 6×2 mg) on Days 1 to 6 (PAL OROS group)
IR risperidone 2 mg on Day 1 and 4 mg on Days 2 to 6 (RIS IR group).
   120 subjects were planned to be enrolled in the study.

The study included men and women, 18 to 65 years-of age, with a diagnosis of schizophrenia based on DSM-IV criteria including paranoid type (295.30), disorganized type (295.10), catatonic type (295.20), undifferentiated type (295.90), or residual type (295.60). Subjects were to have stable schizophrenia defined as absence of acute exacerbation for at least 6 months before screening and to be treated with oral IR risperidone for at least 1 month at study entry.

Plasma concentrations of risperidone, paliperidone, and active moiety (i.e., the sum of paliperidone and risperidone plasma concentrations) were measured for pharmacokinetic and pharmacokinetic/pharmacodynamic evaluations.

Systolic (SBP) and diastolic (DBP) blood pressure and heart rate (HR) measurements were performed after 10 minutes in the supine position and 1, 3, and 5 minutes after standing during the Standing Monitored Test (SMT). Orthostatic intolerance was also assessed on the basis of symptoms (e.g., feeling dizzy or faint) and the results recorded on an Orthostatic Intolerance Visual Analog Scale (VAS). Serum concentrations of prolactin were determined. Psychiatric Evaluations: Two psychiatric rating scales, the Positive and Negative Syndrome Scale (PANSS) and the Clinical Global Impression (CGI) Scale, were administered to monitor for any possible deterioration in the subject's condition.

Safety assessments included reports of adverse events, the Extrapyramidal Symptom Rating Scale (ESRS), Sedation Visual Analog Scale (VAS), Leeds Sleep Evaluation Questionnaire (LSEQ), clinical laboratory tests, vital sign measurements, physical examinations, and electrocardiogram (ECG) findings.

Descriptive statistics for the plasma concentrations and PK parameters of paliperidone for both ER OROS paliperidone treatments and of risperidone, paliperidone and active moiety for the RIS IR treatment were summarized.

The primary variable i.e., the mean of 2 hour and 22 hour orthostatic SBP changes from baseline on Day 1, was analyzed using a linear regression model with treatment as a fixed factor and age as a continuous, linear covariate. A 95% CI for the difference in means between 12 mg ER OROS paliperidone and 2 mg IR risperidone was constructed using the estimates of LS means and intersubject variance from the regression model. The primary analysis was performed in the per-protocol population and repeated in the intent-to-treat population. Descriptive statistics were calculated for prolactin levels.

The incidence of treatment-emergent adverse events was summarized. Descriptive statistics were provided for the other safety parameters. These summaries were performed in the intent-to-treat population, unless specified otherwise.

The study population comprised 83 men and 30 women (113 subjects in total), and was randomly assigned to receive either PLAC/PAL OROS (n=37), PAL OROS (n=38), or RIS IR (n=38).

On Day 1, the mean peak plasma concentration for the active moiety (RIS IR treatment) was reached at 2.7 hours and for paliperidone in the ER OROS paliperidone treatment at 21.8 hours, which is close to the predicted values of 2 and 22 hours, respectively. The obtained mean Cmax in both treatments was 19.4 and 23.1 ng/mL respectively. On Day 6, the Tmax in both ER OROS paliperidone treatments was between 20 and 26 hours and steady state was reached after 4 to 5 days. FIG. 14 shows the mean steady state active moiety concentration profile.

The peak/trough variation of ER OROS paliperidone was much lower compared to risperidone IR reflected in a lower fluctuation index for the ER OROS paliperidone treatment versus the RIS IR treatment, 38% and 125% respectively.

On Day 1, mean orthostatic systolic blood pressure changes were:
−1.16 mmHg in PAL OROS-treated subjects,
−0.16 mmHg in RIS-IR treated subjects, and
−0.15 mmHg in placebo treated subjects.

The upper and lower limits of the 95% confidence interval for the difference in means between 12 mg ER OROS paliperidone and 2 mg IR risperidone were −4.07 and 2.02 mmHg indicating that 12 mg ER OROS paliperidone was noninferior to 2 mg IR risperidone with respect to initial orthostatic tolerability (i.e., the lower limit of −4.07 was greater than the predefined limit of −10 mmHg). The number of subjects with orthostatic hypotension or reflex tachycardia within 3 minutes after standing was lower in ER OROS paliperidone treated subjects (55%) than in IR risperidone treated subjects (79%). The peak/trough variation of prolactin in the ER OROS paliperidone treatment groups was much lower than the peak/trough variation of prolactin in the IR risperidone treatment group. For subjects in the PAL OROS group, the mean Tmax of prolactin was 6.5 hours while the mean Tmax of paliperidone was 21.8 hours.

The most commonly reported adverse events in the ER OROS paliperidone groups were extrapyramidal disorder (12%), insomnia (8%), hyperkinesia and headache (both 5%). The most commonly reported adverse events in the IR risperidone group were insomnia (18%), anxiety (11%), extrapyramidal disorder and tachycardia (both 8%), and hyperkinesia (5%). In the PLAC/PAL OROS group, all 4 reports of extrapyramidal disorder and 1 report of hyperkinesia had their initial onset on Day 1 during placebo treatment. The majority of adverse events was of mild intensity and resolved spontaneously.

Eleven (16%) of 69 subjects treated with ER OROS paliperidone and 6 (16%) of 38 subjects in the RIS IR treatment group experienced EPS-related adverse events (extrapyramidal disorder, hyperkinesia, ataxia, dystonia) after receiving at least 1 dose of ER OROS paliperidone or IR risperidone. Consistent with the reports of EPS-related adverse events, ER OROS paliperidone-treated subjects had a mean decrease (improvement) in total ESRS of 0.19 while IR risperidone-treated subjects had a mean increase (worsening) in total ESRS of 0.31. No serious adverse events occurred in either paliperidone treatment group. Five subjects discontinued double-blind treatment due to adverse events including 2 subjects in the PAL OROS group (psychosis, hypertension), 1 subject in the PLAC/PAL OROS group (hyperkinesia), and 2 subjects in the RIS IR group (psychosis, rhinitis). There were no clinically noteworthy changes from baseline in sleep quality parameters (LSEQ), level of alertness (sedation VAS and Questionnaire), clinical laboratory analytes, ECG, or body weight measurements.

In this population with stable schizophrenia patients, after 6 days of treatment, the PANSS and CGI scores for both groups with OROS paliperidone were combined and compared with IR risperidone group. Mean change in total PANSS on Day 7 was comparable between the OROS paliperidone and IR risperidone group. The CGI changes at Day 7 were in general comparable between OROS paliperidone and IR risperidone group.

In conclusion the results of this study indicate that 12 mg ER OROS paliperidone is noninferior to 2 mg IR risperidone with respect to initial orthostatic tolerability in subjects with schizophrenia. The safety profile of 12 mg ER OROS paliperidone once daily for 5 to 6 days was similar to that of 2 mg IR risperidone on Day 1 followed by 4 mg IR risperidone on Days 2 to 6, and was consistent with its expected safety profile based on its pharmacologic activity as a serotonin-dopamine antagonist, with no unexpected or unusual safety issues. The early tolerability as observed on Day 1 was comparable between the 3 treatment groups (12 mg ER OROS paliperidone, 2 mg IR risperidone, and placebo). During administration of 12 mg ER OROS paliperidone for 5 or 6 days in subjects with schizophrenia, Tmax of paliperidone was between 20 and 26 hours after dosing on Day 6 and steady state was reached within 4 to 5 days. The fluctuation index of ER OROS paliperidone was 69% lower than that of IR risperidone.

### Example 15

### Risperidone Capsule Shaped Tablet, Trilayer 1 mg System

A dosage form adapted, designed and shaped as an osmotic drug delivery device was manufactured as follows: 188.0 g of poly(ethylene oxide) possessing a 200,000 molecular weight, and 10.0 g of hydroxpropylmethylcellulose comprising a 11,200 molecular weight were added to a Kitchenaid planetary mixing bowl. Next, the dry materials were mixed for approximately 1 minute. Then, 100 ml of denatured anhydrous alcohol was slowly added to the blended materials with continuous mixing for approximately 2 minutes. Next, the wet granulation was allowed to dry at room temperature over night, and then passed through a 10-mesh screen. Finally, 2.0 g of stearic acid was mixed into the granulation for 3 minutes.

Next, a drug granulation was prepared as follows: 6.6 g of risperidone, 181.4 g of poly(ethylene oxide) possessing a 200,000 molecular weight, and 10.0 g of hydroxpropylmethylcellulose comprising a 11,200 molecular weight were added to a Kitchenaid planetary mixing bowl. Next, the dry materials were mixed for approximately 1 minute. Then, 100 ml of denatured anhydrous alcohol was slowly added to the blended materials with continuous mixing for approximately 2 minutes. Next, the wet granulation was allowed to dry at room temperature over night, and then passed through a 10-mesh screen. Finally, 2.0 g of stearic acid was mixed into the granulation for 3 minutes.

Next, a push composition was prepared as follows: first, a binder solution was prepared. 7.80 kg of poly(vinylpyrrolidone) identified as K29-32 having an average molecular weight of 40,000 was dissolved in 52.2 kg of water. 26,000 g of sodium chloride and 1300 g of ferric oxide was sized using a Quadro Comil with a 21-mesh screen. Then, all the screened materials, and 95,810 g of pharmaceutically acceptable poly(ethylene oxide) comprising a 7,000,000 molecular weight were added to a Glatt Fluid Bed Granulator's bowl. The bowl was attached to the granulator and the granulation process was initiated for effecting granulation. Next, the dry powders were air suspended and mixed. Then, the binder solution was sprayed from 3 nozzles onto the powder. The granulating conditions were monitored during the process as follows: total solution spray rate of 700 g/min; inlet temperature 45 Deg C.; and process airflow of 500-5000 m³/hr. While spraying the binder solution, the filter bags were shaken for 10 seconds every 30 seconds to unglue any possible powder deposits. At the end of the solution spraying, 50,000 g of the coated granulated particles were continued with the drying process. The machine was turned off, and the coated granules were removed from the granulator. The coated granules were sized using a Fluid Air mill with a 6 mesh screen. The granulation was transferred to a Tote Tumbler, mixed with 65 g of butylated hydroxytoluene and lubricated with 325 g stearic acid.

Next, the placebo composition, the drug composition and the push composition were compressed into trilayer tablets on the Carver Tablet Press into a 3/16″ (0.476 cm) diameter deep concave longitudinal layered arrangement. First, 70 mg of the placebo composition was added to the die cavity and pre-compressed, then 30 mg of the drug composition was added to the cavity and pre-compressed. Finally, 130 mg of the push composition was added and the layers were pressed under a pressure head of approximately ¼ a metric ton.

The trilayered arrangements were coated with a subcoat layer. The wall forming composition comprised 70% hydroxypropyl cellulose having an average molecular weight of 60,000 and 30% poly(vinylpyrrolidone) identified as K29-32 having an average molecular weight of 40,000. The wall-forming composition was dissolved in ethanol to make a 6% solids solution. The wall-forming composition was sprayed onto and around the bilayers in a 12″ Vector HiCoater.

The subcoated arrangements were coated with a semi-permeable wall. The wall forming composition comprised 99% cellulose acetate having a 39.8% acetyl content and 1% polyethylene glycol comprising a 3350 viscosity-average molecular weight. The wall-forming composition was dissolved in an acetone:water (95:5 wt:wt) cosolvent to make a 5% solids solution. The wall-forming composition was sprayed onto and around the subcoated arrangements in a 12″ Vector HiCoater.

Next, one 30 mil (0.762 mm) exit passageway was drilled through the semi-permeable wall to connect the drug layer with the exterior of the dosage system. The residual solvent was removed by drying for 60 hours at 45 Deg C. and 45% humidity. Next, the osmotic systems were dried for 4 hours at 45 Deg C. to remove excess moisture.

The dosage form produced by this manufacture provided 94.0% poly(ethylene oxide) possessing a 200,000 molecular weight, 5.0% hydroxpropylmethylcellulose of approximately 11,200 molecular weight, and 1.0% stearic acid. The drug composition comprised 3.3% risperidone, 90.7% poly(ethylene oxide) possessing a 200,000 molecular weight, 5.0% hydroxpropylmethylcellulose that comprised a 11,200 molecular weight, and 1.0% stearic acid. The push composition comprised 73.7% poly(ethylene oxide) comprising a 7,000,000 molecular weight, 20% sodium chloride, 5% poly(vinylpyrrolidone) identified as K29-32 having an average molecular weight of 40,000, 1.0% ferric oxide, 0.05% butylated hydroxytoluene, and 0.25% stearic acid. The subcoat wall comprised 70% hydroxypropyl cellulose having an average molecular weight of 60,000 and 30% poly(vinylpyrrolidone) identified as K29-32 having an average molecular weight of 40,000. The semipermeable wall comprised 99 wt % cellulose acetate comprising a 39.8% acetyl content and 1% polyethylene glycol comprising a 3,350 viscosity-average molecular weight. The dosage form comprised one passageway, 30 mils (0.762 mm), and it had a maximum risperidone release rate of 0.108 mg/hr.

### Example 16

### Risperidone Capsule Shaped Tablet, Trilayer 2 mg System

A dosage form adapted, designed and shaped as an osmotic drug delivery device was manufactured as follows: first, a binder solution was prepared. 600 g of hydroxpropylmethylcellulose comprising a 11,200 molecular weight was dissolved in 5,400 g of water. 2,000 g of sodium chloride was screened with a 21-mesh screen. For the first drug granulation, 7,340 g of poly(ethylene oxide) possessing a 200,000 molecular weight, 2,000 g of screened sodium chloride and 300 g of hydroxpropylmethylcellulose comprising a 11,200 molecular weight were added to a Freund Fluid Bed Granulator's bowl. The bowl was attached to the granulator and the granulation process was initiated for effecting granulation. Next, the dry powders were air suspended and mixed. Then, the binder solution was sprayed from two nozzles onto the powder. The granulating conditions were monitored during the process as follows: total solution spray rate of 80 ml/min, an exhaust temperature of approximately 22 C and airflow of 200-300 cfm. While spraying the binder solution, the filter bags were shaken for 10 seconds after every 30 second spray cycle to unglue any possible powder deposits. 250 g of binder solution was sprayed onto the in materials the granulator and the granulation process was paused. 136.5 g of risperidone was then added into the granulator bowl. The granulation process was then continued using the same processing conditions. At the end of the solution spraying, 2000 g of the coated granulated particles were continued with the drying process. The machine was turned off, and the coated granules were removed from the granulator. The coated granules were passed through a 7 mesh screen. Finally, the dried and screened granulation were transferred to an appropriate container, mixed and lubricated with 99.4 g of stearic acid and 5.0 g of butylated hydroxytoluene for 10 minutes.

For the second drug granulation, 9,085 g of poly(ethylene oxide) possessing a 200,000 molecular weight, and 300 g of hydroxpropylmethylcellulose comprising a 11,200 molecular weight were added to a Freund Fluid Bed Granulator's bowl. The bowl was attached to the granulator and the granulation process was initiated for effecting granulation. Next, the dry powders were air suspended and mixed. Then, the binder solution was sprayed from two nozzles onto the powder. The granulating conditions were monitored during the process as follows: total solution spray rate of 80 ml/min, an exhaust temperature of approximately 23 C and airflow of 200-300 cfm.

While spraying the binder solution, the filter bags were shaken for 10 seconds after every 30 second spray cycle to unglue any possible powder deposits. 250 g of binder solution was sprayed onto the materials in the granulator and the granulation process was paused. 325.5 g of risperidone was then added into the granulator bowl. The granulation process was then continued using the same processing conditions. At the end of the solution spraying, 2000 g, the coated granulated particles were continued with the drying process. The machine was turned off, and the coated granules were removed from the granulator. The coated granules were passed through a 7 mesh screen. Finally, the dried and screened granulation were transferred to an appropriate container, mixed and lubricated with 94.1 g of stearic acid and 4.7 g of butylated hydroxytoluene for 10 minutes.

Next, a push composition was prepared as follows: first, a binder solution was prepared. 7.80 kg of poly(vinylpyrrolidone) identified as K29-32 having an average molecular weight of 40,000 was dissolved in 52.2 kg of water. 26,000 g of sodium chloride and 1300 g of ferric oxide was sized using a Quadro Comil with a 21-mesh screen. Then, all the screened materials, and 95,810 g of pharmaceutically acceptable poly(ethylene oxide) comprising a 7,000,000 molecular weight were added to a Glatt Fluid Bed Granulator's bowl. The bowl was attached to the granulator and the granulation process was initiated for effecting granulation. Next, the dry powders were air suspended and mixed. Then, the binder solution was sprayed from 3 nozzles onto the powder. The granulating conditions were monitored during the process as follows: total solution spray rate of 700 g/min; inlet temperature 45 Deg C.; and process airflow of 500-5000 m³/hr.

While spraying the binder solution, the filter bags were shaken for 10 seconds every 30 seconds to unglue any possible powder deposits. At the end of the solution spraying, 50,000 g of the coated granulated particles were continued with the drying process. The machine was turned off, and the coated granules were removed from the granulator. The coated granules were sized using a Fluid Air mill with a 6 mesh screen. The granulation was transferred to a Tote Tumbler, mixed with 65 g of butylated hydroxytoluene and lubricated with 325 g stearic acid.

Next, the two drug composition and the push composition were compressed into trilayer tablets on the Korsch Multilayer Tablet Press into a 3/16″ (0.476 cm) diameter deep concave longitudinal layered arrangement. First, 50 mg of the first drug composition was added to the die cavity and pre-compressed using a 100N force, then 40 mg of the second drug composition was added to the cavity and pre-compressed using a 100N force. Finally, 110 mg of the push composition was added and the layers were pressed under a pressure head of approximately 2000N.

The trilayered arrangements were coated with a subcoat layer. The wall forming composition comprised 70% hydroxypropyl cellulose having an average molecular weight of 60,000 and 30% poly(vinylpyrrolidone) identified as K29-32 having an average molecular weight of 40,000. The wall-forming composition was dissolved in ethanol to make a 8% solids solution. The wall-forming composition was sprayed onto and around the bilayers in a 24″ Vector HiCoater.

The subcoated arrangements were coated with a semi-permeable wall. The wall forming composition comprised 99% cellulose acetate having a 39.8% acetyl content and 1% polyethylene glycol that comprised a 3350 viscosity-average molecular weight. The wall-forming composition was dissolved in an acetone:water (95:5 wt:wt) cosolvent to make a 5% solids solution. The wall-forming composition was sprayed onto and around the subcoated arrangements in a 24″ Vector HiCoater.

Next, two 30 mil (0.762 mm) exit passageway were drilled through the semi-permeable wall to connect the drug layer with the exterior of the dosage system. The residual solvent was removed by drying for 72 hours at 45 Deg C. and 45% humidity. Next, the osmotic systems were dried for 4 hours at 45 Deg C. to remove excess moisture.

The dosage form produced by this manufacture provided a first drug composition that comprised 1.3% risperidone, 72.8% poly(ethylene oxide) possessing a 200,000 molecular weight, 19.9% sodium chloride, 5.0% hydroxpropylmethylcellulose comprising a 11,200 molecular weight, 1.0% stearic acid, and 0.05% butylated hydroxytoluene. The second drug composition comprised 3.1% risperidone, 90.85% poly(ethylene oxide) possessing a 200,000 molecular weight, 5.0% hydroxpropylmethylcellulose comprising a 11,200 molecular weight, 1.0% stearic acid and 0.05% butylated hydroxytoluene. The push composition comprised 73.7% poly(ethylene oxide) comprising a 7,000,000 molecular weight, 20% sodium chloride, 5% poly(vinylpyrrolidone) identified as K29-32 having an average molecular weight of 40,000, 1.0% ferric oxide, 0.05% butylated hydroxytoluene, and 0.25% stearic acid. The subcoat wall comprised 70% hydroxypropyl cellulose having an average molecular weight of 60,000 and 30% poly(vinylpyrrolidone) identified as K29-32 having an average molecular weight of 40,000. The semipermeable wall comprised 99 wt % cellulose acetate comprising a 39.8% acetyl content and 1% polyethylene glycol comprising a 3,350 viscosity-average molecular weight. The dosage form comprised two passageways, 30 mils (0.762 mm), and it had a maximum risperidone release rate of 0.176 mg/hr.

1-46. (canceled)

47. An oral dosage form comprising:
a dose of a benzisoxazole derivative; and
an oral sustained release dosing structure adapted to sustainably release the benzisoxazole derivative in vitro such that:
(a) the release rate measured at 2 hours following administration of the oral dosage form does not exceed about 8 wt % of the dose per hour,
(b) the release rate measured at 6 hours following administration of the oral dosage form ranging from between about 6 to about 12 wt % of the dose per hour,
(c) the release rate measured at 10 hours following administration of the oral dosage form ranging from between about 12 to about 16 wt % of the dose per hour, or
(d) the release rate measured at 14 hours following administration of the oral dosage form does not exceed 8 wt % of the dose per hour,
wherein the release profile is determined using a USP Type VII bath indexer in a constant temperature water bath at 37° C.

48. The oral dosage form of claim 47, wherein the oral dosage form comprises an osmotic oral dosage form.

49. The oral dosage form of claim 47, wherein the benzisoxazole derivative comprises risperidone.

50. The oral dosage form of claim 47, wherein the benzisoxazole derivative comprises paliperidone.

51. A method comprising
(a) providing an oral dosage form comprising:
a dose of a benzisoxazole derivative; and
an oral sustained release dosing structure;
wherein the oral sustained release dosing structure sustainably releases the benzisoxazole derivative in vitro such that:
(b) the release rate measured at 2 hours following administration of the oral dosage form does not exceed about 8 wt % of the dose per hour,
(c) the release rate measured at 6 hours following administration of the oral dosage form ranging from between about 6 to about 12 wt % of the dose per hour,
(d) the release rate measured at 10 hours following administration of the oral dosage form ranging from between about 12 to about 16 wt % of the dose per hour, or
(e) the release rate measured at 14 hours following administration of the oral dosage form does not exceed 8 wt % of the dose per hour,
and wherein the release profile is determined using a USP Type VII bath indexer in a constant temperature water bath at 37° C.; and
(f) administering the oral dosage form to a patient.

52. The method of claim 51, wherein the oral dosage form comprises an osmotic oral dosage form.

53. The method of claim 51, wherein the benzisoxazole derivative comprises risperidone.

54. The method of claim 51, wherein the benzisoxazole derivative comprises paliperidone.

55. An oral dosage form comprising:
a dose of a benzisoxazole derivative; and
an oral sustained release dosing structure adapted to sustainably release the benzisoxazole derivative in vitro such that:
(a) the release rate measured at 2 hours following administration of the oral dosage form does not exceed about 4 wt % of the dose per hour,
(b) the release rate measured at 6 hours following administration of the oral dosage form ranging from between about 1 to about 4 wt % of the dose per hour,
(c) the release rate measured at 10 hours following administration of the oral dosage form ranging from between about 3 to about 7 wt % of the dose per hour, or
(d) the release rate measured at 24 hours following administration of the oral dosage form does not exceed 4 wt % of the dose per hour,
wherein the release profile is determined using a USP Type VII bath indexer in a constant temperature water bath at 37° C.

56. The oral dosage form of claim 55, wherein the oral dosage form comprises an osmotic oral dosage form.

57. The oral dosage form of claim 55, wherein the benzisoxazole derivative comprises risperidone.

58. The oral dosage form of claim 55, wherein the benzisoxazole derivative comprises paliperidone.

59. A method comprising:
(a) providing an oral dosage form comprising:
a dose of a benzisoxazole derivative; and
an oral sustained release dosing structure;
wherein the oral sustained release dosing structure sustainably releases the benzisoxazole derivative in vitro such that:
(b) the release rate measured at 2 hours following administration of the oral dosage form does not exceed about 4 wt % of the dose per hour,
(c) the release rate measured at 6 hours following administration of the oral dosage form ranging from between about 1 to about 4 wt % of the dose per hour,
(d) the release rate measured at 10 hours following administration of the oral dosage form ranging from between about 3 to about 7 wt % of the dose per hour, or
(e) the release rate measured at 24 hours following administration of the oral dosage form does not exceed 4 wt % of the dose per hour,
and wherein the release profile is determined using a USP Type VII bath indexer in a constant temperature water bath at 37° C.; and
(f) administering the oral dosage form to a patient.

60. The method of claim 59, wherein the oral dosage form comprises an osmotic oral dosage form.

61. The method of claim 59, wherein the benzisoxazole derivative comprises risperidone.

62. The method of claim 59, wherein the benzisoxazole derivative comprises paliperidone.

63. An oral dosage form comprising:
a dose D of a benzisoxazole derivative; and
an oral sustained release dosing structure adapted to sustainably release the benzisoxazole derivative at rates that provide (a) a mean, single dose, areas under a plasma concentration-time curve AUC of the benzisoxazole derivative and pharmacologically active metabolites thereof taken together for 0-3 hours AUC₀₋₃, 3-6 hours AUC₃₋₆, 6-9 hours AUC₆₋₉, 9-12 hours AUC₉₋₁₂, and 0-12 hours AUC₀₋₁₂ which satisfy the relationships:
AUC₀₋₃/AUC₀₋₁₂≧0.18,
AUC₃₋₆/AUC₀₋₁₂≧0.18,
AUC₆₋₉/AUC₀₋₁₂≧0.18,
and
AUC₉₋₁₂/AUC₀₋₁₂≧0.18.

64. The oral dosage form of claim 63, wherein the oral dosage form comprises an osmotic oral dosage form.

65. The oral dosage form of claim 63, wherein the benzisoxazole derivative comprises risperidone.

66. The oral dosage form of claim 63, wherein the benzisoxazole derivative comprises paliperidone.

67-142. (canceled)
